(19) United States
(12) Patent Application Publication
(10) Pub. No.: US 2016/0051533 A1
(43) Pub. Date: Feb. 25, 2016

(54) DIAGNOSIS AND PHARMACOLOGICAL TREATMENT OF SEASONAL AFFECTIVE DISORDER AND SYMPTOMS OF SEASONALITY

(71) Applicant: Ladd Research LLC, Bethesda, MD (US)

(72) Inventor: Thatcher B. Ladd, Bethesda, MD (US)

(21) Appl. No.: 14/464,172

(22) Filed: Aug. 20, 2014

(51) Int. Cl. A61K 31/472; A61K 31/4184

(52) U.S. Cl. CPC A61K 31/472; A61K 31/4184

(57) ABSTRACT

A method for reducing one or more symptoms associated with seasonal affective disorder and/or seasonality in symptoms of bipolar and/or premenstrual syndrome in an individual, the method including administering to the individual suffering from seasonal affective disorder and/or more generally, seasonality symptoms, a composition that comprises a therapeutically effective amount of an agent to down regulate the activity of an orexin selected from the group consisting of orexin A or orexin B, at one or more orexin receptors.

### BACKGROUND AND BRIEF SUMMARY OF THE INVENTION

The present invention is directed to the fields of neuroscience and in particular, to pharmacological methods for treating Seasonal Affective Disorder (SAD) and other medical disorders that are caused by the physiological mechanism newly found to cause seasonality, including SAD.

As described previously by Smart and Jerman 2002 (Pharmacology & Therapeutics 94, pgs 51-61), orexin A and orexin B, less frequently referred to as hypocretin A and B, are neuropeptides with amino acid residue lengths of 33 and 28, respectively. Both orexins are derived from prepro-orexin, a larger neuropeptide with a length of 130 amino acids. Orexin A has two disulfide bonds and orexin B has none. Orexins are produced by a small number of neurons located mostly in the hypothalamus, but these neurons send axonic projections throughout the brain. These neuropeptides generally act on post-synaptic G-protein coupled receptors named orexin receptor 1 and orexin receptor 2 (OxR1, OxR2). While orexin-A binds to both receptors 1 and 2 with equal affinity, orexin B has a greater affinity for receptor 2 than for receptor 1. Orexins are strongly conserved neuropeptides across vertebrate species.

Generally thought of as an excitatory neuropeptide, orexin-bound orexin receptors usually cause an intracellular transient Ca2+ spike via intracellular G-protein mediated signaling. The OxR1, as documented in Tsujino and Sakurai 2009, a pharmacological review for The American Society for Pharmacology and Experimental Therapeutics (Vol. 61, No. 2), is coupled with G-Q/11 class of G-proteins and is thought to act via intracellular phosphatidylinositol signaling cascade. It has been found that OxR2 may be coupled with Gq, Gs, and Gi proteins, offering an array of possible downstream effects.

Orexin producing neurons have been found only in the perifornical area, the lateral hypothalamic area, and the posterior hypothalamic area in rats and humans, though projections have been found throughout the brain. Differential distribution in the central and peripheral nervous systems have been found between the two types of orexin receptors. OxR1 s have been found specifically in the adrenal glands and kidneys, the thyroid, testis, ovaries, and jejunum, while OxR2s have been found in the adrenal and thyroid glands, and the lungs. OxR1 s and OxR2s have been found in many regions of the central nervous system. By targeting orexin neuronal firing rates, the prepro-orexin abundance, orexin A and B stability, or antagonizing the OxR1 and OxR2 and any other orexin-related mechanisms, it is possible to decrease the mood and physiological symptoms of seasonality disorders. By decreasing orexin activity, the severity of seasonally presenting symptoms can be therapeutically reverted back to diagnostically insignificant levels present in the spring and summer.

Known colloquially as Seasonal Affective Disorder, or SAD, seasonal symptoms in mood, activity, and appetite have been documented in humans for millennia. Hippocrates describes seasonally caused changes in mood circa 400 BC. SAD is in fact a misnomer, as human seasonality presents with physiological and metabolic symptoms, not just mood changes. But, because human seasonality is commonly referred to as SAD, the disclosure and exemplary research set forth below employ SAD to refer to human seasonality as well as the specific disorder of SAD. SAD is one of the few psychiatric disorders with relative high prevalence rates that has a clear environmental cause. Symptoms of SAD are present in the fall and winter, as the days are getting shorter and light intensity across the spectrum is decreasing, including infrared and UV radiations. These symptoms usually remit completely in the spring and summer, when days are getting longer and light intensity is increasing. Symptoms of SAD include, but are not limited to: anxiety, depression, lethargy, hypersomnia, tiredness, aches, carbohydrate and other food cravings, weight gain, and atypical symptoms of depression.

Patients diagnosed with SAD experience significant social and functional impairment during the fall and winter. In one study, Pendse et al. 2003 (European Psychiatry, pgs. 36-39), found no statistically significant differences in the Montgomery-Asberg Depression Rating Scale (MADRS) and the Interview Schedule for Social Interaction (ISSI) scores, between current SAD patients and patients who had attempted suicide (SA). Both SAD and SA patients had significantly worse scores than the healthy controls. The study demonstrates that SAD causes poor scores on many of the parameters of the MADRS and ISSI tests, including adequacy of social integration, availability of attachment, and adequacy of attachment. Similarly, Eagles et al. 2002 (British Journal of Psychiatry, 180, pgs 449-454) found that SAD patients had higher annual rates of primary care consultations, purchased more prescriptions, performed more medical tests, and had more specialist referrals than matched controls. SAD patients were found to have higher rates of psychiatric and urinary symptoms, tiredness, weight gain, depression, heart palpitations, and premenstrual syndrome symptoms than controls.

It has been found that average antidepressant medication prescriptions have seasonal fluctuations. This is most likely due to attempts to self medicate SAD and sub-syndromal SAD symptoms with medications that do not target the underlying biological cause. Similarly, SAD patients often purchase SAD light therapy lamps for self-treatment despite the poor efficiency of light therapy, its cost, lack of portability, and the necessity of daily use. What is needed for the proper treatment of SAD, and what is outlined here, is both the identification of the cause of SAD, identified here as an increase in orexin activity as light decreases in the fall and winter, and its novel treatment via chronic dosing during these periods of reduced light, with dual or single orexin receptor antagonists, or other pharmacological interventions which suppress orexin activity.

Previous studies have not shown the effectiveness of pharmacological intervention to down regulate the orexin system as a treatment in an animal model of SAD. Additionally, there has recently been a push in SAD animal modeling to move from nocturnal lab rat strains, like Wistar and Sprague-Dawley, to new diurnal small mammal species, like fat sand rats and Mongolian gerbils, as humans are diurnal not nocturnal. The reasons behind this push are unclear. Because diurnal mammal's neuronal photoperiod tracking mechanisms, like those in the suprachiasmatic nucleus, evolved during the day in direct sunlight, they may be hard or impossible to shift with relatively weak artificial lab light. Nocturnal species, like the Wistar rats used in the research serving as a basis for the present invention, on the other hand, evolved photoperiod tracking mechanisms that could be reset with only the weak light of dusk and dawn, which may be recreated in a lab setting. Previous studies on nocturnal lab rats with static photoperiods have already shown that shorter light schedules tend to increase depressive and anxiety behaviors (Prendergast and Kay 2008, Journal of Endocrinology, 20, pgs 261-267). Those studies, however, have not shown orexin receptor antagonism as an effective treatment for symptoms of their SAD models.

Depression and Seasonal Affective Disorder are caused by different biological mechanisms and have different symptoms and pathologies. Depression is one of many symptoms of increased seasonality; it is not the disorder itself. Depression and SAD have different causative mechanisms. SAD is known to be caused by light differences, including changes in light intensity, infrared radiation, ultraviolet (UV) radiation, and photoperiod, in the fall and winter versus the spring and summer. On the other hand, depression's cause is unknown and could involve many different psychosocial and biological mechanisms. More importantly, Major Depression (MD) and the depressive symptoms of SAD may actually present differently, though they have been named similarly. Furthermore, it is possible for SAD patients to present with a symptom profile that both does not contain depression as a symptom and is significant enough to warrant drug therapy. Depression is not SAD, and SAD does not necessarily contain depressive symptoms.

Arendt et al. 2013 (Arendt et al. 2013 “Depressive Behavior and Activation of the Orexin/Hypocretin System”. Behavioral Neuroscience. 127: pgs 86-94), correlated orexin immunohistochemistry by brain region in mice with performance in a Forced Swim Test (FST). Mice were on normal light schedules, which did not attempt to model SAD. Arendt et al. found that, in some brain regions, decreased orexin activity correlated with less depressive-like scores in the FST. In other brain regions, increased or decreased orexin activity correlated with depressive-like scores in the FST. Ultimately, this part of the study was correlational, and was not related to seasonality. No drug or photoperiod manipulations or were used in Arendt et al. Furthermore, drugs do not generally target brain regions differentially.

In humans, no cerebrospinal fluid (CSF) or other biological sampling of orexin activity has been done in SAD patients compared to healthy controls. In humans, orexin CSF levels have not been found to correlate with depression. Salomon et al. 2003 (Salomon et al. “Diurnal Variation of Cerebrospinal Fluid Hypocretin-1 (Orexin-A) Levels in Control and Depressed Subjects” Biological Psychiatry 54. pgs 96-104) found no statistical significance in CSF orexin mean 24-hour concentration, daytime concentration, or nighttime concentration. They found slight day/night amplitude differences between depressed and control patients, and hypothesized that this was due to “dampened” or decreased diurnal orexin variation in depressed patients. Similarly, Schmidt et al. (Schmidt et al. 2011. “CSF-hypocretin-1 levels in patients with major depressive disorder compared to healthy controls”. Psychiatry Research. 190. pgs 240-243) found no statistical significance in CSF orexin levels between depressive patients and healthy controls.

To reiterate, standard depression-like characteristic testing does not model SAD in rodents. To model SAD in rodents, the behavioral tests must be paired with a photoperiod manipulation that attempts to simulate seasons. Similarly, because SAD can present without depression as a symptom, the best way to test pharmacological therapies for SAD in a rodent model is by testing the drug on fall/winter-like photoperiod induced differences in multiple behavioral tests. Accordingly, in the exemplary research (see the below Examples) dual orexin receptor antagonists were tested in their ability to treat short-photoperiod induced symptoms in the Open Field Test (OFT), Forced Swim Test (FST), Elevated Plus Maze (EPM), Marble Burying Test (MBT), and in Growth Rate (GR).

Some previous research without photoperiod manipulation has shown anti-depressant potential of orexin receptor antagonists. In mice, Scott et al. (Scott et al. 2011. “Hcrtr1 and 2 signaling differentially regulates depression-like behaviors”. Behavioural Brain Research. 222. pgs 289-294) found that OxR1 null and SB-334867, an OxR1 antagonist, display decreased immobility in the Forced Swim Test and Tail Suspension Test. Nollet et al. 2012 (Nollet et al. “Neurogenesis-Independent Antidepressant-Like Effects on Behavior and Stress Axis Response of a Dual Orexin Receptor Antagonist in a Rodent Model of Depression”. Neuropsychopharmacology. 37. pgs 2210-2221) tested a dual orexin receptor antagonist in an Unpredictable Chronic Mild Stress (UCMS) paradigm. UCMS paradigms induce changes in anxiety and depression tests by subjecting mice to random daily stressors, like overnight illumination, change of cage mate, food and water deprivation, and cage tilt. Nollet et al. found, in this paradigm, that mice treated with a dual orexin receptor antagonist displayed less depression-like characteristics in the Tail Suspension Test, less anxiety-like characteristics in the Elevated Plus Maze, and changed parameters in the Resident-Intruder Test, and the Novelty-Suppressed Feeding Test. UCMS does not and has not been used as a valid model of SAD.

The research serving as a basis for the present invention used light/dark (L/D) photoperiod shifts to induce SAD (fall/winter) and non-SAD (spring/summer) behavioral profiles in rats, and treated the behavioral profile of SAD with two dual orexin receptor antagonists. It was proven that drugs down regulating the orexin system have statistically significant effects in treating the disparity of symptoms between long light (LL), or non-SAD, and short light (SL), SAD, by treating the SL-induced symptom profile. The research serving as a basis for the present invention identifies the newly found mechanism that induces short photoperiod behavioral symptoms in multiple behavioral tests as increases in orexin, and treats this mechanism by the pharmacological down-regulation of the orexin system.

To expedite the effective pharmacological treatment of SAD in humans, what was needed was a way to more reliably induce SAD symptoms in nocturnal lab rats, and a confirmed hypothesis about the biological cause of SAD. Here, the causative biological cue in SAD in all mammals that both live at seasonal latitudes and stay active during the winter is identified as an increase in orexin activity. The increase in orexin activity occurs during the fall and winter, and may come about through multiple mechanisms. For example, increased orexin activity may be caused by: increases in OxR1 and OxR2 densities in the postsynapses of neurons receiving orexin neuronal efferent inputs, decreased degradation of orexin A and orexin B, increases in prepro-orexin production, increased activity of orexin neurons via multiple mechanisms, and many other mechanisms. Therapeutically, antagonizing the orexin receptors can block these multiple mechanisms and, thus, decrease orexin activity.

To investigate and determine the cause of many symptoms of SAD in order to develop an effective and efficacious treatment of SAD and other seasonally affected disorders, research was conducted which resulted in demonstrating a newly found mechanism by which short photoperiods induce behavioral symptoms in multiple behavioral tests, as well as the effectiveness of a therapy which pharmacologically down regulates the orexin system.

The present invention is contrary to some previous findings in the art (Deats, S. P. et al., “Attenuated orexinergic signaling underlies depression-like responses induced by daytime light deficiency” Neuroscience 272 (2014) pgs 252-260), wherein the treatment methods of the present invention stand in recognition that it takes many weeks of decreasing photoperiods and light intensity for humans to exhibit SAD symptoms. Contrary to the research serving as a basis for the present invention, Deats et al. hypothesized that decreases in orexin activity may underlie SAD. This conclusion is predicated on the behavioral effects of only one dose of a drug targeting the orexin system. In contrast, chronic effects of increases or decreases in orexin activity may differ from acute changes in orexin activity. The research underlying the present invention found that SAD symptoms may present as the result of a month or more of chronic increases in orexin activity. The research, accordingly, administered orexin receptor antagonists chronically for 4 weeks, and found a robust therapeutic effect in the treatment of SAD-induced symptoms in rats. The research, explained in subsequent sections, shows the therapeutic effects of administering orexin receptor antagonists to reduce SAD-induced symptoms.

The results of the present research led to novel and unexpected methods to act on the physiological mechanism underlying SAD. By administering orexin receptor antagonists, the symptoms of SAD and similar seasonal disorders can be successfully reduced.

The present invention thus provides several embodiments including those which encompass a method for reducing one or more symptoms associated with seasonal affective disorder in an individual. The afflicted individual includes any warm-blooded vertebrates including humans and domesticated animals that encompass, for example, but not limited to livestock, horses, cats, dogs and other companion pets.

One embodiment is directed to a method comprising administering to an individual suffering from seasonal affective disorder, a composition comprising a therapeutically effective amount of an agent to down regulate the activity of an orexin selected from the group consisting of orexin A or orexin B, at one or more orexin receptors. The symptoms associated with SAD include any one or all of depression, anxiety, lethargy, hypersomnia, tiredness, aches, carbohydrate and other food cravings, weight gain and atypical symptoms of depression, or other symptoms.

Down regulating the activity of orexin via orexin A or B probably entails a type of inactivation. The down regulating orexin activity at orexin receptors probably entails antagonism or decreasing receptor numbers.

The agent comprises an orexin receptor antagonist selected from the group consisting of an orexin receptor antagonist selective for orexin 1 receptors, an orexin receptor antagonist selective for orexin 2 receptors, and an orexin receptor antagonist having affinity at orexin 1 receptors and orexin 2 receptors. More specifically, the agent may be selected from but is not limited to the group consisting of almorexant, TCS 1102 and proline bis-amide orexin antagonists.

Another embodiment of the present invention is directed to a method for reducing one or more seasonal symptoms associated with bipolar disorder in an individual, the method comprising administering to the individual suffering from seasonal symptoms associated with bipolar disorder, a composition comprising a therapeutically effective amount of an agent to down regulate the activity of an orexin selected from the group consisting of orexin A or orexin B, at one or more orexin receptors.

A further embodiment of the present invention is directed to a method for reducing one or more seasonal symptoms associated with premenstrual syndrome in an individual, the method comprising administering to the individual suffering from seasonal symptoms associated with premenstrual syndrome, a composition comprising a therapeutically effective amount of an agent to down regulate the activity of an orexin selected from the group consisting of orexin A or orexin B, at one or more orexin receptors.

Another embodiment of the present invention is a method for reducing one or more symptoms associated with seasonality in an individual, the method comprising administering to the individual suffering from a seasonality disorder, a composition comprising a therapeutically effective amount of an agent to down regulate the activity of an orexin selected from the group consisting of orexin A or orexin B, at one or more orexin receptors.

Another embodiment of the present invention is a bioanalysis for SAD treatment comprising a method of determining whether an individual is responsive to a treatment for seasonal affective disorder, the method including determining the levels of orexin in a biological sample from the individual obtained once in the spring or summer and once during the fall or winter prior to said treatment, and determining a difference in level of orexin; administering said treatment for period of time to down regulate orexin receptor activity; determining the levels of orexin in a biological sample from the individual during or after said treatment period, comparing the levels of orexin present in the sample obtained after initiating treatment to a baseline level of orexin present in the sample obtained prior to treatment during spring or summer, and determining that the individual is responsive to treatment if orexin levels are reduced during or after said treatment period.

Additional aspects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and do not restrict the invention, as claimed.

### BRIEF DESCRIPTION OF THE DRAWINGS

FIG. 1 is a graph showing OFT distance travelled as % Distance in Center in C-DMSO, LL-DMSO, SL-DMSO and SL-TCS groups.

FIG. 2 is an OFT graph showing distance travelled in cm (A) in C-Wat, LL-Wat, SL-Wat and SL-Almo groups.

FIG. 3 is an OFT graph showing monitored thigmotactic behavior in animals treated with IP TCS 1102.

FIG. 4 is an OFT graph showing decreases in normally positively rewarding exploratory behaviors induced by photoperiod changes that can be treated with TCS.

FIG. 5 is an OFT graph showing that even if changes in rat distance traveled on an individual scale are not addressed, the rescue of the withdrawal-related behavior with a p value of 0.06 is still significant.

FIG. 6 is an OFT graph showing that the rats that were under the short photoperiod spent less time, as well as less distance in the hyper center of the OFT.

FIG. 7 is an EPM graph showing the percentage of time spent in open arms (% TOA).

FIG. 8 is an EPM graph showing the percentage of open arm entries (% OAE).

FIG. 9 is an EPM graph showing the number of arm entries (AE).

FIG. 10 is an EPM graph showing an increase and treatment of anxiety-like behaviors in the EPM.

FIG. 11 is a FST graph showing significant differences vs. C-Wat and SL-Wat groups with a rescue of the SL condition with drug treatment.

FIG. 12 is a FST graph showing significant difference vs. C-Wat group and close-to-significant difference vs. SL-Wat group.

FIG. 13 is a FST graph showing FST 2 binned results for climbing time for both SL-Veh mean and SL-Almo mean in a double blind reanalysis of FST video recording.

FIG. 14 is a FST graph showing FST 2 binned results for floating time for both SL-Veh mean and SL-Almo mean.

FIG. 15 is a FST graph showing FST 2 binned results for climbing time for both CL (control light)-Veh mean and LL-Veh mean.

FIG. 16 is a FST graph showing FST 2 binned results for floating time for CL-Veh mean, LL-Veh mean, SL-Veh mean and SL-Almo mean.

FIG. 17 is a FST graph showing FST 2 binned results for climbing time for both SL-Veh mean and LL-Veh mean.

FIG. 18 is a FST graph showing FST 2 binned results for floating time for both LL-Veh mean and SL-Veh mean.

FIG. 19 is a FST graph showing FST 2 binned results for climbing time for CL-Veh mean, LL-Veh mean, SL-Veh mean and SL-Almo mean.

FIG. 20 is a MBT graph differentiating LL, SL, and SL-treated groups for marbles greater than ⅓ buried.

FIG. 21 is a MBT graph showing treatment of the SL condition with drug therapy for marbles less than ⅓ buried.

FIG. 22 is a MBT graph showing treatment of the SL condition for marbles ⅓ buried.

FIG. 23 is a MBT graph showing that almorexant reduced the increase of unburied marbles induced by SL.

FIG. 24 is a MBT graph showing that almorexant reduced the decrease of ⅔ buried marbles induced by SL.

FIG. 25 is a graph showing growth rate during TCS 1102 treatment in the IP groups.

FIG. 26 is a graph showing growth rate during TCS 1102 treatment for the SL-Veh vs LL-Veh groups.

FIG. 27 is a graph showing growth rate during TCS 1102 treatment demonstrating the effect of IP DORA.

FIG. 28 is a graph showing growth rated during OG almorexant treatment.

FIG. 29 is a graph showing growth rate during almorexant treatment for LL-Veh vs SL-Veh.

FIG. 30 is a graph showing growth rate during OG almorexant treatment for SL-Veh vs SL-Almo.

### DETAILED DESCRIPTION OF THE INVENTION

The present invention thus provides several embodiments which encompass pharmacological methods for treating Seasonal Affective Disorder (SAD) and other medical disorders having seasonality symptoms that are caused by the physiological mechanism newly found to cause SAD. The present methods reduce one or more symptoms associated seasonality and more specifically, with seasonal affective disorder in an individual. The afflicted individual includes any warm-blooded vertebrates including humans and domesticated animals that encompass, for example, but not limited to livestock, horses, cats, dogs and other companion pets.

One embodiment is directed to a method comprising administering to an individual suffering from seasonal affective disorder, a composition comprising a therapeutically effective amount of an agent to down regulate the activity of an orexin selected from the group consisting of orexin A or orexin B, at one or more orexin receptors. The symptoms associated with seasonal affective disorder are not limited to any one or all of depression, anxiety, lethargy, hypersomnia, tiredness, aches, carbohydrate and other food cravings, weight gain and atypical symptoms of depression, or other symptoms. In several alternatives: orexin receptor activity is down regulated at an orexin 1 receptor; orexin receptor activity is down regulated at an orexin 2 receptor; and orexin receptor activity is down regulated at both orexin 1 and orexin 2 receptors. The method includes the option wherein the expression of an orexin receptor is decreased. In a couple of alternatives: orexin receptor activity is down regulated through inactivation of orexin A; and orexin receptor activity is down regulated through inactivation of orexin B.

The active agent includes an orexin receptor antagonist selected from the group consisting of an orexin receptor antagonist selective for orexin 1 receptors, an orexin receptor antagonist selective for orexin 2 receptors, and an orexin receptor antagonist having affinity at orexin 1 receptors and orexin 2 receptors. More specifically, the agent is not limited to any one or all of almorexant, TCS 1102 and proline bis-amide orexin antagonists.

Another embodiment of the present invention is a method for reducing one or more symptoms associated with seasonality in an individual, the method comprising administering to the individual suffering from a seasonality disorder, a composition comprising a therapeutically effective amount of an agent to down regulate the activity of an orexin selected from the group consisting of orexin A or orexin B, at one or more orexin receptors.

Research was performed, as explained in the below Examples, demonstrating that an increase in orexin activity causes the many symptoms of SAD in both lab rats and by extension to humans. This was tested in a novel dynamic light-shifting paradigm, and with the new use of an existing class of compounds, orexin receptor antagonists. Used in this research, Almorexant HCL and TCS1102, both dual orexin receptor antagonists (DORA), effectively and statistically significantly treated the photoperiod induced SAD symptoms in Wistar rats.

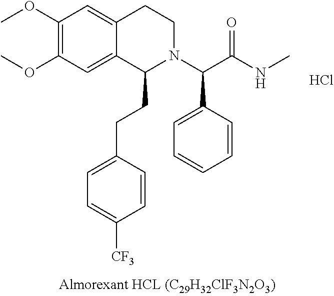

Separated by high performance liquid chromatography and verified by mass spectrometry and HNMR spectroscopy, the purity of the Almorexant HCL (Almo) used in this study was ≧99%. The other dual orexin receptor used in this study was TCS1102.

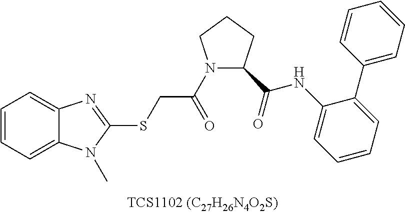 [SMILES: Cn1c(SCC(=O)N2CCC[C@H]2C(=O)Nc2ccccc2-c2ccccc2)nc2ccccc21]

Similarly, separated by high performance liquid chromatography and verified by mass spectrometry and HNMR spectroscopy, the purity of the TCS1102 was ≧99%. The high levels of purity for these two research grade specific orexin receptor antagonists allowed for the precise targeting of the orexin system.

These drugs treated a robust SAD symptom profile in the rats induced by a novel dynamic photoperiod shift. Previous studies, like previously cited Prendergast and Kay 2008, use static photoperiod shift of for example 10 hours light/14 hours dark (10 L/12 D) for their short light (SL) group, 12 L/12 D for their control light (CL) group, and 14 L/10 L for their long light (LL) group. In nature, static and unshifting photoperiods like this do not exist at the latitudes where SAD is present. To remedy this, for this research, all light groups started at 12 L/12 D; the LL group, which aimed to replicate a spring and summer behavioral profile, increased light duration by 8 minutes/day for 8 weeks. The SL group, which aimed to replicate the behavioral profile of SAD in fall and winter months, decreased the light duration by 8 minutes/day for 8 weeks. The CL group had a L/D schedule constant at 12 L/12 D. For the LL group, four minutes were added to either end of the light period each day. In other words, the LL group had lights on 4 minutes earlier and off 4 minutes later every day during the experiment. Another alteration to the norm of rat SAD behavioral modeling is that all non-ovarectomized female Wistar rats were used. Female humans with SAD are both more prevalent than males with SAD, and tend to have worse symptoms than males.

Using the L/D photoperiod shift to induce SAD and non-SAD behavioral profile in rats along with two dual orexin receptor antagonists, it was proven that drugs down regulating the orexin system have statistically significant effects in treating the disparity of symptoms between LL, or non-SAD, and SL, or SAD, groups. The results of the present research led to novel and unexpected methods to act on the physiological mechanism underlying SAD. By administering orexin receptor antagonists chronically, the symptoms of SAD and similar seasonal disorders can be successfully reduced.

More specifically, the research was to: 1) to investigate the effects of progressive changes—shortening and lengthening—of the daylight duration on anxiety-related, depression-related, and other SAD-related behavior in rats, and 2) to investigate the effects of two dual orexin receptor antagonists, almorexant and TCS 1102, on potential anxiety-like, depressive-like, and other SAD-like states induced by progressive shortening of the daylight duration.

The methods employed in the research included the use of Wistar female rats that were housed for an 8-week period in three different conditions: a constant light/dark (L/D) 12 h/12 h cycle (Control condition: C or CL), a progressive lengthening of the daylight duration (Long Light condition (LL): +8 min per day, +4 min at the beginning of the cycle and +4 min at the end of the cycle (light duration: started at 12 h and ended at 19 h 30 min from the beginning to the end of the experiment) and a progressive shortening of the daylight duration (Short Light condition (SL): −8 min per day, −4 min at the beginning of the cycle and −4 min at the end of the cycle (light duration: started at 12 h and ended at 4 h 30 min from the beginning to the end of the experiment).

At the beginning of the 5^{th} week of L/D change, the group housed in SL condition was divided into three groups which received one administration per day of TCS 1102 (20 mg/kg, intraperitoneal: IP), almorexant (100 mg/kg, per-os (PO) i.e. oral administration), or their vehicles (20% DMSO (dimethylsulfoxide) IP for half of animals or water PO for the other half). Animals of CL and LL groups received the vehicles in the same conditions.

Animals were subjected to three tests for examination of anxiety related SAD behavior, the open-field test (two sessions at 1 week and 2.5 weeks of drug treatment, respectively), the elevated-plus maze test (one session at 2 weeks of treatment) and the marble burying test (one session at 3 weeks of treatment), and one test for examination of depression-related behavior, the forced swimming test (FST; two sessions at 1.5 and 3.5 weeks of treatment, respectively).

Rats were weighed three times a week in order to examine the effect of L/D changes and the effects of treatments on the body weight.

The results demonstrated that:

1) The IP injection of DMSO induced anxiogenic-like effects and depressant-like effects which depended on the L/D condition.

2) The LL condition induced an anxiolytic-like effect in the EPM test, but not in the open-field test, increased the activity in these two tests and induced an antidepressant-like effect in the FST. These effects were observed in rats treated with water but not in those injected with DMSO. The LL condition also increased the body weight gain.

3) Conversely, the SL condition induced an anxiogenic-like effect in the EPM test and in the open-field test (only at the first session) in rats injected with DMSO, but not in those administered with water. The SL condition did not modify the depression-related behavior in the FST. The SL decreased the number and depth of marbles buried in the MBT compared to the LL condition. The SL condition also decreased the body weight gain.

4) TCS 1102 (20 mg/kg IP) reduced the anxiogenic-like effect of SL condition in the open-field test and tended to reduce the anxiogenic-like effect of SL condition in the EPM test. TCS 1102 had no significant effect on depression-related behavior in the FST and on the body weight.

5) Almorexant (100 mg/kg) did not significantly modify the anxiety-related behavior in the EPM test and in the open-field test, increased the activity in the EPM test, induced an antidepressant-like effect in the FST, treated the decrease in marbles buried and marble depth in the MBT compared to the LL, and reduced the decrease of body weight gain induced by SL condition. These effects of almorexant were similar as those induced by LL condition.

The present study showed that:

a) A progressive lengthening of the daylight duration induced an anxiolytic effect and an antidepressant effect.

b) A progressive shortening of the daylight duration induced an anxiogenic-like effect, and a SAD behavioral profile consistently different from LL.

These effects were dependent on other manipulations which may induce slight stress, such as the administration of vehicles. Therefore, because TCS 1102 and almorexant were administered by different routes and in different vehicles, it was difficult to compare their effects. The results show that:

a) TCS 1102 reduced the anxiogenic-like and SAD-like effect of a shortening of daylight duration.

b) Almorexant induced an antidepressant effect and treated SAD symptoms in animals subjected to a shortening of daylight duration, i.e. induced the same effect as a lengthening of the daylight duration.

A progressive lengthening of the daylight duration induced an anxiolytic effect and an antidepressant effect. These effects are consistent with past studies which showed that long photoperiod produce antidepressant-like effect in nocturnal rats. Conversely, a progressive shortening of the daylight duration induced an anxiogenic-like effect.

The research showed that TCS 1102 reduced the anxiogenic-like effect of a shortening of daylight duration, and that almorexant induced an antidepressant effect in animals subjected to a shortening of daylight duration, i.e. induced the same effect as a lengthening of the daylight duration.

The research showed that dual orexin receptor antagonists counteract SL-induced changes in 4 different tests and physiological measures: the OFT1, EPM, MBT, and BW. Furthermore, in the FST2, it showed that LL or a “summer” photoperiod regimen causes an antidepressant effect that is similar to that of a dual orexin receptor antagonist.

Wistar rats, and strains derived from Wistar rats, are the most commonly employed animal model for testing drug therapies for human neuropsychiatric conditions. Wistar models of anxiety, depression, and SAD have previously been shown to have face, construct, and most importantly, predictive validity in the testing of potential drug therapies for humans. Molina-Hernandez and Tellez-Alcantara 2000 (Molina-Hernandez and Tellez-Alcantara. “Long Photoperiod Regimen may Produce Antidepressant Actions in the Male Rat”. Progress in Neuro-Psychopharmacology & Biological Psychiatry. Vol. 24. Issue 1. pgs 105-116) found that their Wistar SAD model predicted the therapeutic effect of the tri-cyclic antidepressants, clomipramine and desipramine-HCl, known drugs for the treatment of SAD and depression in humans. Hirose et al. (Hirose et al. 2004. “Mechanism of action of aripiprazole predicts clinical efficacy and a favourable side effect profile”. Journal of Psychopharmacology. 18: pgs 375-383) found that a Wistar model of atypical depression predicted the therapeutic effect of aripiprazole, an atypical antidepressant, in humans. Leveleki et al. (Leveleki et al. 2006. “Pharmacological evaluation of the stress-induced social avoidance model of anxiety”. Brain Research Bulletin. 69: pgs 153-160) found that their Wistar model of anxiety via social avoidance predicted the anxiolytic effects in humans of two benzodiazepines, chlorodiazepoxide and diazepam, a selective serotonin reuptake inhibitor, fluoxetine, and the serotonin partial agonist buspirone, Bennett et al. (Bennett et al. 1982. “Comparison of the actions of trimethadione and chlordiazepoxide in animal models of anxiety and benzodiazepine receptor binding”. Neuropharmacology. 21: pgs 1175-1179) found that a Wistar model of anxiety predicted the anxiolytic effect of the benzodiazepine chlordiazepoxide in humans. Hogg et al. (Hogg et al. 2006. “Prediction of anti-panic properties of escitalopram in the dorsal periaqueductal grey model of panic anxiety”. Neuropharmacology. 51: pgs 141-145) found that their Wistar model of panic anxiety predicted the anxiolytic effect of the SSRI escitalopram. Malatynska et al. (Malatynska et al. 2002. “Reduction of Submissive Behavior in Rats: A Test for Antidepressant Drug Activity”. Pharmacology. 64: pgs 8-17) found that their Wistar model of depression predicted the anti-depressant effects of fluoxetine, an SSRI, and desipramine, a tri-cyclic antidepressant. Ultimately, the above are not by any means an exhaustive list of the efficacy of Wistar rat modeling's predictive validity. They serve to demonstrate that Wistar rat behavioral models are a valid method for predicting the therapeutic effect of drugs in humans.

The two drugs used in the exemplary research, TCS1102 and Almorexant HCl, are research-grade dual orexin receptor antagonists that indicate a powerful therapeutic effect of their class of medication, orexin receptor antagonists, on SAD symptoms. Because these drugs were tested in a SAD model on Wistar rats, the findings of efficacy are likely translational to humans. These orexin receptor antagonists (ORAs) also indicate a therapeutically effective method of treating SAD by chronically, not acutely, down-regulating the orexin system by receptor antagonism.

The exemplary research for the present invention (see below Examples) utilized two dual orexin receptor antagonists with differing pharmacokinetics and molecular structures to demonstrate that the therapeutic effect of these drugs came from their action at the OxRs, not from other effects. Furthermore, it is standard to extrapolate to the therapeutic potential of a class of medications after testing two in a rat model. Johnson et al. 2010. (Johnson et al. 2010. “A key role for orexin in panic anxiety”. Nature Medicine. 16: 111-115) indicated the therapeutic potential of all OxR1 antagonists for the treatment of panic disorder after testing two: SB334867 and SB408124. Specifically, Johnson et al. “suggest that ORX-1 receptor antagonists may provide a new therapeutic approach for the treatment of Panic Disorder”. The drugs used were research-grade and chemically analyzed for ≧99% purity. The doses used in the study were 100 mg/kg/day of Almorexant HCl delivered with oral gavage (OG or PO) with distilled water as a vehicle and 20 mg/kg/day of TCS 1102 delivered with intra-peritoneal injection (IP) with 20% Dimethyl sulfoxide (DMSO)/80% distilled water as a vehicle. Almorexant and TCS 1102 were supplied by Green Pharma, France, with batch numbers P3187928 and P7522747, respectively.

Malherbe et al. (Malherbe et al. 2009. “Biochemical and Electrophysiological Characterization of Almorexant, a Dual Orexin 1 Receptor (OX1)/Orexin 2 Receptor (OX2) Antagonist: Comparison with Selective OX1 and OX2 Antagonists”. Molecular Pharmacology. 76:618-631) found that [3H] Almorexant bound to one saturable site on recombinant human OxR1 with a Kd of 1.3 nM, and to human OxR2 with a Kd of 0.17 nM. Almorexant was classified as a competitive antagonist at hOxR1 and non-competitive antagonist at hOxR2. Almorexant had a fast binding and dissociation rates at hOxR1 but not hOxR2. Almorexant, when acting on rat ventral tegmental area dopamine neurons, was shown by Malherbe et al. to counteract the excitatory effects of orexin-A. 0.0001-10 micro molar orexin A was shown to alter basal firing frequency to 175+/−17% of control in nearly half of the tested neurons. 1 micro molar of Almorexant was shown to completely block the effect of orexin A. A finding demonstrated by the exemplary research is that fall/winter increases in orexin activity may act via increased orexin A release onto post-synaptic targets. Almorexant, a drug shown to counteract the activity of orexin A, treated symptoms of SAD in the exemplary Wistar model, thus supporting the theory and indicating the effectiveness of DORAs for the treatment of SAD.

TCS 1102, a proline-bis amide orexin antagonist, has been described by a distributor, Tocris BioSciences, Cat. No. 3818, as having Ki values of 3 nano molar for OxR1 and 0.2 nano molar for OxR2. Less research has been done on TCS 1102 than has been done on Almorexant, as the latter was initially developed as a potential sleep aid in humans.

These two drugs used in the study have different pharmacokinetic profiles, bioavailabilities, dissociation rates, and mechanisms of antagonism. Furthermore, they were delivered via different administration routes, OG or IP, and were dissolved in different vehicles at different doses. What was held constant was their therapeutic target: antagonism of the orexin receptors. The effective doses given in the study may not scale directly for human use. Reagan-Shaw et al. (Reagan-Shaw et al. 2007. “Dose translation from animal to human studies revisited”. The FASEB Journal. 22: pgs 659-661) noted that animal doses in pharmacological studies should not be extrapolated to human doses with simple conversion. A more effective method, they propose, is via body-surface-area body surface area (BSA) calculations. But, though BSA extrapolation is more effective than direct proportionality, it is by no means perfect. Reagan-Shaw et al. also noted that BSA calculations do not take into account drug elimination, among other factors, which may confound dose scaling to humans. Sharma and McNeill (Sharma and McNeill 2009. “To scale or not to scale: the principles of dose extrapolation”. British Journal of Pharmacology. 157: pgs 907-921) agree that inter-species dose extrapolation is generally poorly understood. Ultimately, though potentially therapeutic drug doses in humans can be hypothesized and approximated from doses in rats, they are necessarily found by trial and error. Below is a brief and non-exhaustive description of the differing pharmacological characteristics of the two dual orexin receptor antagonists (DORAs) used. Their many differences, combined with both of their therapeutic effects in treating SAD symptoms in the Wistar model, point to the effectiveness of down-regulating the orexin system for therapeutic treatment of seasonality.

Certain pharmacological properties of the orexin receptor antagonists, almorexant and TCS 1102, which were employed in the exemplary research are presented in the below chart.

| | TCS 1102 | Almorexant HCl |
|---|---|---|
| Property | (Bergman et al. 2008) | (Malherbe et al. 2009) |
| Human OxR2 Ki (nM) | 0.2 | 0.9 |
| Human OxR1 Ki (nM) | 3 | 4.7 |
| Human OxR2 FLIPR (nM) | 4 | 15.6 |
| Human OxR1 RLIPR (nM) | 17 | 24.1 |

Bergman et al. 2008. “Proline bis-amides as potent dual orexin receptor antagonists”. Bioorganic & Medicinal Chemistry Letters. 18: pgs 1425-1430.
Malherbe et al. 2009. “Biochemical and Electrophysiological Characterization of Almorexant, a Dual Orexin 1 Receptor (OX1)/Orexin 2 Receptor (OX2) Antagonist: Comparison with Selective OX1 and OX2 Antagonists”. Molecular Pharmacology. 76: pgs 618-631.

Orexin receptors 1 and 2 are known to be highly conserved across all mammalian species, making rat receptor kinetics translational to humans and vice versa (Kaminski and Smolinska et al. 2012. “Chapter Four—Expression of Orexin Receptors in the Pituitary”. Sleep Hormones. 89: pgs 61-73). Furthermore, while Bergman et al. (Bergman et al. 2008. “Proline bis-amides as potent dual orexin receptor antagonists”. Bioorganic & Medicinal Chemistry Letters. 18: pgs 1425-1430) demonstrated that TCS 1102's rat in vivo half life is around 0.3 hours, Callander et al. (Callander et al. 2013. “Kinetic properties of “dual” orexin receptor antagonists at OX1R and OX2R orexin receptors”. Frontiers in Neuroscience. 7: article 230, pgs 1-10) noted that Almorexant's Ki in rat OxR1 cell cultures remains relatively unchanged from t=30 min to t=4 hours, and that OxR2 Ki values decrease over 4 hours. A decreasing Ki means that lower concentrations of the drug can antagonize approximately half of the receptors. Therefore, in the limited amount of research that has been done on TCS 1102, a relatively short half-life has been determined in rats. Though equivalent studies have not been done for Almorexant, its decreasing Ki values over 4 hours suggest that it acts on a much slower timescale at the OxR2 than TCS 1102 does.

TCS 1102 and Almorexant have different affinities for OxR1 and OxR2. TCS 1102 is 15× more selective for human OxR2 while Almorexant is 5× more selective for human OxR2. This is not an exhaustive exploration of the differences between the pharmacology of TCS 1102 and Almorexant; it serves to indicate that these drugs (active agents) have different receptor antagonisms, pharmacokinetics, and time courses. This shows that effect of being an orexin receptor antagonist is the therapeutic characteristic of these active agents. Other agents and drugs being orexin receptor antagonists would have a similar therapeutic effect.

Based upon the pharmacological properties of TCS 1102, proline bis-amide dual orexin receptor antagonists can be administered for the treatment of SAD in humans.

One of the two dual orexin receptor antagonists tested in the exemplary research was TCS 1102, a proline bis-amide DORA. This exemplary research has shown that the following antagonistic profile of TCS 1102 provided the therapeutic effect:

| | | Optomized Proline |
|---|---|---|
| | TCS 1102 (Bergman | bis-amide |
| Property | et al. 2008) | inhibitor 19 |
| Human OxR2 Ki (nM) | 0.2 | 0.8 |
| Human OxR1 Ki (nM) | 3 | 15 |
| Human OxR2 FLIPR (nM) | 4 | 5 |
| Human OxR1 RLIPR (nM) | 17 | 98 |
| P-gp (B to A/A to B) | 1.4 | 2 |
| Log P | 3.4 | 2.6 |
| Rat PK (Cl) mL/min/kg | 3.7 | 35 |
| Rat PK (t½) | 0.3 h | 0.3 h |
| Rat PK (% F) | 11 | 23 |
| Brain/plasma/CSF (nM) | 2370/3500/43 | 13007/25705/860 |
| 100 mpk ip 30 min | | |
| Panlabs IC50 | 3 hits < 10 micro M | 2 hits < 10 micro M |

Furthermore, the previously cited Bergman et al. 2008 demonstrated that different variations in molecular structure of proline bis-amides have comparable pharmacological properties. Therefore, demonstrating the effectiveness of TCS 1102 indicated the effectiveness of all proline bis-amide DORAs.

The active agents of the present invention are orexin receptor antagonists that are administered in therapeutically effective amounts to reduce the symptoms of SAD. The agents include both dual and single orexin receptor antagonists, and further include for example (see research Examples) almorexant HCL, TCS 1102 and proline bis-amide dual orexin receptor antagonists. It is noted, however, that the specific research grade DORAs used in the exemplary research are research grade, and pending human clinical trials, may not necessarily be good agents of orexin antagonism in humans. Rather, they serve to indicate the effectiveness of all compounds that selectively target the orexin receptors. The active agents can be administered by any conventional means available for use in conjunction with pharmaceuticals, either as individual therapeutic agents or in a combination of therapeutic agents. For example, administration can be parenteral, i.e., subcutaneous, intravenous, intramuscular, or intraperitoneal, or by oral, rectal or topical administration. They can be administered alone, but are generally administered with a pharmaceutically acceptable carrier selected on the basis of the chosen route of administration and standard pharmaceutical practice. An “effective” amount or a “therapeutically effective amount” of an agent that downregulates orexin activity is a nontoxic but sufficient amount of an agent to provide the desired effect. For example, a desired effect would be preventing the onset, or reducing the severity, frequency or duration of symptoms associated with SAD. As previously discussed, the amount that is “effective” will vary from subject to subject, depending on the age and general condition of the individual, mode of administration, and the like. While it is not always possible to specify an exact “effective amount,” an appropriate “effective” amount in any individual case may be determined by one of ordinary skill in the art using routine experimentation.

The dosage administered will be dependent on the age, health and weight of the recipient, the extent of disease, kind of concurrent treatment, if any, frequency of treatment, and the nature of the effect desired. Acknowledging the previously discussed errors introduced by scaling up from laboratory rats, for example the orexin receptor antagonists may have a dose range of preferably 60-140 mg/kg, more preferably 80-120 mg/kg, and most preferably, 90-110 mg/kg, regardless of administration route based upon almorexant, and based upon TCS 1102 may have a range of preferably 12-28 mg/kg, more preferably 16-24 mg/kg, and most preferably 18-22 mg/kg regardless of administration route.

The active ingredient of the present invention can be administered orally in solid dosage forms, such as capsules, tablets, and powders, or in liquid dosage forms, such as elixirs, syrups, and suspensions. It can also be administered parenterally, in sterile liquid dosage forms. The active agents of the present invention may be in the form of a pharmaceutically acceptable salt or ester.

Gelatin capsules can contain the active ingredient and powdered carriers, such as lactose, starch, cellulose derivatives, magnesium stearate, stearic acid and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration and/or respiratory inhalants preferably contain a water soluble salt of the active ingredient, suitable stabilizing agents, and if necessary buffer substances. Antioxidizing agents such a sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propylparaben, and chlorbutanol.

Suitable pharmaceutical excipients and carriers are described in the Handbook of Pharmaceutical Excipients, 7^{th} Edition, London, Pharmaceutical Press 2012, a standard reference text in this field. Pharmaceutically acceptable carriers include any of the standard pharmaceutical carriers, such as a phosphate buffered saline solution, water, emulsions such as an oil/water or water/oil emulsion, and various types of wetting agents

Useful pharmaceutical dosage-forms for administration of the compounds of this invention can be illustrated as follows:

Capsules: A large number of unit capsules are prepared by filling standard two-piece hard gelatin capsules each with 100 milligrams of lactose, 50 milligrams of cellulose, and 6 milligrams of magnesium stearate.

Soft Gelatin Capsules: A mixture of active ingredient in a digestible oil such as soybean oil, cottonseed oil or olive oil is prepared and injected by means of a positive displacement pump into gelatin to form gelatin capsules containing 100 milligrams of the active ingredient. The capsules are washed and dried.

Tablets: A large number of tablets are prepared by conventional procedures so that the dosage unit is 100 milligrams of active ingredient, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption. The active ingredient is typically mixed with pharmaceutical carriers, excipients and/or fillers.

Rectal Suppository: Suppositories may be made from a fatty base in which the active ingredients and other excipients are dissolved. A typical fatty base is cocoa butter. Additional suppositories are made from a water soluble base, including polyethylene glycol. Suppositories may also be glycerin based, for example, made of glycerol and gelatin.

Injectable: A parenteral composition suitable for administration by injection is prepared by stirring 1.5% by weight of active ingredient in 10% by volume propylene glycol. The solution is made to volume with water for injection and sterilized.

Suspension: An aqueous suspension is prepared for oral administration so that each 5 milliliters contain 100 milligrams of finely divided active ingredient, 100 milligrams of sodium carboxymethyl cellulose, 5 milligrams of sodium benzoate, 1.0 grams of sorbitol solution, U.S.P., and 0.25 milliliters of vanillin.

Topical: The topical compositions are advantageously in the form of ointments, salves, tinctures, creams, emulsions, solutions, lotions, sprays, powders, gels, suspensions, patches or saturated pads. The compounds are mixed with inert, nontoxic, generally liquid or pasty bases which are suitable for treatment by a topical route with concentrations of active compound(s) ranging from 0.0005% to 5% by weight.

Cosmetic: The present invention also provides a cosmetic composition containing, in a cosmetically acceptable carrier, at least one compound, or its salts or isomers, the composition being, in particular, in the form of a lotion, gel, cream, soap or shampoo.

In addition to the ingredients mentioned above, including active compounds and agents of the present invention, the formulations of the invention may include other agents conventional in the art in reference to the type of formulation, e.g., those suitable for oral administration may include such further agents such as sweeteners, flavoring agents, thickeners, etc. The formulations of the invention for human or veterinary use may be presented in unit-dose or multi-dose sealed containers (ampoules and vials) and may be stored in a lyophilyzed or freeze-dried form requiring the addition of a sterile liquid carrier for reconstitution immediately prior to use.

Another embodiment of the present invention is a diagnostic analysis for SAD treatment comprising a method of determining whether an individual is responsive to a treatment for seasonal affective disorder, the method including determining the levels of orexin in a biological sample from the individual obtained once in the spring or summer and once during the fall or winter prior to said treatment, and determining a difference in level of orexin; administering said treatment for period of time to down regulate orexin receptor activity; determining the levels of orexin in a biological sample from the individual during or after said treatment period, comparing the levels of orexin present in the sample obtained after initiating treatment to a baseline level of orexin present in the sample obtained prior to treatment during spring or summer, and determining that the individual is responsive to treatment if orexin levels are reduced during or after said treatment period.

Currently there are many different questionnaires that attempt to quantify seasonality in humans, including but not limited to the SPAQ, Seasonal Pattern Assessment Questionaire, and the GSS, or Global Seasonality Score. Numerous reports have found that these questionnaires over diagnosed populations tested with SAD compared to what would be diagnosed in person by a doctor. Because orexin level increases in the fall and winter underlie the behavioral profile of SAD in rats, SAD in humans may be diagnosed by both the presence of symptoms and increased orexin activity. Increased orexin activity can be tested in many biological specimens, including in cerebrospinal fluid samples and blood samples. By testing SAD patients for orexin levels when symptoms are not present in the spring and summer and again in the fall and winter when symptoms present themselves, the test enables the tailoring of drug doses of orexin down regulators to decrease orexin levels back to summer levels. Testing for seasonal orexin changes in SAD patients and tailoring a drug regimen to revert fall and winter levels of orexin back to spring and summer levels will enable greater care for unwanted seasonality in humans.

Seasonality in the presentation of bipolar disorder has been well documented in numerous studies. Akhter et al. (Akhter et al. 2013. “Seasonal variation of manic and depressive symptoms in bipolar disorder”. Bipolar Disorders. 15: 377-384) examined 112 patients diagnosed with bipolar II disorder and found a statistically significant decrease in manic/hypomanic/mixed episodes around May, and a statistically significant increase in episodes around August. Episodes in August were roughly 2× higher than the norm. The exemplary research demonstrates that SL photoperiods cause symptoms in rats that can be treated completely with orexin receptor antagonists (ORAs). Accordingly, decreasing photoperiods are theorized to cause increases in orexin activity, and increasing photoperiods are theorized to decrease orexin activity. In the exemplary research, symptoms of seasonality in rats were treated with dual orexin receptor antagonists (DORAs) starting 6 weeks after the photoperiod manipulation began, similar to how Akhter et al. found a significant increase in bipolar II episodes roughly 6 weeks after the summer solstice. Choi et al. (Choi et al. 2010. “Association of seasonality and premenstrual symptoms in Bipolar I and Bipolar II disorders”. Journal of Affective Disorders. 129: 313-316) found similar results. Choi et al. tested 61 women with bipolar I or bipolar II disorder in the Global Seasonality Scale (GSS), against a healthy control group. Both bipolar I and bipolar II patients groups had statistically significant increases in GSS scores with p values compared to controls of 0.001 and less than 0.0001 respectively. Choi et al. collaborated Akhter et al.'s findings of increased seasonal symptoms specifically in bipolar II patient populations.

Friedman et al. (Friedman et al. 2006. “Seasonal changes in clinical status in bipolar disorder: a prospective study in 1000 STEP-BD patients”. Acta Psychiatrica Scandinavica) corroborate these studies as well. This paper examined the change in prevalence of 5 types of symptoms of bipolar disorder in 1000 patients. Increases in depression symptoms were found from July to October in bipolar patients. Goikolea et al. (Goikolea et al. 2007. “Clinical and prognostic implications of seasonal pattern in bipolar disorder: a 10-year follow-up of 302 patients”. Psycological Medicine. 37: 1595-1599) performed a 10 year study on 302 patients with bipolar disorder. The timeframe of this study lends more credence to its results, as comparable studies are generally only one year long. Of the bipolar patients in the study, 23.7% presented with seasonally varying symptom profiles. The most generous seasonality questionnaires do not generally find significant seasonality in more than 10% of healthy controls living at seasonal latitudes. Goikolea et al. found that bipolar patients with seasonality had significantly higher rates of hypomanic episodes, which may be modeled by the exemplary research's marble burying tests. Increased prevalence of depressive episodes was also found in the seasonal bipolar population. The seasonal bipolar population was also significantly more likely to have a predominantly depressive bipolar disorder classification. This study also found more seasonal symptoms in bipolar II disorder than bipolar I disorder. Shand et al. (Shand et al. 2011. “The seasonality of bipolar affective disorder: Comparison with a primary care sample using the Seasonal Pattern Assessment Questionnaire”. Journal of Affective Disorders. 132: 289-292) found that bipolar patients score significantly higher than controls in the GSS and the Seasonal Pattern Assessment Questionaire (SPAQ). Shin et al. (Shin et al. 2005. “Seasonality in a community sample of bipolar, unipolar and control subjects”. Journal of Affective Disorders. 86: 19-25) collaborated, and found that patients with bipolar disorder have statistically significantly more seasonal fluctuations in mood and behavior than patients with non-seasonal depression and healthy controls. In this study, whether or not bipolar disorder patients self-reported a seasonal presentation of symptoms, they scored as high or higher than patients with self-reported seasonal depression in every sub-section of the GSS. This confirms that the bipolar population suffers from a greater than normal prevalence of seasonality.

Finally, confirming all studies previously reported here, Simonsen et al. (Simonsen et al. 2011. “Seasonal symptoms in bipolar and primary care patients”. Journal of Affective Disorders. 132: 200-208) found increased seasonal mood fluctuations in bipolar patients when compared to other patients consulting primary care physicians. This study scored 183 patients with bipolar disorder and 468 patients in primary care for other reasons with the SPAQ. Strikingly, bipolar patients reported seasonal changes in social activity significantly different from the controls with a p value less than 0.001. Bipolar patients were also found to sleep on average 1.8 hours more during the winter than in the summer. Ultimately, 46% of bipolar patients reported seasonal changes in overall well-being to be a “moderate” problem.

These studies, like all studies, have confounding factors. For example, true controls to patients with bipolar disorder may be impossible to recreate. Most studies are naturalistic in that they don't require that bipolar patients in the study stop all medications, which may mask seasonal symptoms. Despite confounding factors, the collaboration between these studies elucidates seasonality as a significant aspect of bipolar disorder, specifically bipolar II disorder. The exemplary research, explained in the below Examples, has elucidated orexin receptor antagonists (ORAs) as a class of active agents and compounds that effectively treat physiological and behavioral seasonality, and therefore reduces seasonal symptoms of bipolar disorder.

Thus, another embodiment of the present invention is directed to a method for reducing one or more seasonal symptoms associated with bipolar disorder in an individual, the method comprising administering to the individual suffering from seasonal symptoms associated with bipolar disorder, a composition comprising a therapeutically effective amount of an agent to down regulate the activity of an orexin selected from the group consisting of orexin A or orexin B, at one or more orexin receptors.

Seasonal effects on symptoms of cycling ovulation, also known as Pre Menstrual Syndrome or PMS, have been demonstrated in humans. Maskall et al. (Maskall et al. 1997. “Seasonality of symptoms in women with late luteal phase dysphoric disorder”. American Journal of Psychiatry. 154: pgs 1436-1441 found that 70% of women have fewer PMS symptoms in the summer, cited unpublished data showing that 51% of 200 women with SAD complained of substantial PMS symptoms, and found that in a patient population of 100 women with late luteal phase dysphoric disorder, 38% met SAD diagnosis criteria compared with only 8% of the control group. Furthermore, Choi et al. (Choi et al. 2011. “Association of seasonality and premenstrual symptoms in Bipolar I and Bipolar II disorders”. Journal of Affective Disorders. 129: pgs 313-316) noted an “associat[tion] of seasonality and premenstrual symptoms in Bipolar I and Bipolar II disorders”. Lam et al. (Lam et al. 1997. “A controlled study of light therapy in women with late luteal phase dysphoric disorder”. Psychiatry Research. 86: pgs 185-192) found that light therapy was moderately effective in treating women with symptoms of late luteal phase dysphoric disorder.

As the exemplary research shows that antagonism of the orexin receptor is a very effective way to treat symptoms of seasonality in female Wistar rats with intact ovulatory cycling, and given the inefficiency of treating symptoms of seasonality with light therapy, a claim is made for the treatment of seasonal PMS symptom worsening in women with orexin receptor antagonism.

Photoperiod studies are overwhelmingly performed on male Wistar rats, as opposed to female Wistar rats, as females can display behavioral profiles from ovulatory cycling (Molina-Hernandez and Tellez-Alcantara (Molina-Hernandez and Tellez-Alcantara 2000. “Long Photoperiod Regimen may Produce Antidepressant Actions in the Male Rat”. Progress in Neuro-Psychopharmacology & Biological Psychiatry. Vol. 24. Issue 1. pgs 105-116); Prendergast and Kay (Prendergast and Kay 2008. “Affective and Adrenocorticotrophic Responses to Photoperiod in Wistar Rats”. Journal of Neuroendocrinology. 20. pgs 261-267)). Despite this, in humans, the greater burden of seasonality symptoms is on women, not men (Suhail and Cochrane (Suhail and Cochrane 1998. “Seasonal variations in hospital admission for affective disorders by gender and ethnicity”. Social Psychiatry and Psychiatric Epidemiology. 33: pgs 211-217). Accordingly, the experiments in the exemplary research utilized only female Wistar rats whose ovulatory cycling was left intact.

As previously referenced, females having SAD tend to suffer from more symptoms of pre-menstrual syndrome (PMS), and PMS symptoms may be seasonal in severity. Inasmuch as PMS symptoms vary in severity between spring-summer and fall-winter, drugs that decrease the orexin system can be therapeutic by reducing symptoms.

Thus, another embodiment of the present invention is directed to a method for reducing one or more seasonal symptoms associated with premenstrual syndrome in an individual, the method comprising administering to the individual suffering from seasonal symptoms associated with premenstrual syndrome, a composition comprising a therapeutically effective amount of an agent to down regulate the activity of an orexin selected from the group consisting of orexin A or orexin B, at one or more orexin receptors.

Thus, the invention is demonstrated with reference to the following examples, which are of an illustrative nature only and which are to be construed as non-limiting.

### Examples

Seasonal affective disorder is a major depressive disorder recurring in the fall and winter. It is caused by the reduction of light in the environment. Past studies have shown that a shortening of the daylight duration induced depressive-like and anxiety-like symptoms in diurnal rodents (Ashkenazy T, Einat H, Kronfeld-Schor N. Effects of bright light treatment on depression- and anxiety-like behaviors of diurnal rodents maintained on a short daylight schedule. Behav Brain Res. 2009; 201:343-346) or that a housing in total darkness for 5-6 weeks induced a depressive-like state in nocturnal rats (Gonzalez MM, Aston-Jones G. Light deprivation damages monoamine neurons and produces a depressive behavioral phenotype in rats. Proc Natl Acad Sci USA. 2008; 105:4898-4903). On the other hand, long photoperiod has been shown to produce antidepressant-like effect in nocturnal rats (Molina-Hernandez M, Téllez-Alcántara P. Long photoperiod regimen may produce antidepressant actions in the male rat. Prog Neuropsychopharmacol Biol Psychiatry. 2000; 24:105-116).

The first goal of the present Example (Part 1) was to investigate the effects of progressive changes—shortening and lengthening—of the daylight duration. Animals (female Wistar rats) were housed for a 8-week period in three conditions: constant 12 h/12 h light/dark cycle, daily 8-min shortening of the daylight durations and daily 8-min lengthening of the daylight durations. These effects were examined by using different tests: the open-field test (Hiroi R, Neumaier J F. Estrogen decreases 5-HT1B autoreceptor mRNA in selective subregion of rat dorsal raphe nucleus: inverse association between gene expression and anxiety behavior in the open field. Neuroscience. 2009; 158:456-464; and Prut L, Belzung C. The open field as a paradigm to measure the effects of drugs on anxiety-like behaviors: a review. Eur J Pharmacol. 2003; 463:3-33), the elevated-plus-maze test (Belzung C, Griebel G. Measuring normal and pathological anxiety-like behaviour in mice: a review. Behav Brain Res. 2001; 125:141-149; and Pellow S and File SE, Anxiolytic and anxiogenic drug effects on exploratory activity in an elevated plus-maze: a novel test of anxiety in the rat. Pharmacol Biochem Behav. 1986; 24:525-529) and the marble burying test (Broekkamp C L, Rijk H W, Joly-Gelouin D, Lloyd K L. Major tranquillizers can be distinguished from minor tranquillizers on the basis of effects on marble burying and swim-induced grooming in mice. Eur J Pharmacol. 1986; 126:223-229; and Joel D. Current animal models of obsessive compulsive disorder: a critical review. Prog Neuropsychopharmacol Biol Psychiatry. 2006; 30:374-388), which evaluate anxiety-like behavior, and the forced swimming test which evaluates depression-like behavior (Porsolt R D, Bertin A, Jalfre M. “Behavioural despair” in rats and mice: strain differences and the effects of imipramine. Eur J Pharmacol. 1978; 51:291-294; and Thiébot M H, Martin P, Puech A J. Animal behavioural studies in the evaluation of antidepressant drugs. Br J Psychiatry Suppl. 1992 February; (15):44-50). It was expected that the progressive shortening of the daylight duration would induce both a depressive behavioral phenotype and an increase in anxiety-like behavior, and that conversely, the progressive lengthening of the daylight duration would induce both an antidepressant-like effect and an anxiolytic-like effect.

The second goal of this Example (Part 2) was to test the effect of two dual orexin receptor antagonists, almorexant HCL and TCS 1102, on potential behavioral effects of the daylight shortening. For this purpose, the animals subjected to the daylight duration shortening received a daily administration of almorexant (100 mg/kg, per-os: PO), TCS 1102 (20 mg/kg, IP) or their vehicles from the 5^{th} week and until the 8^{th} week of the daylight shortening periods.

Rats were weighed three times a week in order to examine the effect of L/D changes and the effects of treatments on the body weight.

### Materials and Methods

General Points, Ethical Concerns

Manipulations of animals were conducted carefully in order to minimize stress. All the experiments were performed within the guidelines of the French Ministry of Agriculture for experiments with laboratory animals (law 2013-118) as the study was contracted to a French contract research organization.

Animals were euthanatized at the end of the study by an intraperitoneal injection of sodium pentobarbital (200 mg/kg). Euthanasia was performed in compliance with the guidelines of the French Ministry of Agriculture for experiments with laboratory animals (law 2013-118).

The experimental protocol and euthanasia have been approved by the Ethical Committee N^{o} 27, registered at the French ministry of research.

Experiments were conducted between 8 h 30 and 13 h 30 in standard conditions (T°=22.0±1.5° C.) with artificial light in quiet conditions (no noise except those generated by ventilation and by the apparatus used for experiments).

The animals were not subjected to other experiments before the study.

Each animal was identified with a bar code marked on the tail.

Experiments were analyzed by a technician blind to the treatment.

Animals

1. Wistar rats.

2. Number of animals: 60.

3. Origin (breeder): Elevage R. Janvier, France.

4. Gender: female.

5. Age
a. At the arrival at the laboratory: 4-week old.
b. Age at the beginning of the modification of the light/dark duration: 6-week old.
c. At the beginning of drugs administrations: 10-week old.
d. At the time of the tests:
   i. Open-Field 1^{st} session, FST 1^{st} session: 11-week old.
   ii. EPM, Open-Field 2^{nd} session: 12-week old.
   iii. Marble burying test, FST 2^{nd} session: 13-week old.
   iv. Blood and brain samples collection: 14-week old.

Housing, Feeding

1. Type E cages (Iffa Credo, L'Arbresle, France) in polycarbonate (45 cm deep; 30 cm large; 20 cm high, area=1032 cm²) covered with a stainless steel grid in which food and a bottle are placed. A stainless steel removable divider separated food and water.

2. Litter: Aspen Small (SDS Dietex, France).

3. Number of animals/cage: 3.

4. Temperature: 22.0±1.5° C.

5. Hygrometry: 50±25%.

6. Animals were housed in rooms S3 (C condition), S1 (LL condition) and S2 (SL condition) of the animal facility, in which no other animals were housed (C, LL and SL conditions)

7. Lighting: 100 Lux; light/dark cycle)

8. Food rat-mouse A04 (Safe, France), available ad libitum.

9. Drink: Tap water, available ad libitum.

### Drugs

| Almorexant HCl | Certified analysis. |
|---|---|
| Supplier | Greenpharma, France |
| Batch no. | P3187928 |
| Formula | C₂₉H₃₂ClF₃N₂O₃ |
| Molecular weight | Almorexant: 513 - Almorexant HCl: 549 |
| Preparation | Dissolved in distilled water, aliquoted. |
| | Aliquots were stored at −80° C. and |
| | were defrosted before use |
| Storage condition | Less than 6 h, ambient temperature |
| before use | (22-23° C.), protect from light |
| Appearance of the | Transparent, completely dissolved |
| solution | |
| pH of the solution | 4 |
| Administration route | Per-os gavage (PO) |
| Dose(s) | 100 mg/kg |
| Correction factor | 1.07 |
| Application volume | 2 ml/kg |
| Number of | 29 (1 per day for 29 days) |
| administrations | |
| Time of administration | Between 9:30 and 12:00 or 1 h prior test |
| TCS 1102 | Certified analysis |
| Supplier | Greenpharma, France |
| Batches no. | P7522747 |
| Formula | C₂₇H₂₆N₄O₂S |
| Molecular weight | 571 |
| Preparation | Dissolved in 20% Dimethyl sulfoxide |
| | (DMSO)/80% distilled water, aliquoted. |
| | Aliquots were stored at −80° C. |
| | and were defrosted before use |
| Storage condition | Less than 6 h, ambient temperature |
| before use | (22-23° C.), protect from light |
| Appearance of the | Opaque white suspension |
| solution | |
| pH of the solution | 7 |
| Administration route | Intraperitoneal (IP) injection |
| Dose(s) | 20 mg/kg |
| Correction factor | 1 |
| Application volume | 2 ml/kg |
| Number of | 29 (1 per day for 29 days) |
| administrations | |
| Time of administration | Between 9:30 and 12:00 or 1 h prior test |

### Experimental Design

The planning of the study is presented in the following Table 1 and Table 2.

The light duration was 12 h light/12 dark during the first 13 days (d-13 to d-01, Table 1).

Animals were divided into three groups housed under three L/D conditions:
1) C condition (Control; N=12): the L/D cycle remained 12 h light/12 h dark during all the experiment.
2) LL condition (Long Light; N=12): the light duration progressively increased by 8 min every day (+4 min at the beginning of the light period, +4 min at the end of the light period) from d01 to d57.
3) SL condition (Short Light; N=36): the light duration progressively decreased by 8 min every day (−4 min at the beginning of the light period, −4 min at the end of the light period) from d01 to d57.

The three groups were housed in three different rooms. Only animals used for this study were housed in these rooms.

Within each group, animals were then divided into subgroups which received different treatments from d29 until d57, as presented below:

### Subgroups

| | | | Treatments - |
|---|---|---|---|
| L/D condition | | Subgroups | Administration |
| (N) | Groups (N) | (N) | routes |
| C: Control (12) | C-Veh (12) | C-DMSO (6) | 20% DMSO IP |
| | | C-Wat (6) | Distilled water PO |
| LL: Long Light | LL-Veh (12) | LL-DMSO (6) | 20% DMSO IP |
| (12) | | LL-Wat (6) | Distilled water PO |
| SL: Short Light | SL-Veh (12) | SL-DMSO (6) | 20% DMSO IP |
| (36) | | SL-Wat (6) | Distilled water PO |
| | | SL-TCS (12) | TCS (20 mg/kg) IP |
| | | SL-Almo (12) | Almorexant |
| | | | (100 mg/kg) PO |

Treatments were administered every day between 9:30 and 12:00 on the days where no test was conducted, or 1 h prior the test.

Animals were subjected to behavioral tests at different points, as indicated in below Table 2.

Animals' body weight was recorded three days per week. The body weight on day d-4 was considered as the initial body weight.

TABLE 1
Planning of the experiment (Part 1). Days: d−13: receipt of animals; d01:
beginning of L/D changes.

| | Age | Day | | | | | | | | | | |
|---|---|---|---|---|---|---|---|---|---|---|---|---|
| Week | in | L/D | | C group | | | LL group | | | SL group | | |
| Nb | week | change | Treatment | L duration | L On | L Off | L duration | L On | L Off | L duration | L On | L Off |
| −2 | 4 | d −13 | | 12:00 | 05:00 | 17:00 | 12:00 | 05:00 | 17:00 | 12:00 | 05:00 | 17:00 |
| | | d −12 | | 12:00 | 05:00 | 17:00 | 12:00 | 05:00 | 17:00 | 12:00 | 05:00 | 17:00 |
| | | d −11 | | 12:00 | 05:00 | 17:00 | 12:00 | 05:00 | 17:00 | 12:00 | 05:00 | 17:00 |
| | | d −10 | | 12:00 | 05:00 | 17:00 | 12:00 | 05:00 | 17:00 | 12:00 | 05:00 | 17:00 |
| | | d −09 | | 12:00 | 05:00 | 17:00 | 12:00 | 05:00 | 17:00 | 12:00 | 05:00 | 17:00 |
| | | d −08 | | 12:00 | 05:00 | 17:00 | 12:00 | 05:00 | 17:00 | 12:00 | 05:00 | 17:00 |
| −1 | 5 | d −07 | | 12:00 | 05:00 | 17:00 | 12:00 | 05:00 | 17:00 | 12:00 | 05:00 | 17:00 |
| | | d −06 | | 12:00 | 05:00 | 17:00 | 12:00 | 05:00 | 17:00 | 12:00 | 05:00 | 17:00 |
| | | d −05 | | 12:00 | 05:00 | 17:00 | 12:00 | 05:00 | 17:00 | 12:00 | 05:00 | 17:00 |
| | | d −04 | | 12:00 | 05:00 | 17:00 | 12:00 | 05:00 | 17:00 | 12:00 | 05:00 | 17:00 |
| | | d −03 | | 12:00 | 05:00 | 17:00 | 12:00 | 05:00 | 17:00 | 12:00 | 05:00 | 17:00 |
| | | d −02 | | 12:00 | 05:00 | 17:00 | 12:00 | 05:00 | 17:00 | 12:00 | 05:00 | 17:00 |
| | | d −01 | | 12:00 | 05:00 | 17:00 | 12:00 | 05:00 | 17:00 | 12:00 | 05:00 | 17:00 |
| 1 | 6 | d 01 | | 12:00 | 05:00 | 17:00 | 12:00 | 05:00 | 17:00 | 12:00 | 05:00 | 17:00 |
| | | d 02 | | 12:00 | 05:00 | 17:00 | 12:08 | 04:56 | 17:04 | 11:52 | 05:04 | 16:56 |
| | | d 03 | | 12:00 | 05:00 | 17:00 | 12:16 | 04:52 | 17:08 | 11:44 | 05:08 | 16:52 |
| | | d 04 | | 12:00 | 05:00 | 17:00 | 12:24 | 04:48 | 17:12 | 11:36 | 05:12 | 16:48 |
| | | d 05 | | 12:00 | 05:00 | 17:00 | 12:32 | 04:44 | 17:16 | 11:28 | 05:16 | 16:44 |
| | | d 06 | | 12:00 | 05:00 | 17:00 | 12:40 | 04:40 | 17:20 | 11:20 | 05:20 | 16:40 |
| | | d 07 | | 12:00 | 05:00 | 17:00 | 12:48 | 04:36 | 17:24 | 11:12 | 05:24 | 16:36 |
| 2 | 7 | d 08 | | 12:00 | 05:00 | 17:00 | 12:56 | 04:32 | 17:28 | 11:04 | 05:28 | 16:32 |
| | | d 09 | | 12:00 | 05:00 | 17:00 | 13:04 | 04:28 | 17:32 | 10:56 | 05:32 | 16:28 |
| | | d 10 | | 12:00 | 05:00 | 17:00 | 13:12 | 04:24 | 17:36 | 10:48 | 05:36 | 16:24 |
| | | d 11 | | 12:00 | 05:00 | 17:00 | 13:20 | 04:20 | 17:40 | 10:40 | 05:40 | 16:20 |
| | | d 12 | | 12:00 | 05:00 | 17:00 | 13:28 | 04:16 | 17:44 | 10:32 | 05:44 | 16:16 |
| | | d 13 | | 12:00 | 05:00 | 17:00 | 13:36 | 04:12 | 17:48 | 10:24 | 05:48 | 16:12 |
| | | d 14 | | 12:00 | 05:00 | 17:00 | 13:44 | 04:08 | 17:52 | 10:16 | 05:52 | 16:08 |
| 3 | 8 | d 15 | | 12:00 | 05:00 | 17:00 | 13:52 | 04:04 | 17:56 | 10:08 | 05:56 | 16:04 |
| | | d 16 | | 12:00 | 05:00 | 17:00 | 14:00 | 04:00 | 18:00 | 10:00 | 06:00 | 16:00 |
| | | d 17 | | 12:00 | 05:00 | 17:00 | 14:08 | 03:56 | 18:04 | 9:52 | 06:04 | 15:56 |
| | | d 18 | | 12:00 | 05:00 | 17:00 | 14:16 | 03:52 | 18:08 | 9:44 | 06:08 | 15:52 |
| | | d 19 | | 12:00 | 05:00 | 17:00 | 14:24 | 03:48 | 18:12 | 9:36 | 06:12 | 15:48 |
| | | d 20 | | 12:00 | 05:00 | 17:00 | 14:32 | 03:44 | 18:16 | 9:28 | 06:16 | 15:44 |
| | | d 21 | | 12:00 | 05:00 | 17:00 | 14:40 | 03:40 | 18:20 | 9:20 | 06:20 | 15:40 |
| 4 | 9 | d 22 | | 12:00 | 05:00 | 17:00 | 14:48 | 03:36 | 18:24 | 9:12 | 06:24 | 15:36 |
| | | d 23 | | 12:00 | 05:00 | 17:00 | 14:56 | 03:32 | 18:28 | 9:04 | 06:28 | 15:32 |
| | | d 24 | | 12:00 | 05:00 | 17:00 | 15:04 | 03:28 | 18:32 | 8:56 | 06:32 | 15:28 |
| | | d 25 | | 12:00 | 05:00 | 17:00 | 15:12 | 03:24 | 18:36 | 8:48 | 06:36 | 15:24 |
| | | d 26 | | 12:00 | 05:00 | 17:00 | 15:20 | 03:20 | 18:40 | 8:40 | 06:40 | 15:20 |
| | | d 27 | | 12:00 | 05:00 | 17:00 | 15:28 | 03:16 | 18:44 | 8:32 | 06:44 | 15:16 |
| | | d 28 | | 12:00 | 05:00 | 17:00 | 15:36 | 03:12 | 18:48 | 8:24 | 06:48 | 15:12 |

TABLE 2
Planning of the study (Part 2). Days: d29: beginning of treatments; d57: blood
and brain samples collection, sacrifice of animals.

| | Age | Day | | | | | | | | | | | |
|---|---|---|---|---|---|---|---|---|---|---|---|---|---|
| | in | L/D | | C group | | | LL group | | | SL group | | | |
| Week Nb | week | change | Treatment | L duration | L On | L Off | L duration | L On | L Off | L duration | L On | L Off | Tests |
| 5 | 10 | d 29 | t 1 | 12:00 | 05:00 | 17:00 | 15:44 | 03:08 | 18:52 | 8:16 | 06:52 | 15:08 | |
| | | d 30 | t 2 | 12:00 | 05:00 | 17:00 | 15:52 | 03:04 | 18:56 | 8:08 | 06:56 | 15:04 | |
| | | d 31 | t 3 | 12:00 | 05:00 | 17:00 | 16:00 | 03:00 | 19:00 | 8:00 | 07:00 | 15:00 | |
| | | d 32 | t 4 | 12:00 | 05:00 | 17:00 | 16:08 | 02:56 | 19:04 | 7:52 | 07:04 | 14:56 | |
| | | d 33 | t 5 | 12:00 | 05:00 | 17:00 | 16:16 | 02:52 | 19:08 | 7:44 | 07:08 | 14:52 | |
| | | d 34 | t 6 | 12:00 | 05:00 | 17:00 | 16:24 | 02:48 | 19:12 | 7:36 | 07:12 | 14:48 | |
| | | d 35 | t 7 | 12:00 | 05:00 | 17:00 | 16:32 | 02:44 | 19:16 | 7:28 | 07:16 | 14:44 | |
| 6 | 11 | d 36 | t 8 | 12:00 | 05:00 | 17:00 | 16:40 | 02:40 | 19:20 | 7:20 | 07:20 | 14:40 | |
| | | d 37 | t 9 | 12:00 | 05:00 | 17:00 | 16:48 | 02:36 | 19:24 | 7:12 | 07:24 | 14:36 | Open-Field-Session 1 (30 rats) |
| | | d 38 | t 10 | 12:00 | 05:00 | 17:00 | 16:56 | 02:32 | 19:28 | 7:04 | 07:28 | 14:32 | Open-Field-Session 1 (30 rats) |
| | | d 39 | t 11 | 12:00 | 05:00 | 17:00 | 17:04 | 02:28 | 19:32 | 6:56 | 07:32 | 14:28 | FST-Trial 1-Session 1 |
| | | d 40 | t 12 | 12:00 | 05:00 | 17:00 | 17:12 | 02:24 | 19:36 | 6:48 | 07:36 | 14:24 | FST-Trial 2-Session 1 |
| | | d 41 | t 13 | 12:00 | 05:00 | 17:00 | 17:20 | 02:20 | 19:40 | 6:40 | 07:40 | 14:20 | |
| | | d 42 | t 14 | 12:00 | 05:00 | 17:00 | 17:28 | 02:16 | 19:44 | 6:32 | 07:44 | 14:16 | |
| 7 | 12 | d 43 | t 15 | 12:00 | 05:00 | 17:00 | 17:36 | 02:12 | 19:48 | 6:24 | 07:48 | 14:12 | EPM (20 rats) |
| | | d 44 | t 16 | 12:00 | 05:00 | 17:00 | 17:44 | 02:08 | 19:52 | 6:16 | 07:52 | 14:08 | EPM (20 rats) |
| | | d 45 | t 17 | 12:00 | 05:00 | 17:00 | 17:52 | 02:04 | 19:56 | 6:08 | 07:56 | 14:04 | EPM (20 rats) |
| | | d 46 | t 18 | 12:00 | 05:00 | 17:00 | 18:00 | 02:00 | 20:00 | 6:00 | 08:00 | 14:00 | Open-Field-Session 2 (30 rats) |
| | | d 47 | t 19 | 12:00 | 05:00 | 17:00 | 18:08 | 01:56 | 20:04 | 5:52 | 08:04 | 13:56 | Open-Field-Session 2 (30 rats) |
| | | d 48 | t 20 | 12:00 | 05:00 | 17:00 | 18:16 | 01:52 | 20:08 | 5:44 | 08:08 | 13:52 | |
| | | d 49 | t 21 | 12:00 | 05:00 | 17:00 | 18:24 | 01:48 | 20:12 | 5:36 | 08:12 | 13:48 | |
| 8 | 13 | d 50 | t 22 | 12:00 | 05:00 | 17:00 | 18:32 | 01:44 | 20:16 | 5:28 | 08:16 | 13:44 | |
| | | d 51 | t 23 | 12:00 | 05:00 | 17:00 | 18:40 | 01:40 | 20:20 | 5:20 | 08:20 | 13:40 | Marble (30 rats) |
| | | d 52 | t 24 | 12:00 | 05:00 | 17:00 | 18:48 | 01:36 | 20:24 | 5:12 | 08:24 | 13:36 | Marble (30 rats) |
| | | d 53 | t 25 | 12:00 | 05:00 | 17:00 | 18:56 | 01:32 | 20:28 | 5:04 | 08:28 | 13:32 | FST-Trial 1-Session 2 |
| | | d 54 | t 26 | 12:00 | 05:00 | 17:00 | 19:04 | 01:28 | 20:32 | 4:56 | 08:32 | 13:28 | FST-Trial 2-Session 2 |
| | | d 55 | t 27 | 12:00 | 05:00 | 17:00 | 19:12 | 01:24 | 20:36 | 4:48 | 08:36 | 13:24 | |
| | | d 56 | t 28 | 12:00 | 05:00 | 17:00 | 19:20 | 01:20 | 20:40 | 4:40 | 08:40 | 13:20 | |
| 9 | 14 | d 57 | t 29 | 12:00 | 05:00 | 17:00 | 19:28 | 01:16 | 20:44 | 4:32 | 08:44 | 13:16 | Samples collection |

Tests

The tests were conducted at the middle of the light phase +/−2 h 30 min.

Open-Field Test

The activity in the central part of an open-field is considered as a reliable index of anxiety in rodents.

Animals were subjected to the open-field test at two different times of the study: at the 9^{th} or 10^{th} days of treatment and at the 18^{th} or 19^{th} days of treatment (see Table 2). The test was conducted by a male experimenter referred as VC.

Activity was tested in open boxes (open-fields: 100 cm wide, 100 cm long, 50 cm high) made of PVC. Animals' activity was recorded by an electronic videotracking detection system (Viewpoint, France) allowing to detect the distance travelled and the time spent in different parts of the open-field.

Light intensity was 7-10 Lux at the center of the open-fields.

Animals were individually placed for a 10-min session in the center of one quadrant of the open-field.

Parameters analyzed were:
1) One parameter taken as an index of locomotor-exploratory behavior:
   The distance travelled in the open-field.
2) Two parameters taken as indices of anxiety behavior:
   a) % Dist Cent=the percentage of distance travelled in the central 60×60 cm area of the open-field.
   b) % Time cent=the percentage of time spent in the central 60×60 cm area of the open-field.

Other parameters were recorded: the distance travelled in the 60×60 cm center, the distance travelled in the 20×20 cm the center in cm, the percentage of distance travelled in the 20×20 cm the center and the percentage of time spent in the 20×20 cm the center. The analyses of these parameters are not presented here because they did not provide any additional information.

The reanalyzed parameters from raw research data confirmed the research analyses. New parameters tested were:

1) % Distance in the Periphery: distance spent in periphery of the OFT divided by total rat distance traveled, or outside of the 60×60 cm center

2) % Distance in the Hyper-Center: distance spent in the hyper center divided by individual rat total distance

3) Distance in Hyper-Center: distance traveled in hyper center

4) % Time in Hyper-Center: time spent in hyper-center.

### Elevated-Plus Maze

The elevated-plus maze test is one of the most widely used tests for measuring anxiety-like behavior in rodents.

Animals were subjected to the elevated-plus maze (EPM) test only once during the period of the study: at the 43^{th}, 44^{th} or 45^{th} days of treatment (see Table 2). The test was conducted by a male experimenter referred as VC.

The apparatus consisted of a plus-shaped maze, elevated 50 cm from the floor, with two opposite open arms, 50×10 cm, crossed at right angles by two arms of the same dimensions, but enclosed by 40-cm-high black opaque walls with an open roof. In addition a 1-cm-high edge made of Plexiglas surrounded the open arms to avoid falls.

Light intensity was 10 Lux at the level of open arms.

The experiment was recorded with a camcorder, placed 1.5 m above the maze and connected to a screen, allowing the experimenter to watch the test online.

Animals were individually placed for a 5-min session in the center of the plus-maze. The number of arm entries and the time spent in arms were recorded. An arm entry is defined as all four paws into the arm.

Parameters analyzed were:
1) Two parameters taken as indices of anxiety behavior:
   a) % TOA=the percentage of time spent in open arms=100×‘time spent in open arms’/(time spent in open arms′+‘time spent in enclosed arms’).
   b) % OAE=the percentage of open arm entries=100×‘number of entries in open arms’/(number of entries in open arms′+‘number of entries in enclosed arms’).
2) One parameter taken as an index of locomotor-exploratory behavior:
   AE=the number of arm entries=‘number of entries in open arms’+‘number of entries in enclosed arms’.

Other parameters were recorded: the time spent in open arms, the number of open arm entries, the time spent in enclosed arms, the number of enclosed arm entries and the time spent in the center of the maze. The analyses of these parameters are not presented here because they did not provide any additional information.

Additional reanalysis tested for:

1) Enclosed arm entries: entries into the protected arms of the EPM

### Forced Swimming Test

The forced swimming test (FST) is the most used rodent model for screening antidepressants.

Animals were subjected to the forced swimming test (FST) at two different times of the study: at the 39^{th}-40^{th} day of treatment and at the 53^{rd}-54^{th} day of treatment (see Table 2). The test was conducted by two experimenters: a male experimenter referred as VC and a female experimenter referred as LB. The same animals were tested by the same experimenter on the two FST sessions. The same proportion of animals of each group was tested by each experimenter. In order to minimize the potential impact of the sex of the experimenter on animals' behavior, a tee-shirt worn by LB was placed close to the animals into the experimental room of VC and a tee-shirt worn by VC was placed close to the animals into the experimental room of LB, so that the odor of both experimenters were present in the room.

The apparatus used for the experiment consisted of glass cylinders (64 cm high, 25 cm diameter) filled with 30 cm 25° C. water. The experiment was recorded with a camcorder, placed in front of the glass cylinder and connected to a screen, allowing the experimenter to watch the test online.

Light intensity was 15-20 Lux at the level of glass cylinders.

Each FST session consisted in two trials conducted 24 h apart. On each trial, the animal was dropped in the glass cylinder and remained there for 15 min (first trial) and for 5 min (second trial). The behavior of the rat was measured on the second trial.

Behavior was classified as one of three behaviors:
1) Immobility, defined as floating in the water without struggling and using only small movements to keep the head above water.
2) Swimming, defined as moving limbs in an active manner, more than required to keep the head above water and causing movement among quadrants of the cylinder.
3) Climbing, defined as making active movements with forepaws moving in and out of the water, usually directed against the side of the cylinder.
   Parameters analyzed were:

1) Imo=the immobility time.

2) Swm=the swimming time.

3) Clb=the climbing time.

A depressive-like profile is indicated by an increase of the immobility time and a decrease of either the swimming time or the climbing time. Conversely, an antidepressant-like effect is indicated by a decrease of the immobility time and an increase of either the swimming time or the climbing time.

Reanalysis of raw video footage for the FST provided additional parameters:

1) Climbing Bins: additive sums for total time spent climbing grouped in 5 second bins.

2) Floating Bins: additive sums for total time spent floating in 5 second bins.

### Marble Burying Test

Rodents use bedding material to bury noxious as well as harmless objects. The marble burying test can score the ability of rats to remove slight stressors, the marbles, from their environments. Analyzing the ability to cope with slight stressors and remove them from the environment can measure activity, productiveness, hypomanic bipolar episodes, and have been used in Obsessive Compulsive Modeling. When combined with a photoperiod shifts simulating seasons, the MBT can test for seasonal changes in activity, efficiency, hypomanic bipolar predisposition, and other similar behaviors.

Animals of LL-DMSO, LL-Wat, SL-DMSO, SL-Wat and SL-Almo groups were subjected to the marble burying test only once during the period of the study: at the 51^{st} or 52^{nd} days of treatment (see Table 2). The test was conducted by a male experimenter referred as VC.

The experiment was conducted in twelve Plexiglas transparent open-boxes (42 cm L, 42 cm W, 40 cm H) filled with 5 cm sawdust. Twenty-five clean glass marbles (15 mm diameter) were evenly spaced 5 cm apart on sawdust. The number of marbles buried was counted at the end of the session.

Light intensity was 30 Lux at the center of the open-boxes.

Animals were individually placed for a 5-min session in the experimental apparatus.

The number of marbles was counted at the end of the session:
1) Number of marbles not buried.
2) Number of marbles ⅓ buried.
3) Number of marbles ⅔ buried.
4) Number of marbles totally buried.

Parameters analyzed were:
1) Unburied=the number of marbles not buried.
2) ≧⅔ buried=number of marbles ⅔ buried+number of marbles totally buried
3) Tot buried=number of marbles totally buried.

The analyses of other parameters found similar results:

1) Marbles ≧⅓ buried=marbles ⅓ buried+marbles ⅔ buried+marbles totally buried

2) Marbles ⅓ buried=marbles left unburied+marbles ⅓ buried

3) Marbles ⅓ buried

NB: it was initially planned that the duration of the test was 30 min; pilot experiments showed that most of the marbles were buried after 10 min of test. Therefore, the duration of the test was shortened to 5 min.

### Data Analysis

Data was expressed as mean±standard error of mean (SEM).

Statistical analyses were performed by using Stat View 4.5 and Excel softwares.

### OFT, EPM and FST

The following analyses were performed:
1) Effects of L/D condition (C: control, LL: long light, SL: short light) and of vehicle (DMSO, Wat: water):

a) C-Veh, LL-Veh and SL-Veh: analyzed by 2-factor ANOVA (factor 1: L/D condition, factor 2: vehicle).

b) Effect of vehicle (DMSO and Wat) analyzed within each L/D condition by Student's t-test.
2) Effect of L/D condition, 1-factor ANOVA followed if p≦0.10 by

Fisher's PLSD, test performed on:

a) C-Veh, LL-Veh and SL-Veh.

b) Subgroups administered with DMSO IP: C-DMSO, LL-DMSO and SL-DMSO.

c) Subgroups administered with water IP: C-Wat, LL-Wat and SL-Wat.
3) Effects of TCS 1102 and of almorexant:

Because the results showed that in some tests, the behavior was different depending on whether the vehicle was DMSO administered IP or water administered PO, the effect of TCS 1102 was analyzed by comparison of groups administered IP with DMSO and the effect of almorexant was analyzed by comparisons of groups administered PO with water.

1) TCS 1102:
a) 1-factor ANOVA followed if p≦0.10 by Fisher's PLSD, test performed on C-DMSO, LL-DMSO, SL-DMSO and SC-TCS groups.
b) Student's t-test performed on SL-DMSO and SC-TCS groups.

2) Almorexant:
a) 1-factor ANOVA followed if p≦0.10 by Fisher's PLSD, test performed on C-Wat, LL-Wat, SL-Wat and SC-Almo groups.
b) Student's t-test performed on SL-Wat and SC-Almo groups.

A difference was considered:
1) Statistically significant: p≦0.05.
2) Close-to-significant difference: 0.05<p≦0.10.
3) Not significant: p>0.10.

### Marble Burying Test

The same analyses, as described above, were performed, except that C groups and TCS 1102 group were not tested.

### Body Weight

The following analyses were performed:
1) Initial body weight:

Initial body weight (BW) between recorded on day d-4, before the initiation of L/D change analyzed by 1-factor ANOVA and Student's t-test.

These analyses showed close-to-significant differences of BW between groups. Therefore, the following analyses were performed on the BW expressed in percentage of the initial BW.
2) Effect of L/D condition:

The BW expressed in percentage of the initial BW was compared between by ANOVA for repeated measures (from day dl to day d54):
a) On C-Veh, LL-Veh and SL-Veh groups: overall effect.
b) On C-Veh and LL-Veh groups: effect of LL.
c) On C-Veh and SL-Veh groups: effect of SL.
d) On LL-Veh and SL-Veh groups: difference LL vs. SL.
3) Effect of vehicle:

The BW expressed in percentage of the initial BW was compared between by ANOVA for repeated measures (from day d29 to day d54):
a) On C-DMSO, C-Wat, LL-DMSO, LL-Wat, SL-DMSO and SL-Wat groups: effect of vehicle under the three L/D conditions.
b) On C-DMSO and C-Wat groups: effect of vehicle in C condition.
c) On LL-DMSO and LL-Wat groups: effect of vehicle in LL condition.
d) On SL-DMSO and SL-Wat groups: effect of vehicle in SL condition.
4) Effect of TCS 1102 and almorexant:

The BW expressed in percentage of the initial BW was compared between by ANOVA for repeated measures (from day d29 to day d54):
a) Effect of TCS 1102:
   i) On C-DMSO, LL-DMSO, SL-DMSO and SL-TCS groups.
   ii) On SL-DMSO and SL-TCS groups.
b) Effect of almorexant:
   i) On C-Wat, LL-Wat, SL-Wat and SL-Almo groups.
   ii) On SL-Wat and SL-Almo groups.

### Results

Symptoms

Abdominal contractions lasting 10-20 sec were noticed after the IP injections of 20% DMSO and of TCS 1102. These contractions were obviously induced by DMSO.

No other symptom was noticed.

Open-Field Test

First Session

Effects of L/D Condition and of Vehicle

The distance travelled (Distance), the percentage of distance travelled in the center (% Dist Center) and the percentage of time spent in the center (% Time Center) in the C-DMSO, C-Wat, LL-DMSO, LL-Wat, SL-DMSO and SL-Wat groups are presented in Table 3. The Distance, the % Dist Center and the % Time Center in the C-Veh, LL-Veh and SL-Veh groups are presented in Table 4.

The analyses by 2-factor ANOVA of the effects of L/D condition and of vehicle (Table 3) show no significant effect of vehicle and no significant interaction L/D condition×vehicle on the Distance, on the % Dist Center and on the % Time Center.

Comparisons by Student's t-test within each L/D condition of DMSO and Wat treated groups show that the Distance tended to be lower in DMSO group than in Wat group for the LL condition only and that the % Dist Center and the % Time Center tended to be lower in DMSO group than in Wat group for the SL condition only.

CONCLUSION: There was no significant effect of vehicle on the parameters recorded in the open-field.

TABLE 3
Distance travelled in cm, % Dist Center and % Time Center
(mean, SEM) in C-DMSO, C-Wat, LL-DMSO, LL-Wat, SL-DMSO and
SL-Wat groups. Statistical analyses: 2-factor ANOVAs: p
values for the effect of L/D cycle, for the effect of vehicle
and for the interaction L/D cycle × vehicle. Student's
t-test: p values for differences Wat vs. DMSO.

| | | | | % Dist | % Time |
|---|---|---|---|---|---|
| | | | Distance | Cent | Cent |
| C-Veh | DMSO | Mean | 8504.2 | 12.1 | 8.3 |
| | | SEM | 493.5 | 0.7 | 1.1 |
| | Wat | Mean | 8863.1 | 10.8 | 8.6 |
| | | SEM | 323.9 | 2.1 | 2.0 |
| | Student's t-test p ≦ | | 0.6 | 0.6 | 1 |
| LL-Veh | DMSO | Mean | 9055.1 | 10.4 | 7.5 |
| | | SEM | 400.8 | 0.6 | 0.7 |
| | Wat | Mean | 10056.1 | 12.0 | 8.2 |
| | | SEM | 378.4 | 1.5 | 0.7 |
| | Student's t-test p ≦ | | 0.1 | 0.4 | 0.6 |
| SL-Veh | DMSO | Mean | 8712.0 | 5.3 | 4.0 |
| | | SEM | 279.8 | 0.9 | 0.3 |
| | Wat | Mean | 7839.8 | 9.6 | 6.8 |
| | | SEM | 624.5 | 1.8 | 1.4 |
| | Student's t-test p ≦ | | 0.3 | 0.1 | 0.1 |
| 2-Factor | L/D | p ≦ | 0.05 | 0.05 | 0.05 |
| ANOVA | Veh | p ≦ | 0.7 | 0.2 | 0.2 |
| | L/D × Veh | p ≦ | 0.2 | 0.2 | 0.6 |

Effects of L/D Condition

The Distance:

1) Was lower in SL condition than in LL condition; this effect was observed on whole vehicle groups (Table 4), on water treated groups (Table 6), but not on DMSO treated groups (Table 5).

2) Tended to be higher in LL condition than in C condition; this trend was observed on whole vehicle groups (Table 4), on water treated groups (Table 6), but not on DMSO treated groups (Table 5).

3) Was not significantly different between LL condition and C condition (Table 4), (Table 5), (Table 6).

CONCLUSION: The increase in light duration tended to induce an increase of the Distance in the open-field. However, this effect was observed in water treated groups, but not in DMSO treated groups. This effect may be counteracted by the stressful effect of the DMSO injection.

The % Dist Center and the % Time Center:

1) Were lower in SL condition than in both C and LL conditions; these effects were observed on whole vehicle groups (Table 4), on DMSO treated groups (Table 5), but not on water treated groups (Table 6).

2) Were not significantly different between LL condition and C condition (Table 4, Table 5 and Table 6).

CONCLUSION: The decrease in light duration induced a decrease in both the % Dist Center open-field and the % Time Center of the open-field, i.e. induced an anxiogenic-like effect. However, this effect was observed in DMSO treated groups, but not in water treated groups. This effect may be triggered by both the decrease in light duration and the stressful effect of the DMSO injection

TABLE 4
Distance travelled in cm, % Dist Center and % Time Center
(mean, SEM) in C-Veh, LL-Veh and SL-Veh groups. Statistical
analyses: 1-factor ANOVAs: p values for the effect of L/D
cycle and post-hoc comparisons by Fisher's PLSD test.

| | | | % Dist | % Time |
|---|---|---|---|---|
| | | Distance | Cent | Cent |
| C-Veh | Mean | 8683.7 | 11.4 | 8.4 |
| | SEM | 286.6 | 1.1 | 1.1 |
| LL-Veh | Mean | 9555.6 | 11.2 | 7.8 |
| | SEM | 303.0 | 0.8 | 0.5 |
| Fisher's PLSD test | p vs. C-Veh ≦ | 0.1 | 0.9 | 0.7 |
| SL-Veh | Mean | 8275.9 | 7.5 | 5.4 |
| | SEM | 351.7 | 1.1 | 0.8 |
| Fisher's PLSD test | p vs. C-Veh ≦ | 0.4 | 0.01 | 0.05 |
| | p vs. LL-Veh ≦ | 0.01 | 0.05 | 0.05 |
| 1-Factor ANOVA | p ≦ | 0.05 | 0.05 | 0.05 |

TABLE 5
Distance travelled in cm, % Dist Center and % Time Center
(mean, SEM) in C-DMSO, LL-DMSO and SL-DMSO groups. Statistical
analyses: 1-factor ANOVAs: p values for the effect of L/D
cycle and post-hoc comparisons by Fisher's PLSD test.

| | | | % Dist | % Time |
|---|---|---|---|---|
| | | Distance | Cent | Cent |
| C-DMSO | Mean | 8504.2 | 12.1 | 8.3 |
| | SEM | 493.5 | 0.7 | 1.1 |
| LL-DMSO | Mean | 9055.1 | 10.4 | 7.5 |
| | SEM | 400.8 | 0.6 | 0.7 |
| Fisher's PLSD test | p vs. C-DMSO ≦ | NS | 0.2 | 0.5 |
| SL-DMSO | Mean | 8712.0 | 5.3 | 4.0 |
| | SEM | 279.8 | 0.9 | 0.3 |
| Fisher's PLSD test | p vs. C-DMSO ≦ | NS | 0.001 | 0.01 |
| | p vs. LL-DMSO ≦ | NS | 0.001 | 0.01 |
| 1-Factor ANOVA | p ≦ | 0.7 | 0.001 | 0.01 |

TABLE 6
Distance travelled in cm, % Dist Center and % Time Center
(mean, SEM) in C-Wat, LL-Wat and SL-Wat groups. Statistical
analyses: 1-factor ANOVAs: p values for the effect of L/D
cycle and post-hoc comparisons by Fisher's PLSD test.

| | | | % Dist | % Time |
|---|---|---|---|---|
| | | Distance | Cent | Cent |
| C-Wat | Mean | 8863.1 | 10.8 | 8.6 |
| | SEM | 323.9 | 2.1 | 2.0 |
| LL-Wat | Mean | 10056.1 | 12.0 | 8.2 |
| | SEM | 378.4 | 1.5 | 0.7 |
| Fisher's PLSD test | p vs. C-Wat ≦ | 0.1 | NS | NS |
| SL-Wat | Mean | 7839.8 | 9.6 | 6.8 |
| | SEM | 624.5 | 1.8 | 1.4 |
| Fisher's PLSD test | p vs. C-Wat ≦ | 0.2 | NS | NS |
| | p vs. LL-Wat ≦ | 0.01 | NS | NS |
| 1-Factor ANOVA | p ≦ | 0.05 | 0.7 | 0.7 |

Effects of TCS 1102 and of Almorexant

* Effect of TCS 1102: Analyses on C-DMSO, LL-DMSO, SL-DMSO and SL-TCS Groups

ANOVAs show a significant difference between groups only for the % Dist Center but no significant difference between groups for the Distance and the % Time Center (Table 7).

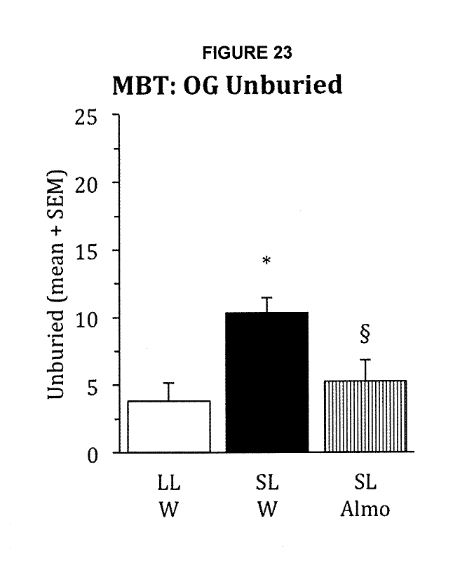

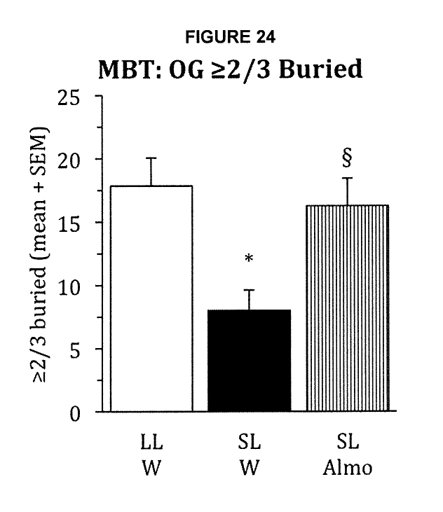

With respect to the figures as referenced hereafter, if not otherwise indicated: p vs LL≦0.05=#, p vs LL≦0.01=##, p vs SL-Veh≦0.05=§, p vs SL-Veh≦0.01=§§ for all non-MBT graphs. For MBT graphs: # is replaced with * in FIGS. 23 and 24.

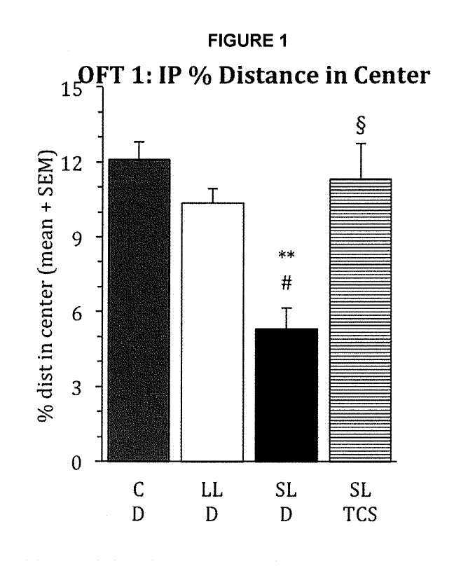

Table 7 and FIG. 1 show that SL condition decreased the % Dist Center in comparison with both C and LL conditions. TCS 1102 reduced the decrease of % Dist Center induced by SL condition. Similar effects were observed on the % Time Center, but they did not reach statistical significance (Table 7). FIG. 1 shows distance travelled as % Distance in Center in C-DMSO, LL-DMSO, SL-DMSO and SL-TCS groups. Results are expressed as mean±SEM. Statistical analyses by Fisher's PLSD tests: difference vs. C-DMSO group ** p≦0.01; difference vs. LL-DMSO group: # p≦0.05; difference vs. SL-DMSO group: §p≦0.05.

Comparisons of SL-DMSO and SL-TCS groups by the Student's t-test show that TCS 1102:
1) Did not significantly modify the Distance (p≦0.8).
2) Increased the % Dist Center (p≦0.05).
3) Tended to increase the % Time Center (0.05<p≦0.10).

CONCLUSION: TCS 1102 reduced the anxiogenic-like effect of short light duration and did not modify the motor exploratory activity.

TABLE 7
Distance travelled in cm, % Dist Center and % Time
Center (mean, SEM) in C-DMSO, LL-DMSO, SL-DMSO and
SL-TCS groups. Statistical analyses: 1-factor ANOVAs:
p values for the effect of L/D cycle and post-hoc
comparisons by Fisher's PLSD test.

| | | | % Dist | % Time |
|---|---|---|---|---|
| | | Distance | Cent | Cent |
| C-DMSO | Mean | 8504.2 | 12.1 | 8.3 |
| | SEM | 493.5 | 0.7 | 1.1 |
| LL-DMSO | Mean | 9055.1 | 10.4 | 7.5 |
| | SEM | 400.8 | 0.6 | 0.7 |
| Fisher's PLSD test | p vs. C-DMSO ≦ | NS | 0.4 | NS |
| SL-DMSO | Mean | 8712.0 | 5.3 | 4.0 |
| | SEM | 279.8 | 0.9 | 0.3 |
| Fisher's PLSD test | p vs. C-DMSO ≦ | NS | 0.01 | NS |
| | p vs. LL-DMSO ≦ | NS | 0.05 | NS |
| SL-TCS | Mean | 8505.8 | 11.3 | 7.4 |
| | SEM | 368.4 | 1.4 | 1.3 |
| Fisher's PLSD test | p vs. C-DMSO ≦ | NS | 0.7 | NS |
| | p vs. LL-DMSO ≦ | NS | 0.6 | NS |
| | p vs. SL-DMSO ≦ | NS | 0.01 | NS |
| 1-Factor ANOVA | p ≦ | 0.8 | 0.01 | 0.2 |

* Effect of Almorexant: Analyses on C-Wat, LL-Wat, SL-Wat and SL-Almo Groups

ANOVAs show a significant difference between groups only for the Distance but no significant difference between groups for the % Dist Center and the % Time Center Table 8.

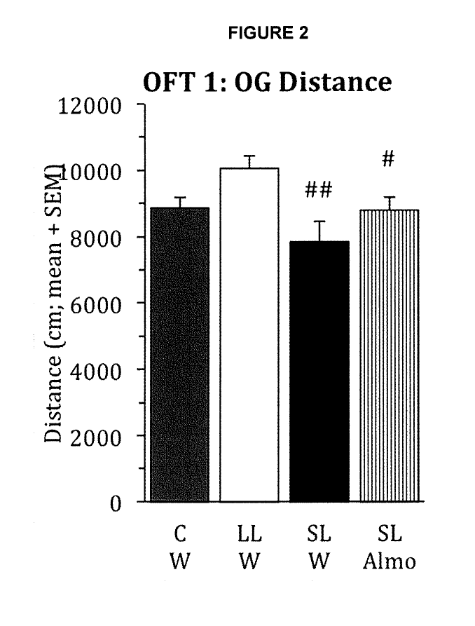

Table 8 and FIG. 2 show that SL condition decreased the Distance in comparison with LL conditions. This effect was not significantly reduced by almorexant. FIG. 2 shows Distance travelled in cm (A) in C-Wat, LL-Wat, SL-Wat and SL-Almo groups. Results are expressed as mean±SEM. Statistical analyses by Fisher's PLSD tests: difference vs. LL-Wat group: # p≦0.05, ## p≦0.01.

Comparisons of SL-Wat and SL-Almo groups by the Student's t-test show that almorexant:
1) Did not significantly modify the Distance (p≦0.2).
2) Did not significantly modify the % Dist Center (p≦0.9).
3) Did not significantly modify the % Time Center (p≦1).

CONCLUSION: Almorexant did not induce effect on anxiety-like behavior and did not modify the motor exploratory activity.

TABLE 8
Distance travelled in cm, % Dist Center and % Time Center (mean,
SEM) in C-Wat, LL-Wat, SL-Wat and SL-Almo groups. Statistical
analyses: 1-factor ANOVAs: p values for the effect of L/D cycle
and post-hoc comparisons by Fisher's PLSD test.

| | | | % Dist | % Time |
|---|---|---|---|---|
| | | Distance | Cent | Cent |
| C-Wat | Mean | 8863.1 | 10.8 | 8.6 |
| | SEM | 323.9 | 2.1 | 2.0 |
| LL-Wat | Mean | 10056.1 | 12.0 | 8.2 |
| | SEM | 378.4 | 1.5 | 0.7 |
| Fisher's PLSD test | p vs. C-Wat ≦ | 0.2 | NS | NS |
| SL-Wat | Mean | 7839.8 | 9.6 | 6.8 |
| | SEM | 624.5 | 1.8 | 1.4 |
| Fisher's PLSD test | p vs. C-Wat ≦ | 0.2 | NS | NS |
| | p vs. LL-Wat ≦ | 0.01 | NS | NS |
| SL-Almo | Mean | 8786.5 | 9.9 | 6.7 |
| | SEM | 386.7 | 0.8 | 0.6 |
| Fisher's PLSD test | p vs. C-Wat ≦ | 1 | NS | NS |
| | p vs. LL-Wat ≦ | 0.05 | NS | NS |
| | p vs. SL-Wat ≦ | 0.2 | NS | NS |
| 1-Factor ANOVA | p ≦ | 0.05 | 0.7 | 0.6 |

Elevated-Plus-Maze

Effects of L/D Condition and of Vehicle

The percentage of time spent in open arms (% TOA), the percentage of open arm entries (% OAE) and the number of arm entries (AE) are presented in Table 9 (C-DMSO, C-Wat, LL-DMSO, LL-Wat, SL-DMSO and SL-Wat groups) and in Table 10 (C-Veh, LL-Veh and SL-Veh groups).

The analyses of the effects of L/D condition and of vehicle by 2-factor ANOVA (Table 9) show:

1) No significant effect of vehicle and no significant interaction L/D condition×vehicle on % TOA, the Distance, on the % Dist Center and on the % Time Center.

2) A close-to-significant effect of vehicle and a close-to-significant interaction L/D condition×vehicle on % OAE.

3) A significant effect of vehicle and significant interaction L/D condition×vehicle on AE.

Comparisons within each L/D condition of DMSO and Wat treated groups show that the % TOA and the % OAE were not significantly different between DMSO group and Wat group in C conditions, were lower in DMSO group than in Wat group in LL condition and tended to be lower in DMSO group than in Wat group in SL condition. The AE was lower in DMSO group than in Wat group in LL condition, but not in C and SL conditions.

CONCLUSION: There was an effect of vehicle on the parameters recorded in the EPM. DMSO injection induced an anxiogenic-like effect that was significant in LL condition, close to significance in SL condition and not observed in C condition. DMSO also decreased activity in SL condition only.

TABLE 9
Percentage of time spent in open arms (% TOA), percentage
of open arm entries (% OAE) and number of arm entries (AE)
(mean, SEM) in C-DMSO, C-Wat, LL-DMSO, LL-Wat, SL-DMSO and
SL-Wat groups. Statistical analyses: 2-factor ANOVAs: p
values for the effect of L/D cycle, for the effect of vehicle
and for the interaction L/D cycle × vehicle. Student's
t-test: p values for differences Wat vs. DMSO.

| | | | % TOA | % OAE | AE |
|---|---|---|---|---|---|
| C-Veh | DMSO | Mean | 36.9 | 39.4 | 19.8 |
| | | SEM | 6.7 | 5.0 | 0.9 |
| | Wat | Mean | 29.6 | 34.6 | 20.5 |
| | | SEM | 6.6 | 4.1 | 1.1 |
| | Student's t-test p ≦ | | 0.5 | 0.5 | 0.7 |
| LL-Veh | DMSO | Mean | 34.2 | 36.4 | 18.8 |
| | | SEM | 3.4 | 3.8 | 0.8 |
| | Wat | Mean | 47.0 | 48.8 | 24.8 |
| | | SEM | 4.3 | 2.3 | 1.2 |
| | Student's t-test p ≦ | | 0.05 | 0.05 | 0.01 |
| SL-Veh | DMSO | Mean | 15.6 | 21.9 | 19.2 |
| | | SEM | 2.6 | 2.5 | 1.2 |
| | Wat | Mean | 26.6 | 31.8 | 20.0 |
| | | SEM | 4.9 | 4.7 | 1.5 |
| | Student's t-test p ≦ | | 0.1 | 0.1 | 0.7 |
| 2-Factor | L/D | p ≦ | 0.01 | 0.01 | 0.2 |
| ANOVA | Veh | p ≦ | 0.2 | 0.1 | 0.01 |
| | L/D × Veh | p ≦ | 0.2 | 0.1 | 0.05 |

Effects of L/D Condition and of Vehicle

The % TOA and the % OAE:
1) Were higher in LL condition than in C condition when they were analyzed on water treated groups (Table 12), but not when they were analyzed on whole vehicle groups (Table 10) and on DMSO treated groups (Table 11).
2) Were lower in SL condition than in both C and LL conditions when they were analyzed on whole vehicle groups (Table 10), and on DMSO treated groups (Table 11) and was lower in SL condition than in LL condition when they were analyzed on water treated groups (Table 12).

The AE:

1) Was not significantly modified when it was analyzed on whole vehicle groups (Table 10) and on DMSO treated groups (Table 11).

2) Was higher in LL condition than in both C and SL conditions when it was analyzed on water treated groups (Table 12).

CONCLUSION: The increase in light duration increased the % TOA, the % OAE and the AE, i.e. induced an anxiolytic-like effect and a disinhibitory effect, in water treated group, but not in DMSO treated group.

CONCLUSION: The decrease in light duration decreased the % TOA and the % OAE, i.e. induced an anxiogenic-like effect, in DMSO treated group, but not in water treated group.

TABLE 10
Percentage of time spent in open arms (% TOA), percentage
of open arm entries (% OAE) and number of arm entries (AE)
(mean, SEM) in C-Veh, LL-Veh and SL-Veh groups. Statistical
analyses: 1-factor ANOVAs: p values for the effect of L/D
cycle and post-hoc comparisons by Fisher's PLSD test.

| | | % TOA | % OAE | AE |
|---|---|---|---|---|
| C-Veh | Mean | 33.3 | 37.0 | 20.2 |
| | SEM | 4.6 | 3.2 | 0.7 |
| LL-Veh | Mean | 40.6 | 42.6 | 21.8 |
| | SEM | 3.2 | 2.8 | 1.1 |
| Fisher's PLSD test | p vs. C-Veh ≦ | 0.2 | 0.2 | NS |
| SL-Veh | Mean | 21.1 | 26.9 | 19.6 |
| | SEM | 3.1 | 2.9 | 0.9 |
| Fisher's PLSD test | p vs. C-Veh ≦ | 0.05 | 0.05 | NS |
| | p vs. LL-Veh ≦ | 0.001 | 0.001 | NS |
| 1-Factor ANOVA | p ≦ | 0.01 | 0.01 | 0.3 |

TABLE 11
Percentage of time spent in open arms (% TOA), percentage of
open arm entries (% OAE) and number of arm entries (AE)
(mean, SEM) in C-DMSO, LL-DMSO and SL-DMSO groups.
Statistical analyses: 1-factor ANOVAs: p values for the effect
of L/D cycle and post-hoc comparisons by Fisher's PLSD test.

| | | % TOA | % OAE | AE |
|---|---|---|---|---|
| C-DMSO | Mean | 36.9 | 39.4 | 19.8 |
| | SEM | 6.7 | 5.0 | 0.9 |
| LL-DMSO | Mean | 34.2 | 36.4 | 18.8 |
| | SEM | 3.4 | 3.8 | 0.8 |
| Fisher's PLSD test | p vs. C-DMSO ≦ | 0.7 | 0.6 | NS |
| SL-DMSO | Mean | 15.6 | 21.9 | 19.2 |
| | SEM | 2.6 | 2.5 | 1.2 |
| Fisher's PLSD test | p vs. C-DMSO ≦ | 0.01 | 0.01 | NS |
| | p vs. LL-DMSO ≦ | 0.05 | 0.05 | NS |
| 1-Factor ANOVA | p ≦ | 0.01 | 0.05 | 0.8 |

TABLE 12
Percentage of time spent in open arms (% TOA), percentage of
open arm entries (% OAE) and number of arm entries (AE)
(mean, SEM) in C-Wat, LL-Wat and SL-Wat groups. Statistical
analyses: 1-factor ANOVAs: p values for the effect of L/D cycle
and post-hoc comparisons by Fisher's PLSD test.

| | | % TOA | % OAE | AE |
|---|---|---|---|---|
| C-Wat | Mean | 29.6 | 34.6 | 20.5 |
| | SEM | 6.6 | 4.1 | 1.1 |
| LL-Wat | Mean | 47.0 | 48.8 | 24.8 |
| | SEM | 4.3 | 2.3 | 1.2 |
| Fisher's PLSD test | p vs. C-Wat ≦ | 0.05 | 0.05 | 0.05 |
| SL-Wat | Mean | 26.6 | 31.8 | 20.0 |
| | SEM | 4.9 | 4.7 | 1.5 |
| Fisher's PLSD test | p vs. C-Wat ≦ | 0.7 | 0.7 | 0.8 |
| | p vs. LL-Wat ≦ | 0.05 | 0.01 | 0.05 |
| 1-Factor ANOVA | p ≦ | 0.05 | 0.05 | 0.05 |

Effects of TCS 1102 and of Almorexant

* Effect of TCS 1102: Analyses on C-DMSO, LL-DMSO, SL-DMSO and SL-TCS Groups

ANOVAs show a significant difference between groups only for the % TOA and the % OAE, but no significant difference between groups for the AE (Table 13).

Table 13 show that TCS 1102:
1) Did not significantly reduce the decrease of % TOA induced by SL condition).
2) Tended to reduce the decrease of % OAE induced by SL condition.

Comparisons of SL-DMSO and SL-TCS groups by the Student's t-test show that TCS 1102:

1) Did not significantly modify the % TOA (p≦0.2).

2) Increased the % OAE (p≦0.05).

3) Did not significantly modify the AE (p≦0.4).

CONCLUSION: TCS 1102 tended to reduce the anxiogenic-like effect of short light duration.

TABLE 13
Percentage of time spent in open arms (% TOA), percentage of
open arm entries (% OAE) and number of arm entries (AE)
(mean, SEM) in C-DMSO, LL-DMSO, SL-DMSO and SL-TCS
groups. Statistical analyses: 1-factor ANOVAs: p values for the
effect of L/D cycle and post-hoc comparisons by Fisher's
PLSD test.

| | | % TOA | % OAE | AE |
|---|---|---|---|---|
| C-DMSO | Mean | 36.9 | 39.4 | 19.8 |
| | SEM | 6.7 | 5.0 | 0.9 |
| LL-DMSO | Mean | 34.2 | 36.4 | 18.8 |
| | SEM | 3.4 | 3.8 | 0.8 |
| Fisher's PLSD test | p vs. C-DMSO ≦ | 0.7 | 0.6 | NS |
| SL-DMSO | Mean | 15.6 | 21.9 | 19.2 |
| | SEM | 2.6 | 2.5 | 1.2 |
| Fisher's PLSD test | p vs. C-DMSO ≦ | 0.01 | 0.01 | NS |
| | p vs. LL-DMSO ≦ | 0.01 | 0.05 | NS |
| SL-TCS | Mean | 23.1 | 32.0 | 16.8 |
| | SEM | 3.0 | 2.9 | 1.5 |
| Fisher's PLSD test | p vs. C-DMSO ≦ | 0.05 | 0.2 | NS |
| | p vs. LL-DMSO ≦ | 0.1 | 0.4 | NS |
| | p vs. SL-DMSO ≦ | 0.2 | 0.1 | NS |
| 1-Factor ANOVA | p ≦ | 0.01 | 0.05 | 0.5 |

Effect of almorexant: analyses on C-Wat, LL-Wat, SL-Wat and SL-Almo groups

ANOVAs show a significant difference between groups for the % TOA, the % OAE and the AE (Table 14).

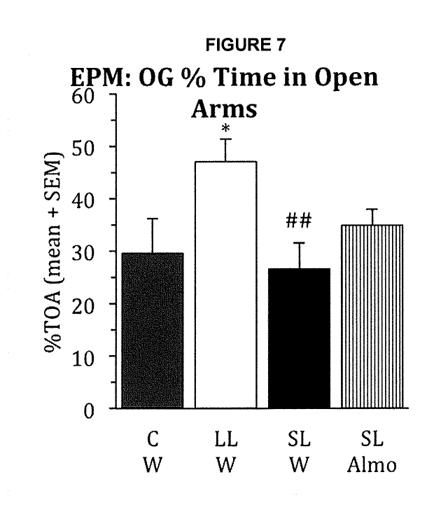

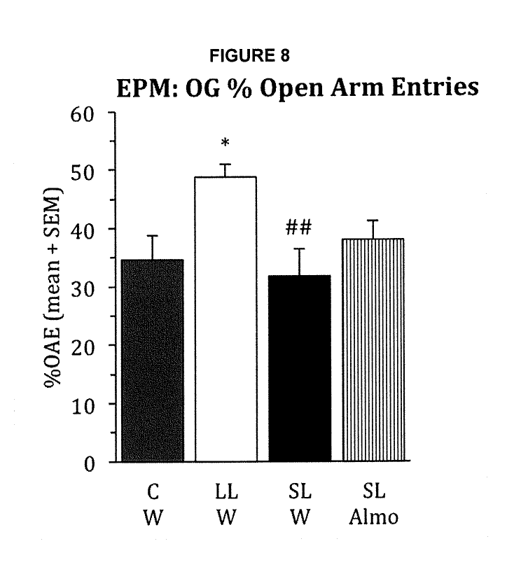

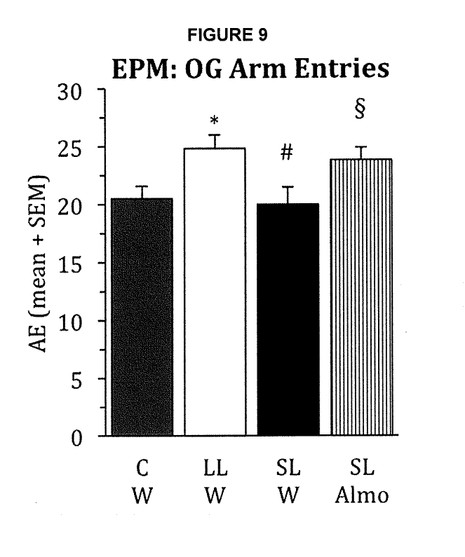

Table 14 show that almorexant had no significant effect on the % TOA (FIG. 7) and on the % OAE (FIG. 8) but increased the AE (FIG. 9).

Comparisons of SL-Wat and SL-Almo groups by the Student's t-test show that almorexant:

1) Did not significantly modify the % TOA (p≦0.2).

2) Did not significantly modify the % OAE (p≦0.3).

3) Tended to increase the AE (p≦0.10).

CONCLUSION: Almorexant did not induce effect on anxiety-like behavior and increased the motor exploratory activity.

TABLE 14
Percentage of time spent in open arms (% TOA), percentage of
open arm entries (% OAE) and number of arm entries (AE)
(mean, SEM) in C-Wat, LL-Wat, SL-Wat and SL-Almo groups.
Statistical analyses: 1-factor ANOVAs: p values for the effect
of L/D cycle and post-hoc comparisons by Fisher's PLSD test.

| | | % TOA | % OAE | AE |
|---|---|---|---|---|
| C-Wat | Mean | 29.6 | 34.6 | 20.5 |
| | SEM | 6.6 | 4.1 | 1.1 |
| LL-Wat | Mean | 47.0 | 48.8 | 24.8 |
| | SEM | 4.3 | 2.3 | 1.2 |
| Fisher's PLSD test | p vs. C-Wat ≦ | 0.05 | 0.05 | 0.05 |
| SL-Wat | Mean | 26.6 | 31.8 | 20.0 |
| | SEM | 4.9 | 4.7 | 1.5 |
| Fisher's PLSD test | p vs. C-Wat ≦ | 0.7 | 0.7 | 0.9 |
| | p vs. LL-Wat ≦ | 0.01 | 0.01 | 0.05 |
| SL-Almo | Mean | 34.9 | 38.1 | 23.8 |
| | SEM | 3.0 | 3.2 | 1.1 |
| Fisher's PLSD test | p vs. C-Wat ≦ | 0.4 | 0.6 | 0.1 |
| | p vs. LL-Wat ≦ | 0.1 | 0.05 | 0.6 |
| | p vs. SL-Wat ≦ | 0.2 | 0.3 | 0.05 |
| 1-Factor ANOVA | p ≦ | 0.05 | 0.05 | 0.05 |

FIG. 7 shows the percentage of time spent in open arms (% TOA). FIG. 8 shows the percentage of open arm entries (% OAE). FIG. 9 shows the number of arm entries (AE). All of FIGS. 7, 8 and 9 are for C-Wat, LL-Wat, SL-Wat and SL-Almo groups. For FIGS. 7, 8 and 9 the results are expressed as mean±SEM. Statistical analyses by Fisher's PLSD tests: difference vs. C-Wat group: * p≦0.05; difference vs. LL-Wat group: # p≦0.05, ## p≦0.01; difference vs. SL-Wat group: §p.≦0.05.

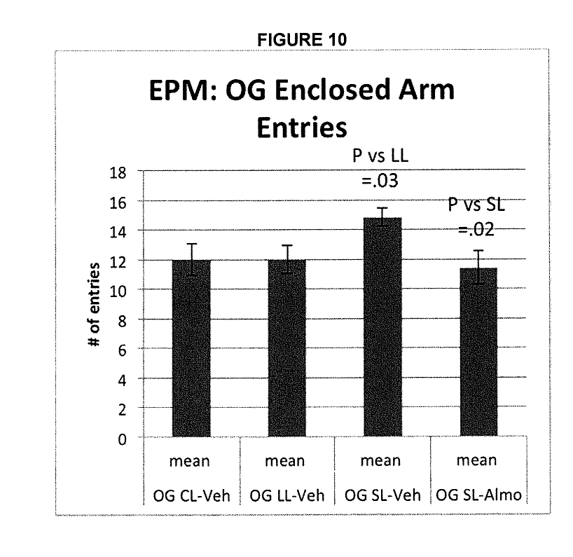

FIG. 10 shows an increase in anxiety-like behaviors in the EPM. Rats exposed to a SL photoperiod increased time spent in the enclosed arms of EPM, which demonstrates increased anxiety and general withdrawal.

### Forced Swimming Test

Second Session

Effects of L/D Condition and of Vehicle

The immobility time (Imo), the swimming time (Swm) and the climbing time (Clb) in the C-DMSO, C-Wat, LL-DMSO, LL-Wat, SL-DMSO and SL-Wat groups are presented in Table 15. The immobility time, the swimming time and the climbing time in the C-Veh, LL-Veh and SL-Veh groups are presented in Table 16.

The analyses by 2-factor ANOVA of the effects of L/D condition and of vehicle show no significant effect of vehicle and no significant interaction L/D condition×vehicle on Imo, Swm and Clb.

Comparisons by Student's t-test within each L/D condition of DMSO and Wat treated groups show a close-to-significant increase of Imo in DMSO group vs. Wat group in C condition only and no significant effect of vehicle on Swm and on Clb.

CONCLUSION: There was no significant effect of vehicle on the parameters recorded in FST.

TABLE 15
Immobility time (Imo), swimming time (Swim) and climbing
time (Clb) (in s; mean, SEM) in C-DMSO, C-Wat, LL-DMSO,
LL-Wat, SL-DMSO and SL-Wat groups. Statistical analyses:
2-factor ANOVAs: p values for the effect of L/D cycle, for
the effect of vehicle and for the interaction L/D cycle × vehicle.
Student's t-test: p values for differences Wat vs. DMSO.

| | | | Imo | Swm | Clb |
|---|---|---|---|---|---|
| C-Veh | DMSO | Mean | 124.8 | 116.0 | 59.2 |
| | | SEM | 17.3 | 9.2 | 21.7 |
| | Wat | Mean | 179.4 | 93.0 | 27.7 |
| | | SEM | 21.8 | 17.4 | 5.9 |
| | Student's t-test p ≦ | | 0.1 | 0.3 | 0.2 |
| LL-Veh | DMSO | Mean | 93.4 | 123.3 | 83.4 |
| | | SEM | 22.0 | 21.2 | 13.9 |
| | Wat | Mean | 112.1 | 89.6 | 98.3 |
| | | SEM | 14.3 | 11.2 | 21.9 |
| | Student's t-test p ≦ | | 0.5 | 0.2 | 0.6 |
| SL-Veh | DMSO | Mean | 154.2 | 114.4 | 31.4 |
| | | SEM | 25.6 | 19.6 | 15.2 |
| | Wat | Mean | 156.4 | 110.1 | 33.6 |
| | | SEM | 13.4 | 16.0 | 6.3 |
| | Student's t-test p ≦ | | 1 | 0.9 | 0.9 |
| 2-Factor | L/D | p ≦ | 0.05 | 0.9 | 0.01 |
| ANOVA | Veh | p ≦ | 0.2 | 0.2 | 0.7 |
| | L/D × Veh | p ≦ | 0.5 | 0.7 | 0.4 |

Effects of L/D Condition

Comparisons by 1-factor ANOVAs show that:

1) Imo was significantly lower in LL condition than in both C condition and SL condition. This effect was significant on all the Veh groups (Table 16) and on Wat-treated groups (Table 18), but not on DMSO-treated groups (Table 17).

2) Swm was not significantly different between the three L/D conditions (Table 16, Table 17, Table 18).

3) Clb was significantly higher in LL condition than in both C condition and SL condition. This effect was significant on all the Veh groups (Table 16) and on Wat-treated groups (Table 18), but not on DMSO-treated groups (Table 17).

CONCLUSION: The SL condition did not significantly alter the behavior in the FST, but the LL condition induced an antidepressant-like effect.

TABLE 16
Immobility time (Imo), swimming time (Swim) and climbing time
(Clb) (mean, SEM) in C-Veh, LL-Veh and SL-Veh groups.
Statistical analyses: 1-factor ANOVAs: p values for the effect of
L/D cycle and post-hoc comparisons by Fisher's PLSD test.

| | | Imo | Swm | Clb |
|---|---|---|---|---|
| C-Veh | Mean | 152.1 | 104.5 | 43.4 |
| | SEM | 15.6 | 10.0 | 11.7 |
| LL-Veh | Mean | 102.7 | 106.4 | 90.8 |
| | SEM | 12.8 | 12.5 | 12.5 |
| Fisher's PLSD test | p vs. C-Veh ≦ | 0.05 | NS | 0.01 |
| SL-Veh | Mean | 155.3 | 112.3 | 32.5 |
| | SEM | 13.8 | 12.1 | 7.9 |
| Fisher's PLSD test | p vs. C-Veh ≦ | 0.9 | NS | 0.5 |
| | p vs. LL-Veh ≦ | 0.05 | NS | 0.001 |
| 1-Factor ANOVA | p ≦ | 0.05 | 0.9 | 0.01 |

TABLE 17
Immobility time (Imo), swimming time (Swim) and climbing time
(Clb) (mean, SEM) in C-DMSO, LL-DMSO and SL-DMSO
groups. Statistical analyses: 1-factor ANOVAs: p values for the
effect of L/D cycle and post-hoc comparisons by Fisher's
PLSD test.

| | | Imo | Swm | Clb |
|---|---|---|---|---|
| C-DMSO | Mean | 124.8 | 116.0 | 59.2 |
| | SEM | 17.3 | 9.2 | 21.7 |
| LL-DMSO | Mean | 93.4 | 123.3 | 83.4 |
| | SEM | 22.0 | 21.2 | 13.9 |
| Fisher's PLSD test | p vs. C-DMSO ≦ | NS | NS | NS |
| SL-DMSO | Mean | 154.2 | 114.4 | 31.4 |
| | SEM | 25.6 | 19.6 | 15.2 |
| Fisher's PLSD test | p vs. C-DMSO ≦ | NS | NS | NS |
| | p vs. LL-DMSO ≦ | NS | NS | NS |
| 1-Factor ANOVA | p ≦ | 0.2 | 1 | 0.2 |

TABLE 18
Immobility time (Imo), swimming time (Swim) and climbing time
(Clb) (mean, SEM) in C-Wat, LL-Wat and SL-Wat groups.
Statistical analyses: 1-factor ANOVAs: p values for the effect of
L/D cycle and post-hoc comparisons by Fisher's PLSD test.

| | | Imo | Swm | Clb |
|---|---|---|---|---|
| C-Wat | Mean | 179.4 | 93.0 | 27.7 |
| | SEM | 21.8 | 17.4 | 5.9 |
| LL-Wat | Mean | 112.1 | 89.6 | 98.3 |
| | SEM | 14.3 | 11.2 | 21.9 |
| Fisher's PLSD test | p vs. C-Wat ≦ | 0.05 | NS | 0.01 |
| SL-Wat | Mean | 156.4 | 110.1 | 33.6 |
| | SEM | 13.4 | 16.0 | 6.3 |
| Fisher's PLSD test | p vs. C-Wat ≦ | 0.4 | NS | 0.8 |
| | p vs. LL-Wat ≦ | 0.1 | NS | 0.01 |
| 1-Factor ANOVA | p ≦ | 0.05 | 0.6 | 0.01 |

Effects of Almorexant

Effect of almorexant: analyses on C-Wat, LL-Wat, SL-Wat and SL-Almo groups

ANOVAs show a significant difference between groups for the Imo and the Clb, and no significant difference between groups for the Swm (Table 19).

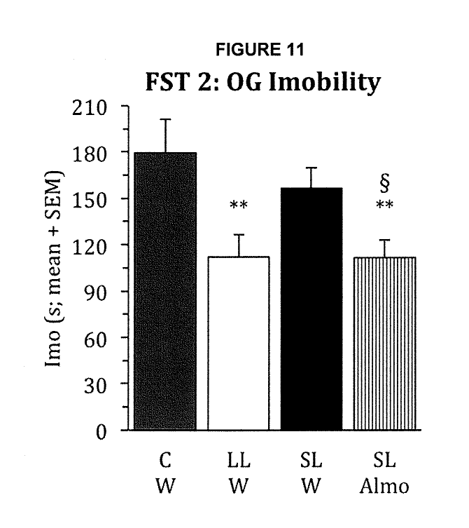

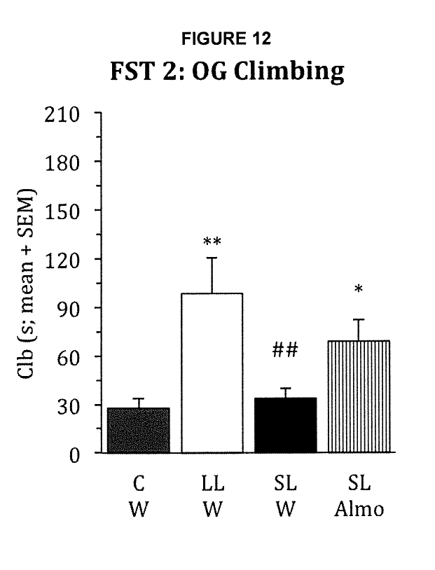

Table 19 show that almorexant decreased Imo (FIG. 11, significant differences vs. C-Wat and SL-Wat groups) and increased Clb (FIG. 12, significant difference vs. C-Wat group and close-to-significant difference vs. SL-Wat group).

Comparisons of SL-Wat and SL-Almo groups by the Student's t-test confirmed that almorexant:

1) Decreased the Imo (p≦0.05).

2) Did not significantly modify the Swm (p≦0.6).

3) Tended to increase the Clb (p≦0.1).

CONCLUSION: Almorexant induced an antidepressant-like effect.

TABLE 19
Immobility time (Imo), percentage swimming time (Swim) and
climbing time (Clb) (mean, SEM) in C-Wat, LL-Wat, SL-Wat
and SL-Almo groups. Statistical analyses: 1-factor ANOVAs:
p values for the effect of L/D cycle and post-hoc comparisons
by Fisher's PLSD test.

| | | Imo | Swm | Clb |
|---|---|---|---|---|
| C-Wat | Mean | 179.4 | 93.0 | 27.7 |
| | SEM | 21.8 | 17.4 | 5.9 |
| LL-Wat | Mean | 112.1 | 89.6 | 98.3 |
| | SEM | 14.3 | 11.2 | 21.9 |
| Fisher's PLSD test | p vs. C-Wat ≦ | 0.01 | NS | 0.01 |
| SL-Wat | Mean | 156.4 | 110.1 | 33.6 |
| | SEM | 13.4 | 16.0 | 6.3 |
| Fisher's PLSD test | p vs. C-Wat ≦ | 0.4 | NS | 0.8 |
| | p vs. LL-Wat ≦ | 0.1 | NS | 0.01 |
| SL-Almo | Mean | 111.5 | 119.8 | 68.7 |
| | SEM | 11.4 | 6.4 | 13.2 |
| Fisher's PLSD test | p vs. C-Wat ≦ | 0.01 | NS | 0.05 |
| | p vs. LL-Wat ≦ | 1 | NS | 0.2 |
| | p vs. SL-Wat ≦ | 0.05 | NS | 0.1 |
| 1-Factor ANOVA | p ≦ | 0.01 | 0.2 | 0.05 |

FIG. 11 shows Immobility time (Imo) and FIG. 12 shows climbing time (Clb) (C), both figures for C-Wat, LL-Wat, SL-Wat and SL-Almo groups. For both figures the results are expressed as mean±SEM. Statistical analyses by Fisher's PLSD tests: difference vs. LL-Wat group: # p≦0.05, ## p≦0.01.

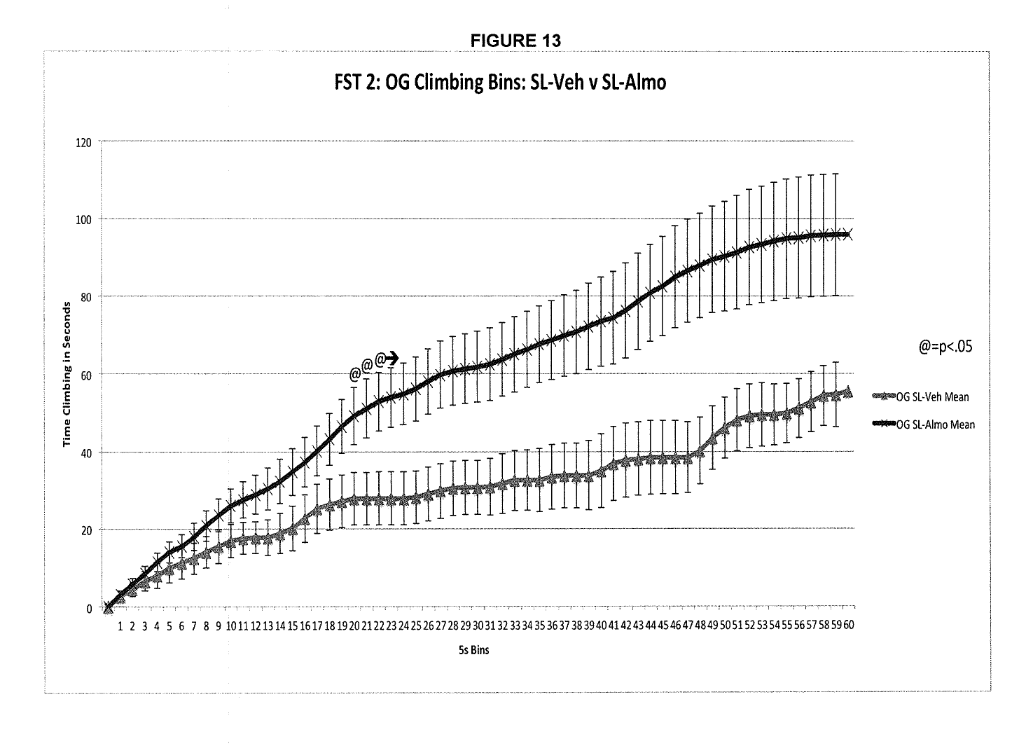

FIG. 13 shows FST 2 binned results for climbing time for both SL-Veh mean and SL-Almo mean.

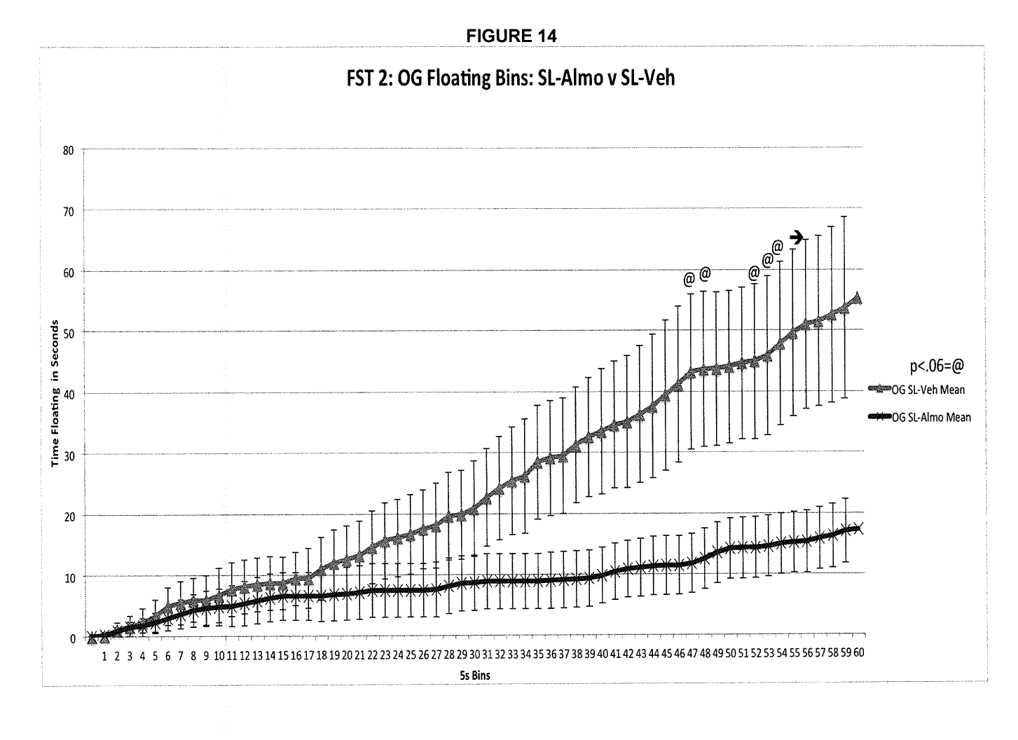

FIG. 14 shows FST 2 binned results for floating time for both SL-Veh mean and SL-Almo mean.

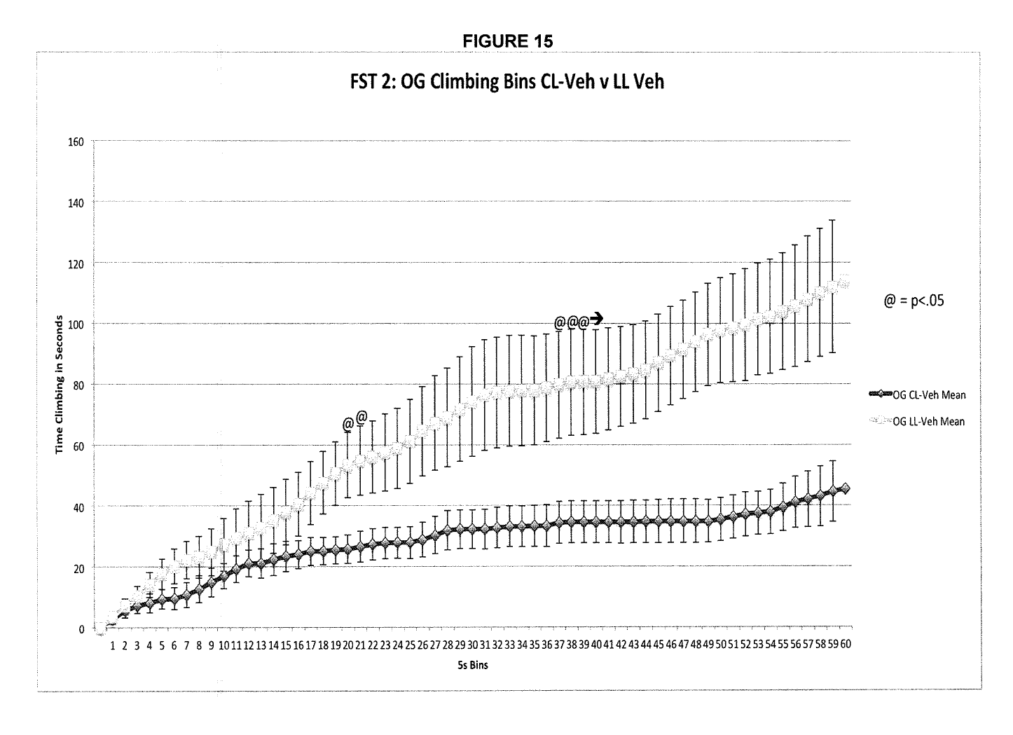

FIG. 15 shows FST 2 binned results for climbing time for both CL (control light)-Veh mean and LL-Veh mean.

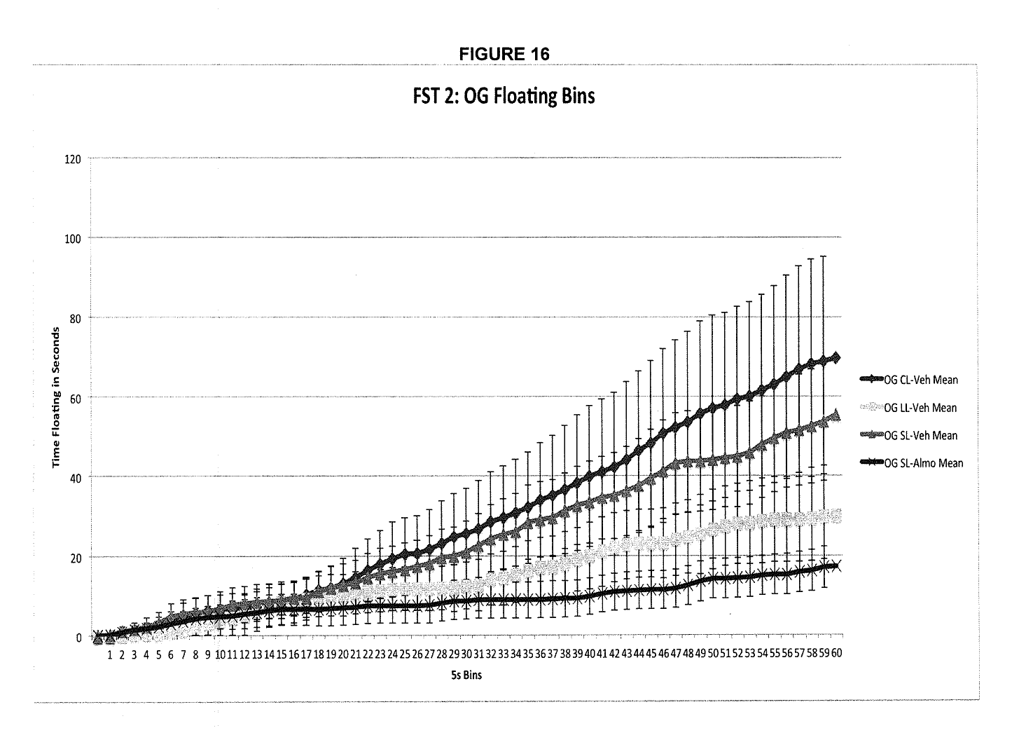

FIG. 16 shows FST 2 binned results for floating time for CL-Veh mean, LL-Veh mean, SL-Veh mean and SL-Almo mean.

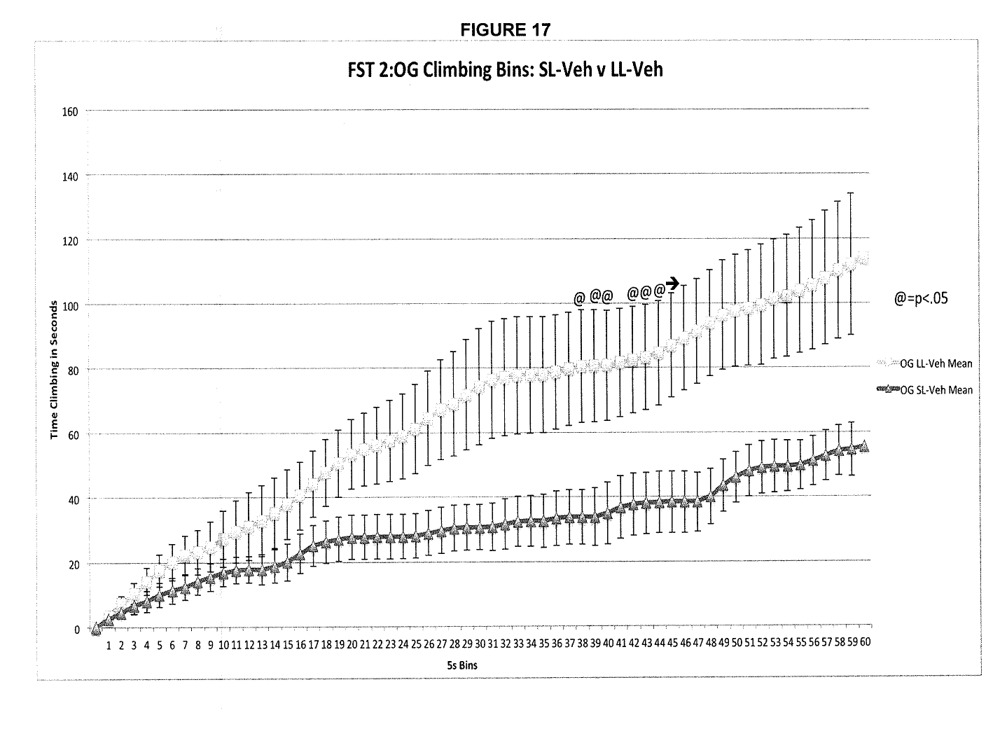

FIG. 17 shows FST 2 binned results for climbing time for both SL-Veh mean and LL-Veh mean.

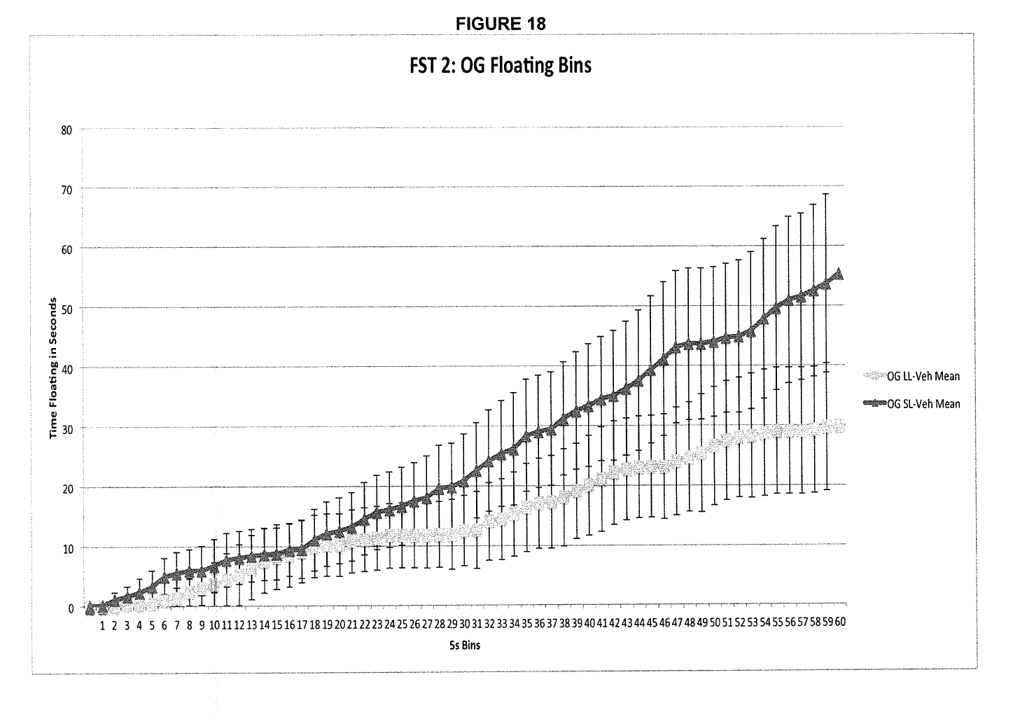

FIG. 18 shows FST 2 binned results for floating time for both LL-Veh mean and SL-Veh mean.

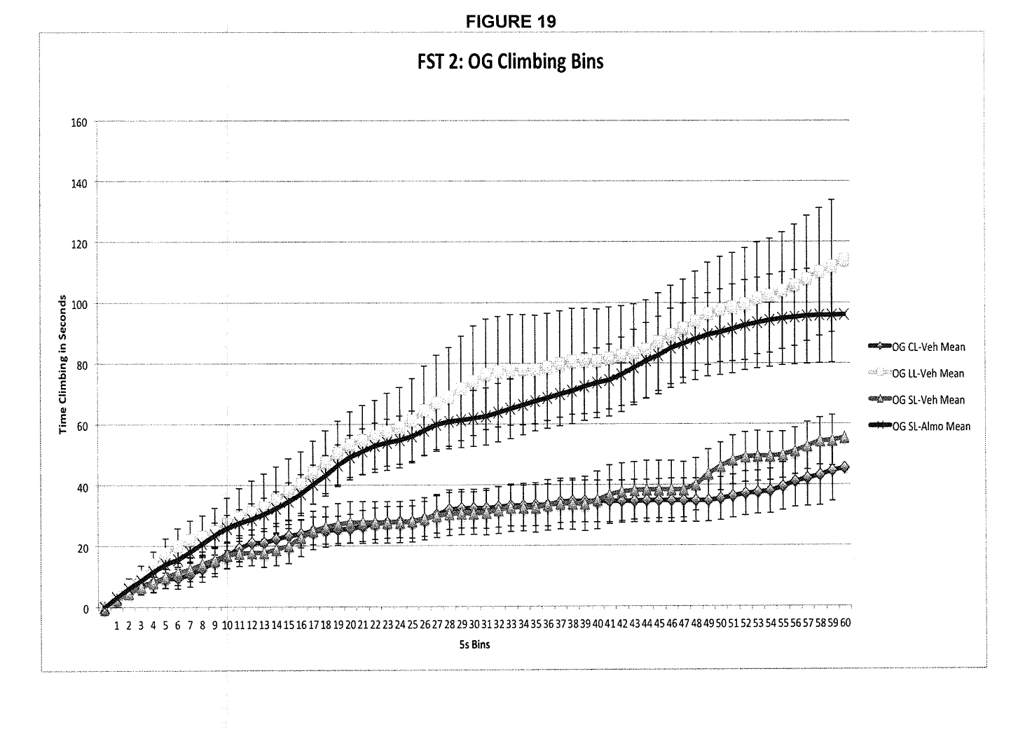

FIG. 19 shows FST 2 binned results for climbing time for CL-Veh mean, LL-Veh mean, SL-Veh mean and SL-Almo mean.

Effect of Experimenter

There was no significant effect of the experimenter on the Imo, but a significant effect of experimenter (p≦0.01) and a significant interaction experimenter×group (p≦0.05) on the Swm and a significant interaction experimenter×group (p≦0.05) on the Clb.

### Marble Burying Test

Effects of L/D Condition and of Vehicle

The number of unburied marbles, the number of ⅔ buried marbles and the number of totally buried marbles are presented in Table 20 (LL-DMSO, LL-Wat, SL-DMSO and SL-Wat groups) and in Table 21 (LL-Veh and SL-Veh groups).

The analyses of the effects of L/D condition and of vehicle by 2-factor ANOVA (Table 9) show:

1) A significant effect of vehicle on the three parameters.

2) A significant interaction L/D condition×vehicle on ⅔ buried and Tot buried.

Comparisons within each L/D condition of DMSO and Wat treated groups show that:

3) In LL condition:

a) Unburied was higher in DMSO group than in Wat group.

b) ≧⅔ buried and Tot buried were lower in DMSO group than in Wat group.

4) In SL condition: Unburied, ≧⅔ buried and Tot buried were not significantly different between DMSO group and Wat group.

CONCLUSION: There was an effect of vehicle on the parameters recorded in the Marble burying test.

TABLE 20
Number of unburied marbles, number of ≧⅔ buried marbles
and number of totally buried (mean, SEM) in LL-DMSO,
LL-Wat, SL-DMSO and SL-Wat groups. Statistical analyses:
2-factor ANOVAs: p values for the effect of L/D cycle, for
the effect of vehicle and for the interaction L/D cycle × vehicle.
Student's t-test: p values for differences Wat vs. DMSO.

| | | | Unburied | ≧⅔ buried | Tot buried |
|---|---|---|---|---|---|
| LL-Veh | DMSO | Mean | 10.8 | 6.7 | 0.5 |
| | | SEM | 1.6 | 1.5 | 0.3 |
| | Wat | Mean | 3.8 | 17.8 | 10.2 |
| | | SEM | 1.4 | 2.2 | 3.2 |
| | Student's t-test p ≦ | | 0.01 | 0.01 | 0.05 |
| SL-Veh | DMSO | Mean | 12.2 | 6.3 | 1.7 |
| | | SEM | 1.9 | 2.0 | 1.3 |
| | Wat | Mean | 10.3 | 8.0 | 3.3 |
| | | SEM | 1.1 | 1.6 | 1.5 |
| | Student's t-test p ≦ | | 0.5 | 0.6 | 0.5 |
| 2-Factor | L/D | p ≦ | 0.05 | 0.05 | 0.2 |
| ANOVA | Veh | p ≦ | 0.01 | 0.01 | 0.01 |
| | L/D × Veh | p ≦ | 0.2 | 0.05 | 0.05 |

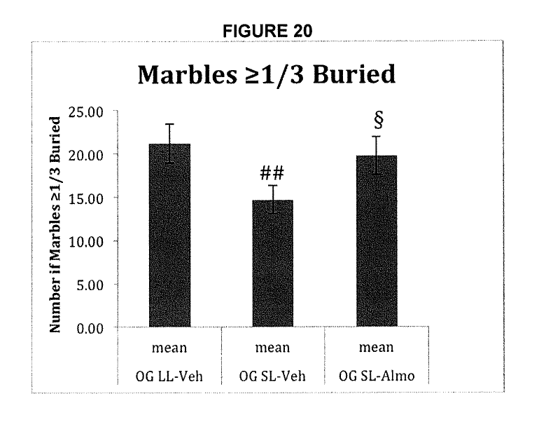

FIG. 20 shows that when a marble is buried in the MBT greater than ⅓, it means that the rat spent some time trying to rid their environment of a slight stressor. A decreased want or ability to get rid of the slight stressors in the SL demonstrates an inability to work efficiently. This may model human seasonal withdrawal in Bipolar patients and SAD patients who withdraw, experience mood disturbances, and work inefficiently in the fall/winter. In the spring/summer, these patients are more able to deal with slight stressors in their environments, are more active, and work more efficiently. A strong therapeutic effect here is shown with Almorexant treatment of the SL rats who bury significantly fewer marbles greater than ⅓.

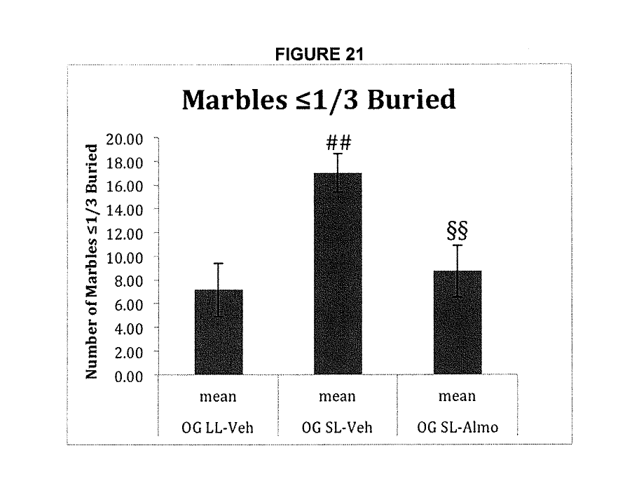

FIG. 21 shows that if a marble in the MBT is left roughly ⅓ or less buried, it means that little was done to remove the slight stressor from the environment. Again, this demonstrates behaviors translational to human SAD and Bipolar withdrawal, inefficiency, inactivity, and accompanying mood disturbances in the fall/winter. With Almorexant treatment, these behaviors were significantly reduced, indicating the potential effectiveness of DORAs in the treatment of seasonal Bipolar symptoms and SAD in humans.

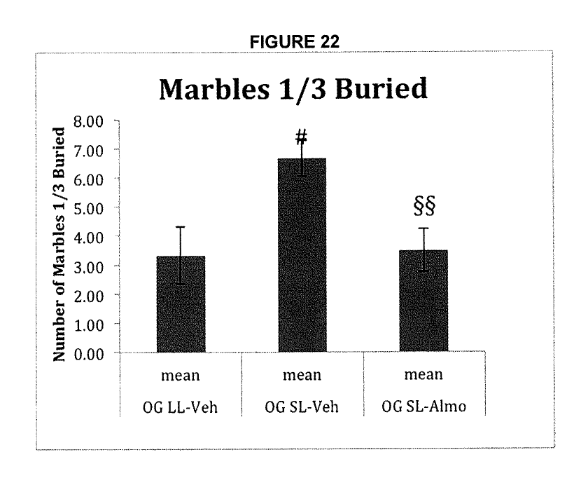

FIG. 22 shows (similar to FIG. 21 where marbles less than or equal to ⅓ are buried) how many marbles were buried roughly ⅓ of the way. Normal behavior, as evidenced by the LL photoperiod, is for few marbles to only be buried ⅓ of the way and for marbles to generally be buried more than ⅓. Completely, and more than ⅓ buried marble display a better ability to rid an environment of slight stressor, to work efficiently, and to interact more with the environment. It is shown here that that these beneficial behaviors are decreased in the SL, and treated statistically significantly with Almorexant.

Effects of L/D Condition and of Vehicle

The Unburied was higher in SL condition than in LL condition. This difference was significant when it was analyzed on whole vehicle groups (Table 21) and on water treated groups (Table 23), but not when it was analyzed on DMSO treated groups (Table 22).

The ≧⅔ buried was lower in SL condition than in LL condition. This difference was close to significance when it was analyzed on whole vehicle groups (Table 21), was significant when it was analyzed on water treated groups (Table 23), but was not observed when it was analyzed on DMSO treated groups (Table 22).

The Tot buried tended to be lower in SL condition than in LL condition when it was analyzed on water treated groups (Table 23), but not when it was analyzed on whole vehicle groups (Table 21), and on DMSO treated groups (Table 22).

CONCLUSION: The decrease in light duration induced a decrease in the number of marbles buried, in comparison with the long light condition. This effect may reveal either an anxiolytic effect or a decrease of activity.

TABLE 21
Number of unburied marbles, number of ≧⅔ buried marbles
and number of totally buried (mean, SEM) in LL-Veh and
SL-Veh groups. Statistical analyses: 1-factor ANOVAs:
p values for the effect of L/D cycle.

| | | Unburied | ≧⅔ buried | Tot buried |
|---|---|---|---|---|
| LL-Veh | Mean | 7.3 | 12.3 | 5.3 |
| | SEM | 1.5 | 2.1 | 2.1 |
| SL-Veh | Mean | 11.3 | 7.2 | 2.5 |
| | SEM | 1.1 | 1.2 | 1.0 |
| Student's t-test p ≦ | p ≦ | 0.05 | 0.1 | 0.3 |

TABLE 22
Number of unburied marbles, number of ≧⅔ buried marbles
and number of totally buried (mean, SEM) in LL-DMSO and
SL-DMSO groups. Statistical analyses: 1-factor ANOVAs:
p values for the effect of L/D cycle.

| | | Unburied | ≧⅔ buried | Tot buried |
|---|---|---|---|---|
| LL-DMSO | Mean | 10.8 | 6.7 | 0.5 |
| | SEM | 1.6 | 1.5 | 0.3 |
| SL-DMSO | Mean | 12.2 | 6.3 | 1.7 |
| | SEM | 1.9 | 2.0 | 1.3 |
| Student's t-test p ≦ | p ≦ | 0.7 | 0.9 | 0.4 |

TABLE 23
Number of unburied marbles, number of ≧⅔ buried marbles
and number of totally buried (mean, SEM) in LL-Wat and
SL-Wat groups. Statistical analyses: 1-factor ANOVAs:
p values for the effect of L/D cycle.

| | | Unburied | ≧⅔ buried | Tot buried |
|---|---|---|---|---|
| LL-Wat | Mean | 3.8 | 17.8 | 10.2 |
| | SEM | 1.4 | 2.2 | 3.2 |
| SL-Wat | Mean | 10.3 | 8.0 | 3.3 |
| | SEM | 1.1 | 1.6 | 1.5 |
| Student's t-test p ≦ | p ≦ | 0.01 | 0.01 | 0.1 |

Effects of Almorexant

Analyses were performed on LL-Wat, SL-Wat and SL-Almo groups ANOVAs show a significant difference between groups for the numbers of unburied and of ≧⅔ buried marbles, but not on the number of Tot buried (Table 24).

Table 24 and comparisons of SL-Wat and SL-Almo groups by the Student's t-test show that Almorexant:

1) Reduced the increase of unburied marbles induced by SL (FIG. 23).

2) Reduced the decrease of ≧⅔ buried marbles induced by SL (FIG. 24).

CONCLUSION: The decrease in the number of marbles buried induced by short light duration, was reduced by Almorexant.

TABLE 24
Number of unburied marbles, number of ≧⅔ buried marbles
and number of totally buried (mean, SEM) in LL-Wat, SL-Wat
and SL-Almo groups. Statistical analyses: 1-factor ANOVAs:
p values for the effect of L/D cycle and post-hoc comparisons
by Fisher's PLSD test.

| | | | ≧⅔ | |
|---|---|---|---|---|
| | | Unburied | buried | Tot buried |
| LL-Wat | Mean | 3.8 | 17.8 | 10.2 |
| | SEM | 1.4 | 2.2 | 3.2 |
| SL-Wat | Mean | 10.3 | 8.0 | 3.3 |
| | SEM | 1.1 | 1.6 | 1.5 |
| Fisher's PLSD test | p vs. LL-Wat ≦ | 0.05 | 0.05 | NS |
| SL-Almo | Mean | 5.3 | 16.3 | 8.9 |
| | SEM | 1.6 | 2.2 | 2.0 |
| Fisher's PLSD test | p vs. LL-Wat ≦ | 0.6 | 0.7 | NS |
| | p vs. SL-Wat ≦ | 0.05 | 0.05 | NS |
| 1-Factor ANOVA | p ≦ | 0.05 | 0.05 | 0.4 |

FIG. 23 shows the number of unburied marbles and FIG. 24 shows the number of ≧⅔ buried marbles, both figure for LL-Wat, SL-Wat and SL-Almo groups. The results are expressed as mean±SEM. Statistical analyses by Fisher's PLSD tests: difference vs. difference vs. LL-Wat group: * p≦0.05; difference vs. SL-Wat group: §p≦0.05.

Body Weight

The analyses of body weight can be summarized as follows.

The body weight gain was increased by long light condition and was decreased by short light condition.

In vehicle-treated animals, the body weight gain was lower when vehicle was water administered PO than when it was DMSO administered IP in Control (12 h/12 h L/D) condition, but not in long light and in short light conditions.

Almorexant reduced the decrease of body weight induced by short light condition.

The results are detailed below:

Initial Body Weight Before Initiation of L/D Change

There were no significant difference of body weight between groups on day d-4, before the initiation of L/D change, whatever the groups considered were:
1) C-DMSO, C-Wat, LL-DMSO, LL-Wat, SL-DMSO, SL-Wat, SL-TCS, SL-Almo (Table 25; ANOVA: p=NS), or
2) C-Veh, LL-Veh, SL-Veh, SL-TCS, SL-Almo (ANOVA: p=NS).

However, comparisons between groups of the body weight before initiation of L/D change by Student's t-test showed a difference between SL-DMSO group (BW=158.3±5.2 g) and SL-Wat group (BW=142.1±4.2 g) (p≦0.05) and a close-to-significant difference between SL-Almo group (BW=150.7±2.5 g) and SL-Wat group (BW=142.1±4.2 g) (0.05<p≦0.10).

Therefore, the following analyses were performed on the BW expressed in percentage of the initial BW (on day d-4 before L/D change).

TABLE 25
Body weight (in grams (g), mean, SEM) before the initiation of L/D change
(day D−4) in C-DMSO, C-Wat, LL-DMSO, LL-Wat, SL-DMSO, SL-TCS SL-Wat and SL-
Almo groups. Statistical comparisons: between tests comparisons by Student's t-test
(given only for information since the ANOVA showed no overall difference between
groups, see text).

| | | d−4 | | | d−4 | | | d−4 |
|---|---|---|---|---|---|---|---|---|
| C-DMSO | Mean | 149.8 | SL-DMSO | Mean | 158.3 | SL-Wat | Mean | 142.1 |
| | SEM | 4.7 | | SEM | 5.2 | | SEM | 4.2 |
| C-Wat | Mean | 151.1 | | p vs. C-DMSO | 0.255 | | p vs. C-Wat | 0.102 |
| | SEM | 2.7 | | p vs. LL-DMSO | 0.122 | | p vs. LL-Wat | 0.195 |
| | p vs. C-DMSO | 0.828 | SL-TCS | Mean | 150.4 | | p vs. SL-DMSO | 0.036 |
| LL-DMSO | Mean | 148.8 | | SEM | 3.1 | SL-Almo | Mean | 150.7 |
| | SEM | 2.1 | | p vs. C-DMSO | 0.926 | | SEM | 2.5 |
| | p vs. C-DMSO | 0.845 | | p vs. LL-DMSO | 0.741 | | p vs. C-Wat | 0.925 |
| LL-Wat | Mean | 151.6 | | p vs. SL-DMSO | 0.178 | | p vs. LL-Wat | 0.865 |
| | SEM | 5.4 | | | | | p vs. SL-wat | 0.077 |
| | p vs. C-Wat | 0.936 | | | | | | |
| | p vs. LL-DMSO | 0.645 | | | | | | |

Effect of L/D Changes on Body Weight

The body weight expressed in percentage of the initial BW of C-Veh, LL-Veh, SL-Veh, SL-TCS and SL-Almo are presented in Table 26 (C-Veh, LL-Veh and SL-Veh only).

Comparisons of Veh groups (C-Veh, LL-Veh, SL-Veh) by ANOVAs for repeated measures show:
1) Comparison of the three groups (C-Veh, LL-Veh, SL-Veh:
   a) A significant difference between groups (p≦0.01).
   b) A significant difference between days (p≦0.001).
   c) A significant interaction groups×days (p≦0.001).
CONCLUSION: There was a significant increase of BW. The BW gain was different between the three L/D conditions groups.
2) Comparison of C-Veh and LL-Veh:
   a) A significant difference between groups (p≦0.05).
   b) A significant difference between days (p≦0.001).
   c) A significant interaction groups×days (p≦0.001).

CONCLUSION: The BW gain was increased by 4.3% in Long Light condition in comparison with Control condition.
1) Comparison of C-Veh and LL-Veh:

a) A No significant difference between groups (p≦0.10).

b) A significant difference between days (p≦0.001).

c) A significant interaction groups×days (p≦0.001).

CONCLUSION: The BW gain was decreased by 3.0% in Short Light condition in comparison with Control condition.
2) Comparison of SL-Veh and LL-Veh:
   a) A significant difference between groups (p≦0.01).
   b) A significant difference between days (p≦0.001).
   c) A significant interaction groups×days (p≦0.001).

CONCLUSION: The BW gain was decreased by 7.0% in Short Light condition in comparison with Long Light condition.

TABLE 26
Body weight in percent of initial body weight (d−4) (mean, SEM) on d−4 and
from d1 to d26 (before treatment, top panel) and from d29 to d54 (during treatment,
bottom panel) in C-Veh, LL-Veh, SL-Veh, SLT-TCS and SL-Almo groups.

| | | d−4 | d1 | d3 | d5 | d8 | d10 | d12 | d15 | d17 | d19 | d22 | d24 | d26 |
|---|---|---|---|---|---|---|---|---|---|---|---|---|---|---|
| C-Veh | Mean | 100.0 | 112.5 | 118.1 | 123.9 | 127.1 | 131.2 | 136.3 | 140.9 | 145.0 | 148.0 | 150.9 | 151.8 | 157.5 |
| | SEM | 0.0 | 0.8 | 1.0 | 1.1 | 1.0 | 1.1 | 1.2 | 1.6 | 1.6 | 1.6 | 2.0 | 1.9 | 2.0 |
| LL-Veh | Mean | 100.0 | 113.0 | 119.9 | 126.7 | 130.5 | 134.3 | 139.6 | 144.9 | 149.6 | 153.9 | 157.0 | 160.3 | 164.0 |
| | SEM | 0.0 | 0.8 | 1.1 | 1.5 | 1.2 | 1.4 | 1.3 | 1.4 | 1.7 | 1.8 | 1.7 | 1.8 | 1.6 |
| SL-Veh | Mean | 100.0 | 112.1 | 117.7 | 124.1 | 128.0 | 131.9 | 136.8 | 140.2 | 143.3 | 147.0 | 148.9 | 153.0 | 154.3 |
| | SEM | 0.0 | 0.8 | 0.8 | 1.0 | 1.1 | 1.2 | 1.2 | 1.8 | 1.5 | 1.5 | 1.7 | 1.6 | 1.9 |
| SL-TCS | Mean | 100.0 | 113.5 | 119.5 | 126.8 | 131.2 | 134.5 | 139.6 | 142.6 | 146.4 | 148.6 | 152.1 | 154.6 | 155.4 |
| | SEM | 0.0 | 0.7 | 0.9 | 1.1 | 1.4 | 1.3 | 1.7 | 1.9 | 1.7 | 1.9 | 2.0 | 2.1 | 1.9 |
| SL-Almo | Mean | 100.0 | 114.5 | 121.4 | 128.3 | 133.3 | 137.4 | 144.2 | 146.8 | 150.7 | 154.5 | 155.7 | 160.5 | 160.0 |
| | SEM | 0.0 | 2.5 | 2.8 | 3.0 | 3.1 | 3.5 | 3.9 | 3.4 | 3.5 | 3.3 | 3.2 | 2.3 | 3.4 |

| | | d29- | d31- | d33- | d36- | d38- | d40- | d43- | d45- | d47- | d50- | d52- | d54- |
|---|---|---|---|---|---|---|---|---|---|---|---|---|---|
| | | t1 | t3 | t5 | t8 | t10 | t12 | t15 | t17 | t19 | t22 | t24 | t26 |
| C-Veh | Mean | 163.8 | 166.6 | 166.4 | 168.5 | 169.0 | 171.7 | 174.8 | 175.9 | 177.3 | 177.4 | 180.2 | 180.0 |
| | SEM | 2.0 | 2.1 | 2.3 | 2.1 | 2.2 | 2.2 | 2.1 | 1.9 | 2.1 | 2.1 | 2.7 | 2.2 |
| LL-Veh | Mean | 167.5 | 170.4 | 172.1 | 175.0 | 177.6 | 178.1 | 180.2 | 183.2 | 183.8 | 185.4 | 186.1 | 187.8 |
| | SEM | 1.8 | 1.8 | 1.9 | 2.6 | 2.8 | 2.8 | 2.7 | 3.0 | 3.3 | 2.9 | 3.2 | 2.8 |
| SL-Veh | Mean | 159.9 | 161.7 | 163.0 | 166.5 | 168.0 | 167.7 | 171.7 | 170.8 | 172.3 | 173.0 | 174.7 | 174.6 |
| | SEM | 1.9 | 1.9 | 2.0 | 2.1 | 2.4 | 2.2 | 2.2 | 2.0 | 1.8 | 2.2 | 1.7 | 1.9 |
| SL-TCS | Mean | 162.1 | 161.0 | 165.8 | 168.2 | 169.5 | 168.9 | 171.4 | 172.9 | 173.2 | 173.9 | 176.9 | 174.7 |
| | SEM | 2.1 | 1.9 | 2.3 | 2.4 | 2.6 | 2.7 | 2.5 | 2.6 | 2.7 | 2.6 | 2.9 | 2.7 |
| SL-Almo | Mean | 164.4 | 167.0 | 168.8 | 173.4 | 175.5 | 176.6 | 179.7 | 181.7 | 183.5 | 185.5 | 187.5 | 187.0 |
| | SEM | 3.4 | 3.7 | 3.5 | 3.8 | 4.1 | 3.8 | 3.9 | 4.0 | 4.3 | 4.4 | 4.3 | 4.5 |

Effect of Treatments and L/D Changes on Body Weight

Effect of Vehicle

The body weight expressed in percentage of the initial BW of C-DMSO, C-Wat, LL-DMSO, LL-Wat, SL-DMSO and SL-Wat are presented in Table 27.

Comparisons C-DMSO, C-Wat, LL-DMSO, LL-Wat, SL-DMSO and SL-Wat groups by 2-factor ANOVA for repeated measures show:
1) No significant effect of vehicle (p>0.30).
2) A significant effect of L/D condition (p≦0.01).
3) A significant difference between days (p≦0.001).
4) No significant interaction vehicle×L/D condition (p>0.10)
5) No significant interaction vehicle×day (p>0.30)
6) A significant interaction L/D condition×day (p≦0.05)
7) A significant interaction vehicle×L/D condition×day (p≦0.05).

Comparisons C-DMSO, C-Wat, LL-DMSO, LL-Wat, SL-DMSO and SL-Wat groups by ANOVA for repeated measures show:
8) Comparison C-DMSO and C-Wat:
   a) No significant effect of vehicle (p>0.30).
   b) A significant difference between days (p≦0.001).
   c) A significant interaction vehicle×day (p≦0.01)

CONCLUSION: In Control condition, the BW gain was lower when vehicle was water than when vehicle was DMSO.
9) Comparison LL-DMSO and LL-Wat:
   a) No significant effect of vehicle (p>0.30).
   b) A significant difference between days (p≦0.001).
   c) No significant interaction vehicle×day (p>0.30)

CONCLUSION: In Long light condition, the BW gain was not different depending on whether the vehicle was water or DMSO.
10) Comparison SL-DMSO and SL-Wat:
   a) No significant effect of vehicle (p>0.10).
   b) A significant difference between days (p≦0.001).
   c) No significant interaction vehicle×day (p>0.20)

CONCLUSION: In Short light condition, the BW gain was not different depending on whether the vehicle was water or DMSO.

TABLE 27
Body weight in percent of initial body weight (d−4) (mean, SEM) from d29 to
d54 in C-DMSO, C-Wat, LL-DMSO, LL-Wat, SL-DMSO, SLT-TCS, SL-Wat and SL-
Almo groups.

| | | d29- | d31- | d33- | d36- | d38- | d40- | d43- | d45- | d47- | d50- | d52- | d54- |
|---|---|---|---|---|---|---|---|---|---|---|---|---|---|
| | | t1 | t3 | t5 | t8 | t10 | t12 | t15 | t17 | t19 | t22 | t24 | t26 |
| C-DMSO | Mean | 164.1 | 166.8 | 167.9 | 170.4 | 170.3 | 172.9 | 176.8 | 177.3 | 179.9 | 180.2 | 184.9 | 183.9 |
| | SEM | 3.4 | 3.9 | 3.7 | 3.7 | 4.0 | 4.1 | 3.7 | 3.6 | 3.7 | 3.8 | 4.7 | 3.7 |
| C-Wat | Mean | 163.5 | 166.4 | 164.9 | 166.5 | 167.7 | 170.5 | 172.7 | 174.4 | 174.8 | 174.7 | 175.4 | 176.1 |
| | SEM | 2.2 | 2.1 | 3.0 | 2.0 | 2.4 | 1.9 | 1.8 | 1.4 | 1.7 | 1.5 | 1.4 | 0.9 |
| LL-DMSO | Mean | 166.7 | 168.8 | 171.7 | 172.5 | 174.8 | 174.8 | 176.3 | 180.5 | 179.9 | 182.7 | 183.5 | 184.8 |
| | SEM | 0.9 | 0.6 | 1.4 | 2.0 | 1.6 | 0.7 | 1.9 | 2.3 | 2.9 | 2.7 | 3.4 | 2.3 |
| LL-Wat | Mean | 168.2 | 171.9 | 172.4 | 177.6 | 180.4 | 181.4 | 184.1 | 185.9 | 187.7 | 188.1 | 188.6 | 190.8 |
| | SEM | 3.6 | 3.6 | 3.8 | 4.8 | 5.4 | 5.5 | 4.7 | 5.6 | 5.7 | 5.1 | 5.5 | 5.2 |
| SL-DMSO | Mean | 158.1 | 158.8 | 161.1 | 163.8 | 165.3 | 164.3 | 167.9 | 168.1 | 168.4 | 168.7 | 171.2 | 170.8 |
| | SEM | 2.1 | 0.7 | 1.7 | 2.1 | 2.3 | 1.1 | 1.5 | 1.5 | 1.4 | 1.0 | 1.1 | 0.8 |
| SL-Wat | Mean | 162.6 | 163.5 | 165.2 | 169.3 | 170.8 | 169.6 | 174.0 | 173.5 | 174.0 | 174.5 | 177.4 | 175.1 |
| | SEM | 2.5 | 3.1 | 2.7 | 3.2 | 3.3 | 3.5 | 3.2 | 2.2 | 2.2 | 2.7 | 2.5 | 2.8 |

Effect of TCS 1102 and of Almorexant

Because the results presented above showed that the BW may be different depending on whether the vehicle was DMSO administered IP or water administered PO, the effect of TCS 1102 was analyzed by comparison of groups administered IP and the effect of almorexant was analyzed by comparisons of groups administered PO.

The body weight expressed in percentage of the initial BW of C-DMSO, C-Wat, LL-DMSO, LL-Wat, SL-DMSO, SL-TCS, SL-Wat and SL-Almo groups are presented in Table 28 (C-DMSO, LL-DMSO, SL-DMSO and SL-TCS groups) and (C-Wat, LL-Wat, SL-Wat and SL-Almo) groups.

Comparisons C-DMSO, LL-DMSO, SL-DMSO and SL-TCS groups by ANOVA for repeated measures show:
1) Comparison of the four groups:
   a) A significant difference between groups (p≦0.05).
   b) A significant difference between days (p≦0.001).
   c) A near-significant interaction group×day (0.05<p≦0.10)

CONCLUSION: In groups which received treatment IP, the BW was different between groups.

Comparisons C-Wat, LL-Wat, SL-Wat and SL-Almo groups by ANOVA for repeated measures show:
2) Comparison of the four groups:
   a) No significant difference between groups (p>0.20).
   b) A significant difference between days (p≦0.001).
   c) A significant interaction group×day (p≦0.001)

CONCLUSION: In groups which received treatment PO, the BW gain was different between groups.
4) Comparison of the SL-Wat and SL-Almo groups:
   a) No significant difference between groups (p>0.30).
   b) A significant difference between days (p≦0.001).
   c) A significant interaction group×day (p≦0.01)

CONCLUSION: Almorexant increased the BW, i.e. reduced the BW decrease induced by short light condition.

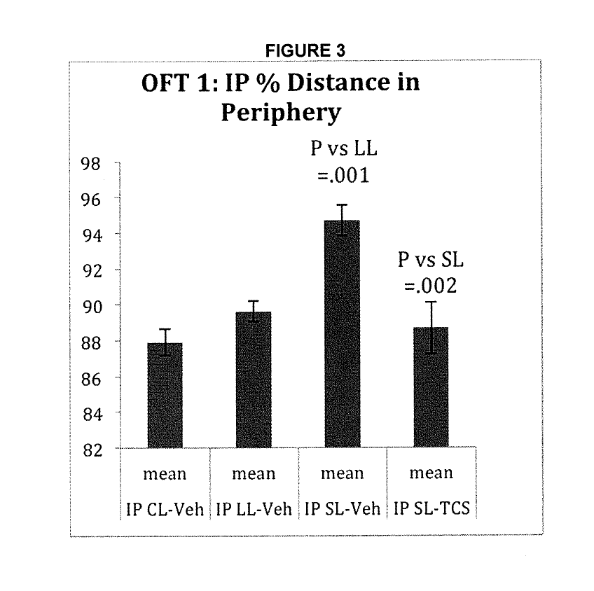

### Summary of FIGS. 3, 4, 5, 6, 10, 13, 14, 15, 16, 17, 18, 19, 20, 21 and 22.

### IP:

### OFT:

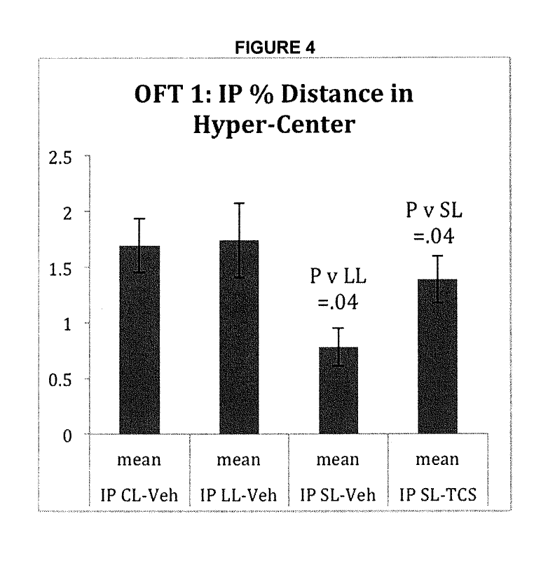

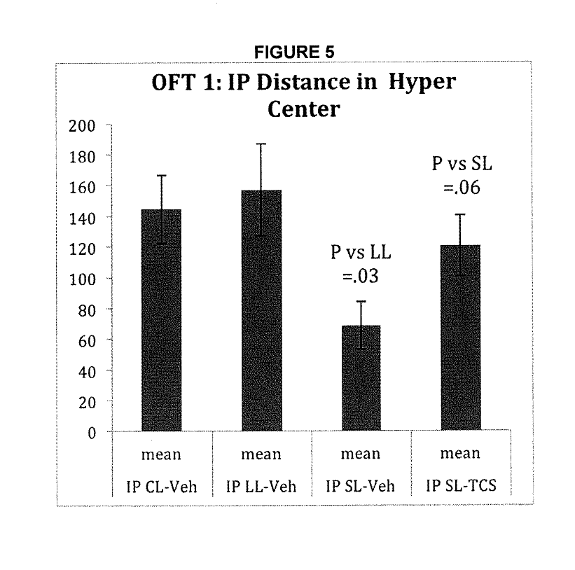

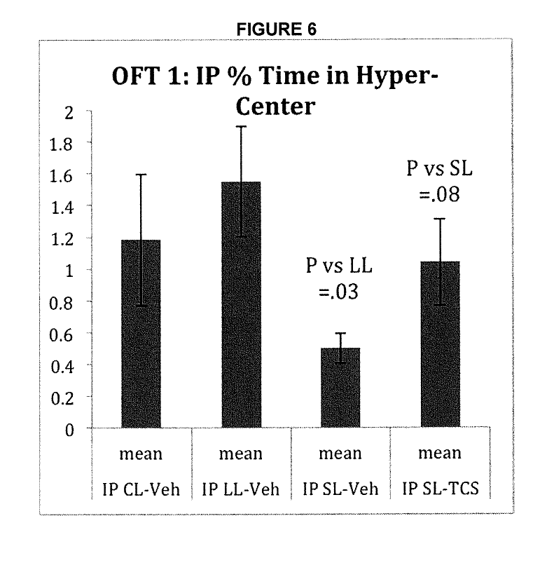

SL-Veh increased thigmotactic anxiety-like behaviors (FIG. 3) which were treated fully and with statistical significance with TCS 1102. SL-Veh decreased exploratory behaviors (FIG. 4) which were treated with statistical significance with TCS 1102. Another parameter of exploratory behavior was measured in FIGS. 5 and 6, which was similarly treated with TCS 1102.

### OG:

### EPM:

FIG. 10 demonstrates that increased anxiety-like behaviors, which also may be interpreted as decreased exploratory behaviors, were increased with statistical significance in the SL-Veh group and treated with statistical significance with almorexant.

### FST:

FIG. 13 demonstrates the statistically significant anti-depressant and anti-despair effect of Almorexant treatment in SL treated rats. FIG. 14 corroborates with the findings from FIG. 13.

FIG. 15 demonstrates the statistically significant antidepressant and anti-despair effects of LL compared to CL, which is corroborated by FIGS. 16 and 17. FIG. 18 demonstrates that SL tended to increase floating, which approximates behavioral despair and depression-like characteristics, which is corroborated by FIG. 19.

### MBT:

FIG. 20 demonstrates an inability to deal with slight stressors in the environment in the SL condition compared to the LL condition. This effect of SL is treated with statistical significance with almorexant. FIG. 21 demonstrates a statistically significant increase in hypomanic-like tendencies in the SL-Veh group. This increase was treated with statistical significance with almorexant.

FIG. 22 demonstrates the effectiveness of almorexant in treating SL-induced increases in marbles slightly buried. Marbles may only be slightly buried because of a SL-induced inability to remove slight stressors from the environment and/or a SL-induced decrease in mobility. Both of these possibilities are translational to human SAD, indicating the effectiveness of orexin receptor antagonists in treating human SAD symptoms.

TABLE 28
Body weight in percent of initial body weight (d−4) (mean, SEM) from d29 to
d54 in C-DMSO, C-Wat, LL-DMSO, LL-Wat, SL-DMSO, SLT-TCS, SL-Wat and SL-
Almo groups.

| | | | d29- | d31- | d33- | d36- | d38- | d40- | d43- | d45- | d47- | d50- | d52- | d54- |
|---|---|---|---|---|---|---|---|---|---|---|---|---|---|---|
| | | | t1 | t3 | t5 | t8 | t10 | t12 | t15 | t17 | t19 | t22 | t24 | t26 |
| C-Veh | C-DMSO | Mean | 164.1 | 166.8 | 167.9 | 170.4 | 170.3 | 172.9 | 176.8 | 177.3 | 179.9 | 180.2 | 184.9 | 183.9 |
| | | SEM | 3.4 | 3.9 | 3.7 | 3.7 | 4.0 | 4.1 | 3.7 | 3.6 | 3.7 | 3.8 | 4.7 | 3.7 |
| | C-Wat | Mean | 163.5 | 166.4 | 164.9 | 166.5 | 167.7 | 170.5 | 172.7 | 174.4 | 174.8 | 174.7 | 175.4 | 176.1 |
| | | SEM | 2.2 | 2.1 | 3.0 | 2.0 | 2.4 | 1.9 | 1.8 | 1.4 | 1.7 | 1.5 | 1.4 | 0.9 |
| LL-Veh | LL-DMSO | Mean | 166.7 | 168.8 | 171.7 | 172.5 | 174.8 | 174.8 | 176.3 | 180.5 | 179.9 | 182.7 | 183.5 | 184.8 |
| | | SEM | 0.9 | 0.6 | 1.4 | 2.0 | 1.6 | 0.7 | 1.9 | 2.3 | 2.9 | 2.7 | 3.4 | 2.3 |
| | LL-Wat | Mean | 168.2 | 171.9 | 172.4 | 177.6 | 180.4 | 181.4 | 184.1 | 185.9 | 187.7 | 188.1 | 188.6 | 190.8 |
| | | SEM | 3.6 | 3.6 | 3.8 | 4.8 | 5.4 | 5.5 | 4.7 | 5.6 | 5.7 | 5.1 | 5.5 | 5.2 |
| SL-Veh IP | SL-DMSO | Mean | 158.1 | 158.8 | 161.1 | 163.8 | 165.3 | 164.3 | 167.9 | 168.1 | 168.4 | 168.7 | 171.2 | 170.8 |
| | | SEM | 2.1 | 0.7 | 1.7 | 2.1 | 2.3 | 1.1 | 1.5 | 1.5 | 1.4 | 1.0 | 1.1 | 0.8 |
| | SL-TCS | Mean | 162.1 | 161.0 | 165.8 | 168.2 | 169.5 | 168.9 | 171.4 | 172.9 | 173.2 | 173.9 | 176.9 | 174.7 |
| | | SEM | 2.1 | 1.9 | 2.3 | 2.4 | 2.6 | 2.7 | 2.5 | 2.6 | 2.7 | 2.6 | 2.9 | 2.7 |
| SL-Veh PO | SL-Wat | Mean | 161.7 | 164.7 | 164.9 | 169.1 | 170.8 | 171.2 | 175.5 | 173.6 | 176.2 | 177.3 | 178.1 | 178.3 |
| | | SEM | 3.2 | 3.5 | 3.5 | 3.5 | 4.0 | 3.9 | 3.6 | 3.5 | 2.6 | 3.5 | 2.7 | 3.1 |
| | SL-Almo | Mean | 164.4 | 167.0 | 168.8 | 173.4 | 175.5 | 176.6 | 179.7 | 181.7 | 183.5 | 185.5 | 187.5 | 187.0 |
| | | SEM | 3.4 | 3.7 | 3.5 | 3.8 | 4.1 | 3.8 | 3.9 | 4.0 | 4.3 | 4.4 | 4.3 | 4.5 |

Growth Rate Graphs

Though seasonal changes in metabolism may present differently in Wistar rats and humans, they are both caused by seasonal light and radiation changes. Accordingly, what is translational from a rat to a human model is how DORAs work to counteract what seasonal changes in growth and metabolism do occur in rats. The growth rate effects of the photoperiods were likely caused by a biological mechanism. It would have been expected that the SL rats, who have a longer active phase, would have had had more time to eat than the LL rats, giving them an increased growth rate. However, despite having a longer time to feed, the SL rats consistently had lower growth rates than LL. The effects of the drugs confirmed this. Each of the following graphs that demonstrate a change in rat metabolism under the SL photoperiod compared to the LL photoperiod that is treated fully or partially by a DORA is evidence for the effectiveness of DORAs in treating human metabolic seasonality. This effect was found for both TCS 1102 and almorexant. Consequently the exemplary research indicates the effectiveness of orexin receptor antagonism for the treatment of seasonal changes in human metabolism. These changes in humans may present as, but are not limited to seasonal carbohydrate cravings, increased appetite, increased hunger, and weight gain.

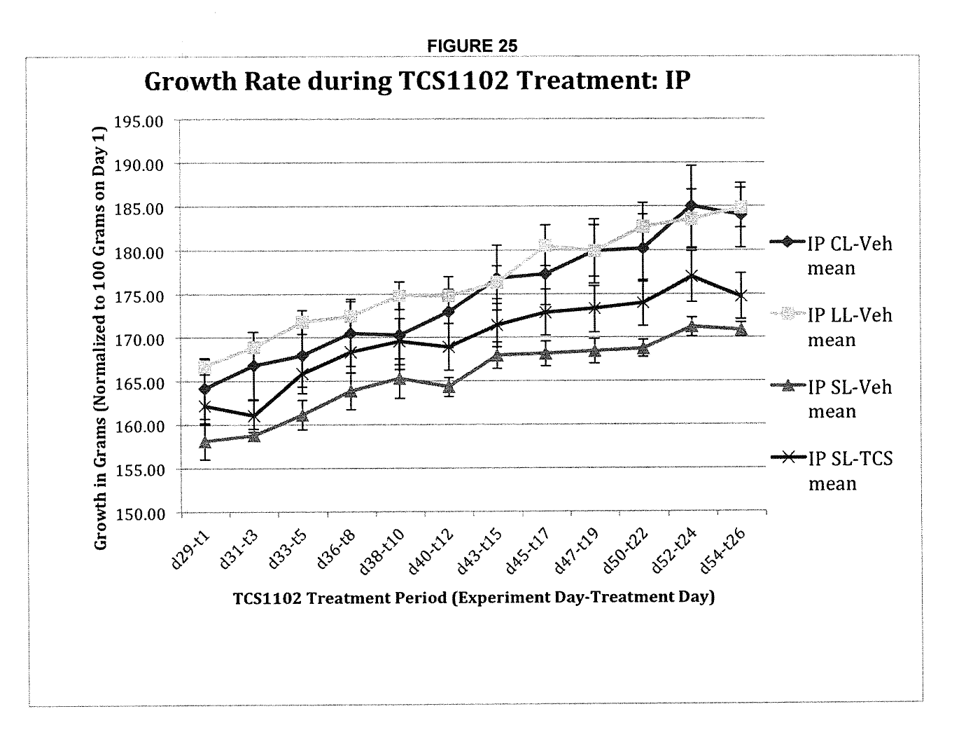

FIG. 25: Growth rates during TCS1102 treatment in the IP groups. CL and LL were found to have the highest growth rates with a diminished growth rate for SL-Veh and SL-TCS. TCS treatment lessened the SL-induced low growth rate.

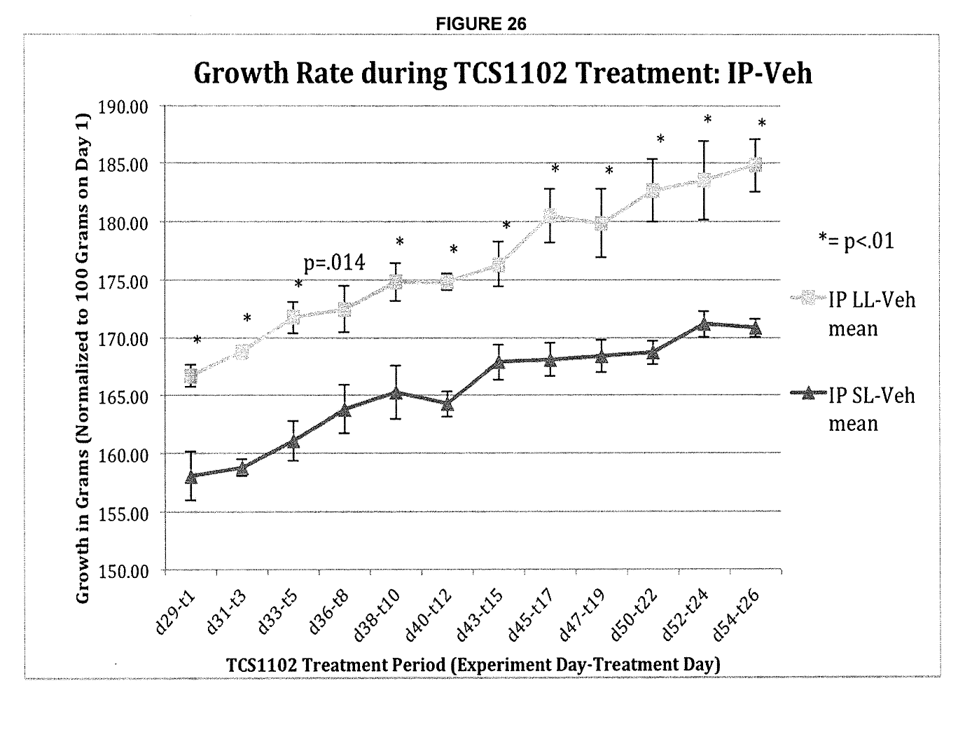

FIG. 26: Growth rates of the SL-Veh vs LL-Veh groups. LL had a consistently statistically significant increase in growth rate.

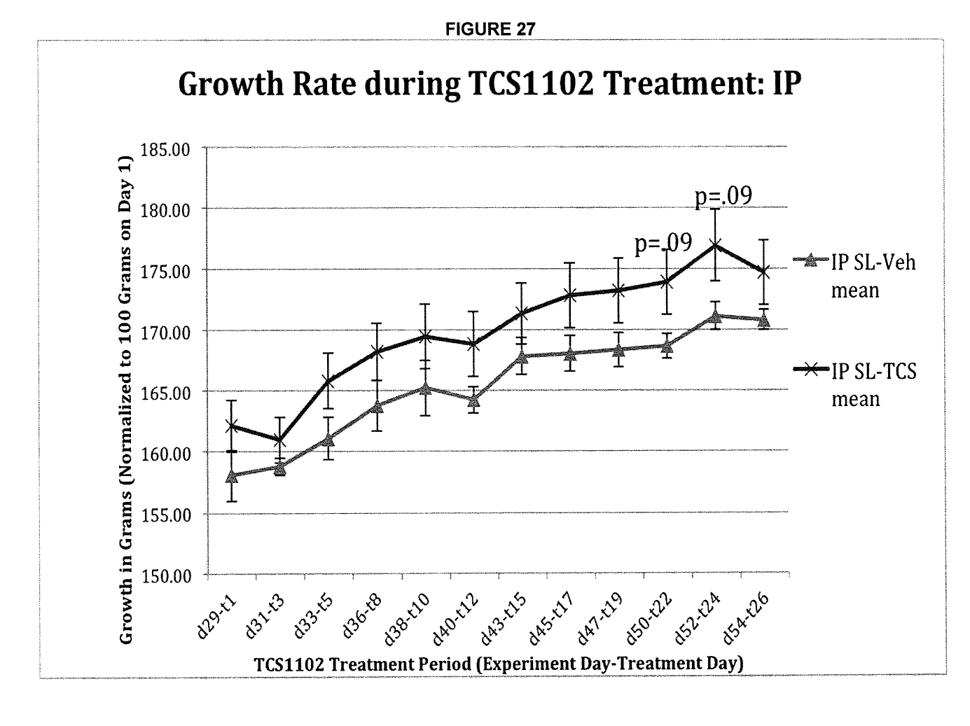

FIG. 27: Growth rates of SL-Veh vs SL-TCS groups to determine the effect of IP DORA treatment. TCS-1102 consistently increased the growth rate compared to SL-Veh. As LL and CL had the highest growth rates, the increased growth rates in the TCS group indicate a partial reversion to the LL characteristics.

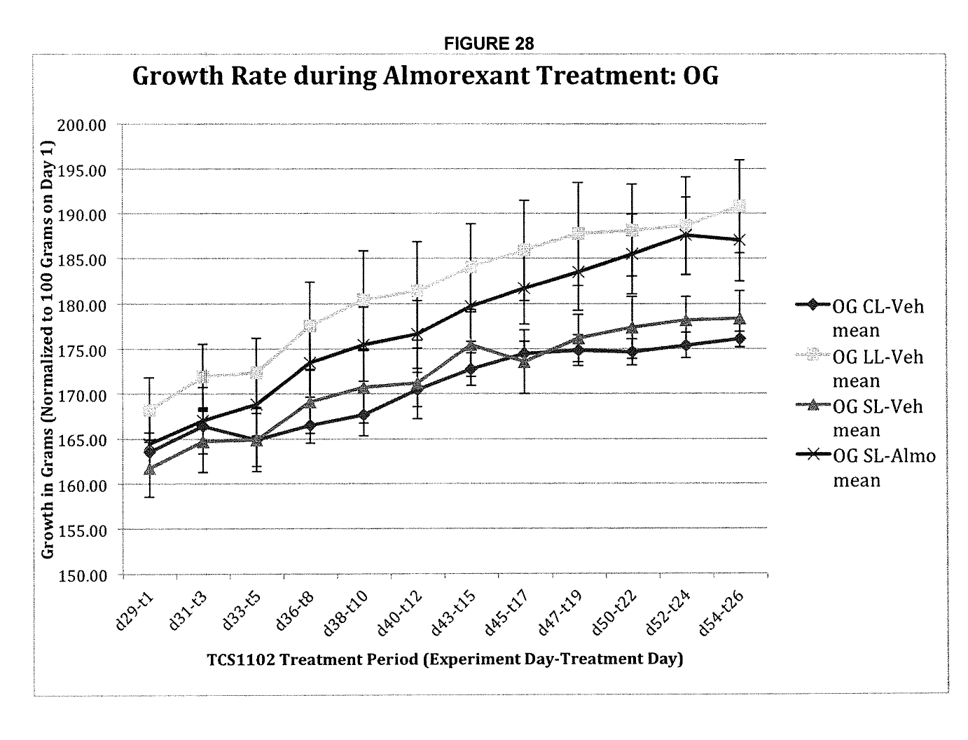

FIG. 28: Growth rate during OG almorexant treatment. LL had the highest growth rate while SL-Veh and CL-Veh growth rates were diminished. CL does not approximate natural seasonal photoperiods, but may indicate that CL induces a background growth rate depression in rats. This graph also shows that DORA treatment induces almost full remission back to the LL growth rate.

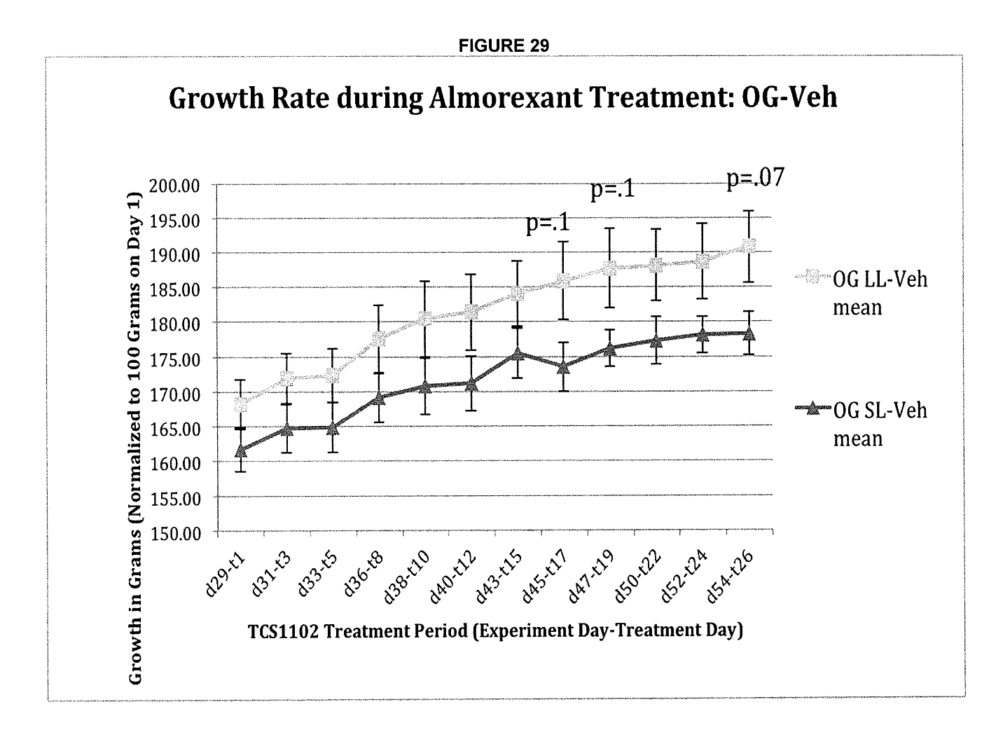

FIG. 29: Growth rate of LL-Veh vs SL-Veh. LL-Veh consistently had a higher growth rate compared to SL-Veh.

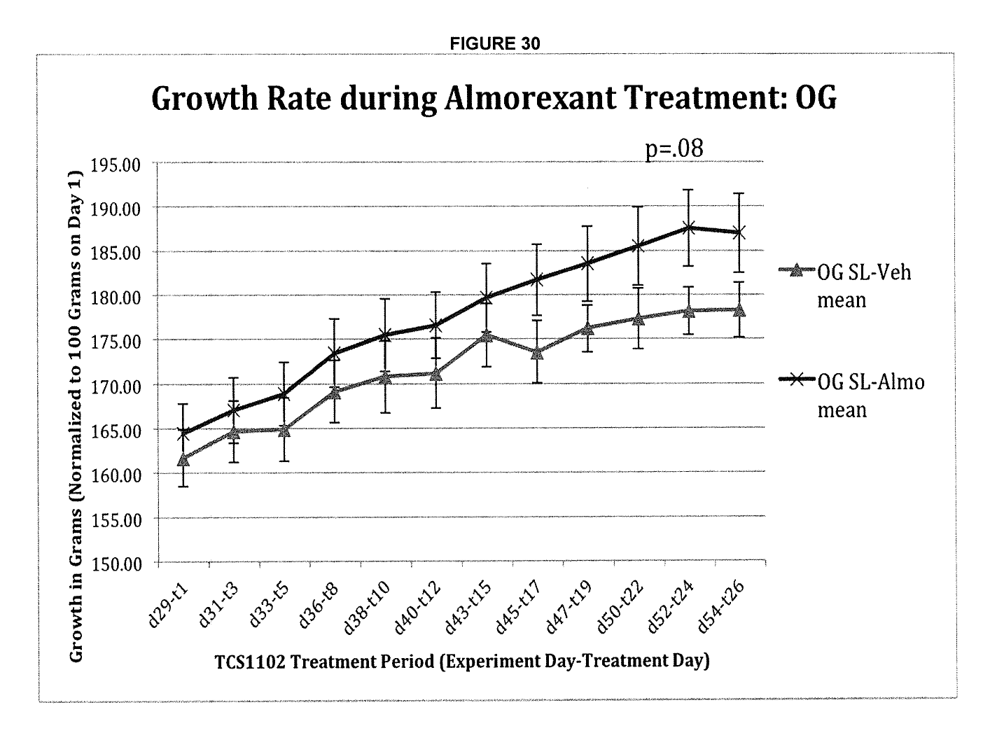

FIG. 30: OG almorexant treatment was consistently found to increase growth rate back to LL levels. In fact, there was no statistical significance between the LL and SL-Almo groups, indicating the effectiveness of DORA treatment to treat SL or SAD symptoms.

### Discussion of Experimental Results

The injection of DMSO induced:
1) In the open-field test: a close-to-significant anxiogenic-like effect at the sessions in SL condition, but not in C and LL conditions.
2) In the EPM test: an anxiogenic-like effect which was significant in LL condition, close to significance in SL condition and which was not observed in C condition, and decrease in activity which was significant only in LL condition.
3) In the FST: a close-to-significant depressant-like effect observed only in C condition and at the second session.
4) In the marble burying test: decrease in the number of marbles buried in LL condition only, which may show an anxiolytic-like effect but which more likely show a decrease in activity.
5) An increase of body weight gain in C condition only.

Consequently, the results were analyzed separately on IP administered groups (DMSO, TCS 1102) and on PO administered groups (water, Almorexant).

A selection of the effects of L/D changes, of TCS 1102 and of Almorexant shows that:
1) Long light:
   a) Induced an anxiolytic-like effect and an increase in activity in the EPM test in water-treated rats, but not in DMSO-treated rats.
   b) Also induced a near significant increase in activity in the open-field at the first session, but not at the second session, in water-treated rats only.
   c) Did not significantly modify anxiety in the open-field test.
2) Short light:
   a) Induced an anxiogenic-like effect in DMSO-treated rats, but not in water-treated rats, in the EPM test and in the open-field test at the first session only.
   b) Did not significantly modify activity in the EPM test and in the open-field test.
   c) Induced a decrease in the number of marbles buried, i.e. an anxiolytic-like effect in the marble burying test in water-treated rats, but not in DMSO-treated rats. However, this effect is questionable: it is more likely that the decrease of the number of marbles buried resulted from a non-specific decrease in activity induced by short light.
3) TCS 1102:
   a. Treated SL-induced anxiogenic-like decrease in exploratory behavior in the OFT.
   b. Treated increases in anxiogenic thigmotaxis in the OFT caused by SL.
   c. Tended to reduce the anxiogenic-like effect of short light in the EPM test.
   d. Vehicle groups for LL and SL demonstrated that SL is characterized by a decreased growth rate. TCS 1102 increased the growth rate of rats exposed to SL, reverting growth rates in the direction of rats exposed to LL. This indicates the potential effectiveness of orexin receptor antagonists for treating human seasonal changes in metabolism.
4) Almorexant:
   a. Tended to reduce the decrease in mobility caused by the SL in the OFT. The decreased mobility may be increased anxiogenic-like behavior and/or decreased exploratory behavior.
   b. Tended to decrease the anxiogenic-like and non-exploratory behavior of the SL compared to the LL in the EPM. Similarly almorexant tended to increase exploratory behavior.
   c. In the EPM SL group, almorexant increased exploratory behavior and mobility, making the LL and SL-Almo groups statistically indistinguishable, as would be expected for a SAD-treating drug.
   d. In the FST, treated the background SAD-like and depression-like symptoms in the SL rats. Almorexant decreased immobility time from SL to LL levels, and increased SL climbing time. In rats exposed to SL, Almorexant induced a very strong behavioral effect that mimicked the effects the LL
   e. In the MBT, almorexant was very effective in treating the significant effects of SL, which was characterized by decreased marble burying depths and increased total unburied marbles.
   f. Similar to the effects of TCS 1102, almorexant increased the SL growth rate so that SL-Almo and LL groups were statistically indistinguishable. The SL-Veh group had a lower growth rate than the LL-Veh group. This indicates the effectiveness of orexin receptor antagonists in treating human seasonal metabolic symptoms.

### EXPERIMENTAL CONCLUSIONS

A progressive lengthening of the daylight duration induced a strong behavioral and physiological profile, which included but was not limited to anxiolytic, anti-depressant, and anti-hypomanic-like effects, and increased growth rate. These effects are consistent with past studies, which showed that long photoperiod produce antidepressant-like effect in nocturnal rats. Conversely, a progressive shortening of the daylight duration induced an anxiogenic-like effect, an increase in hypomanic-like tendencies, a decrease in growth rate, and other behaviors and metabolic changes.

These effects were dependent on other manipulations, which may induce slight stress, such as the administration of vehicles. Therefore, because TCS 1102 and almorexant were administered by different routes and in different vehicles, it is difficult to compare their effects. Despite the differing routes of administration, TCS and almorexant both were found to reduce the effects of SL and/or mimic the effects of LL.

The present study showed that TCS 1102 and almorexant both treated the effects of a shortening of daylight duration and/or induced an effect similar to LL.

Almorexant and TCS 1102 performed differently in the behavioral and physiological measures tested in the exemplary research. This is likely due to their different pharmacokinetics, antagonism constants, ratios of OxR1 to OxR2 antagonism, and pharmacological profiles. Also, there is evidence that almorexant may antagonize the orexin receptors for longer than TCS 1102. Given that both drugs were administered at the same time via different administration routes, this likely had an effect on behavioral results. What is striking, however, is that both drugs had the ability to either treat SL-induced behavioral symptoms and/or recreate LL behavioral profiles, as CL likely induces a background SAD-like effect on Wistar rats. This indicates the potential effectiveness of orexin receptor antagonists for treating SAD in humans.

While only a few exemplary embodiments of this invention have been described in detail, those skilled in the art will recognize that there are many possible variations and modifications, which may be made in the exemplary embodiments while yet retaining many of the novel and advantageous features of this invention. Accordingly, it is intended that the following claims cover all such modifications and variations.

1. A method for reducing one or more symptoms associated with seasonal affective disorder in an individual, the method comprising administering to the individual suffering from seasonal affective disorder, a composition comprising a therapeutically effective amount of an agent to down regulate the activity of an orexin selected from the group consisting of orexin A or orexin B, at one or more orexin receptors.

2. The method of claim 1, wherein the symptoms associated with seasonal affective disorder are selected from the group consisting of depression, anxiety, lethargy, hypersomnia, tiredness, aches, carbohydrate and other food cravings, weight gain and atypical symptoms of depression.

3. The method of claim 1 wherein orexin receptor activity is down regulated at an orexin 1 receptor.

4. The method of claim 1 wherein orexin receptor activity is down regulated at an orexin 2 receptor.

5. The method of claim 1 wherein the orexin receptor activity is down regulated at both orexin 1 and orexin 2 receptors.

6. The method of claim 1 wherein expression of an orexin receptor is decreased.

7. The method of claim 1 where orexin receptor activity is down regulated through inactivation of orexin A.

8. The method of claim 1 where orexin receptor activity is down regulated through inactivation of orexin B.

9. The method of claim 1 wherein the agent comprises an orexin receptor antagonist selected from the group consisting of an orexin receptor antagonist selective for orexin 1 receptors, an orexin receptor antagonist selective for orexin 2 receptors, and an orexin receptor antagonist having affinity at orexin 1 receptors and orexin 2 receptors.

10. A method for reducing one or more symptoms associated with seasonality in an individual, the method comprising administering to the individual suffering from a seasonality disorder, a composition comprising a therapeutically effective amount of an agent to down regulate the activity of an orexin selected from the group consisting of orexin A or orexin B, at one or more orexin receptors.

11. A method for reducing one or more seasonal symptoms associated with bipolar disorder in an individual, the method comprising administering to the individual suffering from seasonal symptoms associated with bipolar disorder, a composition comprising a therapeutically effective amount of an agent to down regulate the activity of an orexin selected from the group consisting of orexin A or orexin B, at one or more orexin receptors.

12. The method of claim 11, wherein the seasonal symptoms associated with bipolar disorder are selected from the group consisting of manic episodes, hypomanic episodes, mixed manic and hypomanic episodes, and depression.

13. The method of claim 11 wherein orexin receptor activity is down regulated at an orexin 1 receptor.

14. The method of claim 11 wherein orexin receptor activity is down regulated at an orexin 2 receptor.

15. The method of claim 11 wherein the orexin receptor activity is down regulated at both orexin 1 and orexin 2 receptors.

16. The method of claim 11 wherein the agent comprises an orexin receptor antagonist selected from the group consisting of an orexin receptor antagonist selective for orexin 1 receptors, an orexin receptor antagonist selective for orexin 2 receptors, and an orexin receptor antagonist having affinity at orexin 1 receptors and orexin 2 receptors.

17. A method for reducing one or more seasonal symptoms associated with premenstrual syndrome in an individual, the method comprising administering to the individual suffering from seasonal symptoms associated with premenstrual syndrome, a composition comprising a therapeutically effective amount of an agent to down regulate the activity of an orexin selected from the group consisting of orexin A or orexin B, at one or more orexin receptors.

18. The method of claim 17, wherein the seasonal symptoms associated with premenstrual syndrome are selected from the group consisting of irritability, mood swings, crying episodes, and depression.

19. The method of claim 17 wherein the agent comprises an orexin receptor antagonist selected from the group consisting of an orexin receptor antagonist selective for orexin 1 receptors, an orexin receptor antagonist selective for orexin 2 receptors, and an orexin receptor antagonist having affinity at orexin 1 receptors and orexin 2 receptors.

20. The method of claim 17 wherein the agent comprises an orexin receptor antagonist with affinity at both orexin 1 and orexin 2 receptors.

21. A method of determining whether an individual is responsive to a treatment for seasonal affective disorder, the method comprising determining the levels of orexin in a biological sample from the individual obtained once in the spring or summer and once during the fall or winter prior to said treatment, and determining a difference in level of orexin; administering said treatment for period of time to down regulate orexin receptor activity; determining the levels of orexin in a biological sample from the individual during or after said treatment period, comparing the levels of orexin present in the sample obtained after initiating treatment to a baseline level of orexin present in the sample obtained prior to treatment during spring or summer, and determining that the individual is responsive to treatment if orexin levels are reduced during or after said treatment period.

22. The method of claim 20 wherein the biological sample is cerebro-spinal fluid.
